SEQUENCE LISTING (109 sequences)

SEQ ID NO: 1 - protein, 9 aa, Homo Sapiens
SLLFLLFSL

SEQ ID NO: 2 - protein, 9 aa, Homo Sapiens
VLPLTVAEV

SEQ ID NO: 3 - protein, 12 aa, Homo Sapiens
CAASGNTDKLIF

SEQ ID NO: 4 - protein, 13 aa, Homo Sapiens
CAFYMDSNYQLIW

SEQ ID NO: 5 - protein, 13 aa, Homo Sapiens
CAVIPNNNARLMF

SEQ ID NO: 6 - protein, 13 aa, Homo Sapiens
CASRPGWSYEQYF

SEQ ID NO: 7 - protein, 12 aa, Homo Sapiens
CASSEWTAEQYF

SEQ ID NO: 8 - protein, 11 aa, Homo Sapiens
CASGQGTEAFF

SEQ ID NO: 9 - protein, 13 aa, Homo Sapiens
CAYLGTGTYKYIF

SEQ ID NO: 10 - protein, 16 aa, Homo Sapiens
CAGGMESGGGADGLTF

SEQ ID NO: 11 - protein, 12 aa, Homo Sapiens
CALDTGFQKLVF

SEQ ID NO: 12 - protein, 12 aa, Homo Sapiens
CASSSGGLGYTF

SEQ ID NO: 13 - protein, 16 aa, Homo Sapiens
CASTSTGGLKNTEAFF

SEQ ID NO: 14 - protein, 15 aa, Homo Sapiens
CASSSLGDRNTEAFF

SEQ ID NO: 15 - DNA, 36 bp, Artificial Sequence
  Synthetic
tgtgccgccagcggcaacaacgacaagctgatcttt

SEQ ID NO: 16 - DNA, 39 bp, Artificial Sequence
  Synthetic
tgcgccagcagacccggctggtcctacgagcagtatttc

SEQ ID NO: 17 - DNA, 399 bp, Artificial Sequence
  Synthetic
atggatacttggctcgtgtgctgggccatcttcagcctgctgaaggccggactgaccgagcccgaagtgacccagacacctagccaccaagtgacacagatgggccaggaagtgatcctgcgctgcgtgcccatcagcaaccacctgtacttctactggtacagacagatcctggggcagaaagtggaattcctggtgtccttctacaacaacgagatcagcgagaagtccgagatcttcgacgaccagttcagcgtggaacggcccgacggcagcaacttcaccctgaagatcagaagcaccaagctggaagatagcgccatgtacttttgcgccagcagacccggctggtcctacgagcagtatttcggccctggcacccggctgaccgtgaccgag

SEQ ID NO: 18 - DNA, 534 bp, Artificial Sequence
  Synthetic
gatctgaagaacgtgttccccccagaggtggccgtgttcgagccttctgaggccgagatctcccacacccagaaagccaccctcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggatagagccaagcccgtgactcagatcgtgtccgccgaagcttggggcagagccgattgcggctttaccagcgagagctaccagcagggcgtgctgagcgccaccatcctgtacgagatcctgctgggcaaggccaccctgtacgccgtgctggtgtctgccctggtgctgatggccatggtcaagcggaaggacagcagaggc

SEQ ID NO: 19 - DNA, 66 bp, Artificial Sequence
  Synthetic
ggttccggagccacgaacttctctctgttaaagcaagcaggagacgtggaagaaaaccccggtccc

SEQ ID NO: 20 - DNA, 414 bp, Artificial Sequence
  Synthetic
atggctatgctgctgggcgcctctgtgctgatcctgtggctgcagcccgactgggtcaacagccagcagaagaatgacgaccagcaagtgaagcagaactcccccagcctgagcgtgcaggaaggcagaatcagcatcctgaactgcgactacaccaactctatgttcgactacttcctgtggtacaagaagtaccccgccgagggccccaccttcctgatcagcatcagcagcatcaaggacaagaacgaggacggccggttcaccgtgtttctgaacaagagcgccaagcacctgagcctgcacatcgtgcctagccagcctggcgattccgccgtgtatttctgtgccgccagcggcaacaccgacaagctgatctttggcaccggcaccagactgcaggtgttccccaac

SEQ ID NO: 21 - DNA, 423 bp, Artificial Sequence
  Synthetic
atccagaaccccgaccctgccgtgtaccagctgagagacagcaagagcagcgacaagagcgtgtgcctgttcaccgacttcgacagccagaccaacgtgtcccagagcaaggacagcgacgtgtacatcaccgataagaccgtgctggacatgcggagcatggacttcaagagcaacagcgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgtccgtgatcggcttcagaatcctgctgctgaaagtggccggcttcaatctgctgatgaccctgcggctgtggtccagctga

SEQ ID NO: 22 - DNA, 1836 bp, Artificial Sequence
  Synthetic
atggatacttggctcgtgtgctgggccatcttcagcctgctgaaggccggactgaccgagcccgaagtgacccagacacctagccaccaagtgacacagatgggccaggaagtgatcctgcgctgcgtgcccatcagcaaccacctgtacttctactggtacagacagatcctggggcagaaagtggaattcctggtgtccttctacaacaacgagatcagcgagaagtccgagatcttcgacgaccagttcagcgtggaacggcccgacggcagcaacttcaccctgaagatcagaagcaccaagctggaagatagcgccatgtacttttgcgccagcagacccggctggtcctacgagcagtatttcggccctggcacccggctgaccgtgaccgaggatctgaagaacgtgttccccccagaggtggccgtgttcgagccttctgaggccgagatctcccacacccagaaagccaccctcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggatagagccaagcccgtgactcagatcgtgtccgccgaagcttggggcagagccgattgcggctttaccagcgagagctaccagcagggcgtgctgagcgccaccatcctgtacgagatcctgctgggcaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatggctatgctgctgggcgcctctgtgctgatcctgtggctgcagcccgactgggtcaacagccagcagaagaatgacgaccagcaagtgaagcagaactcccccagcctgagcgtgcaggaaggcagaatcagcatcctgaactgcgactacaccaactctatgttcgactacttcctgtggtacaagaagtaccccgccgagggccccaccttcctgatcagcatcagcagcatcaaggacaagaacgaggacggccggttcaccgtgtttctgaacaagagcgccaagcacctgagcctgcacatcgtgcctagccagcctggcgattccgccgtgtatttctgtgccgccagcggcaacaccgacaagctgatctttggcaccggcaccagactgcaggtgttccccaacatccagaaccccgaccctgccgtgtaccagctgagagacagcaagagcagcgacaagagcgtgtgcctgttcaccgacttcgacagccagaccaacgtgtcccagagcaaggacagcgacgtgtacatcaccgataagaccgtgctggacatgcggagcatggacttcaagagcaacagcgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgtccgtgatcggcttcagaatcctgctgctgaaagtggccggcttcaatctgctgatgaccctgcggctgtggtccagctga

SEQ ID NO: 23 - protein, 138 aa, Artificial Sequence
  Synthetic
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGNTDKLIFGTGTRLQVFPN

SEQ ID NO: 24 - protein, 140 aa, Artificial Sequence
  Synthetic
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 25 - protein, 278 aa, Artificial Sequence
  Synthetic
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 133 aa, Artificial Sequence
  Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRPGWSYEQYFGPGTRLTVTE

SEQ ID NO: 27 - protein, 178 aa, Artificial Sequence
  Synthetic
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 28 - protein, 311 aa, Artificial Sequence
  Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRPGWSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 29 - protein, 611 aa, Artificial Sequence
  Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRPGWSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - DNA, 39 bp, Artificial Sequence
  Synthetic
tgcgccttctacatggacagcaactaccagctgatctgg

SEQ ID NO: 31 - DNA, 36 bp, Artificial Sequence
  Synthetic
tgtgccagcagcgagtggaccgccgagcagtatttt

SEQ ID NO: 32 - DNA, 1839 bp, Artificial Sequence
  Synthetic
tggcgcgccaccatgtctatcggcctgctgtgctgcgtggccttcagtctgctgtgggccagccctgtgaatgccggcgtgacccagacccccaagttccaggtgctgaaaaccggccagagcatgaccctgcagtgcgcccaggacatgaaccacaacagcatgtactggtacagacaggaccccggcatgggcctgcggctgatctactactctgccagcgagggcaccaccgacaagggcgaagtgcccaacggctacaacgtgtcccggctgaacaagagagagttcagcctgagactggaaagcgccgctcccagccagaccagcgtgtacttttgtgccagcagcgagtggaccgccgagcagtattttggccctggcaccagactgaccgtgaccgaggacctgaagaacgtgttccccccagaggtggccgtgttcgagccttctgaggccgagatcagccacacccagaaagccaccctcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgtccagcaggctgagagtgtccgccaccttctggcagaacccccggaaccacttcagatgccaggtgcagttctacggcctgagcgagaacgatgagtggacccaggacagagccaagcccgtgacacagatcgtgtctgccgaagcctggggcagagccgattgcggctttaccagcgagagctaccagcagggcgtgctgagcgccacaatcctgtacgagatcctgctgggcaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatggaaaagaaccccctggccgctcccctgctgatcctgtggtttcacctggactgcgtgtcctccatcctgaacgtggaacagagcccccagagcctgcatgtgcaggaaggcgacagcaccaacttcacctgtagcttccccagcagcaacttctacgccctgcactggtatagatgggagacagccaagagccccgaggccctgttcgtgatgaccctgaacggcgacgagaagaagaagggccggatcagcgccaccctgaacaccaaagagggctacagctacctgtacatcaagggcagccagcccgaggacagcgccacatacctgtgcgccttctacatggacagcaactaccagctgatctggggagccggcaccaagctgatcatcaagcccgacatccagaaccccgaccccgccgtgtatcagctgagggacagcaagagcagcgacaagagcgtgtgcctgttcaccgacttcgactcccagaccaatgtgtcccagagcaaggacagcgacgtgtacattaccgacaagaccgtgctggacatgcggagcatggacttcaagagcaacagcgccgtggcctggtccaacaagagcgatttcgcctgcgccaacgccttcaacaactccattatccctgaggacacattcttcccaagccccgagagcagctgcgacgtgaagctggtggaaaagagcttcgagacagacaccaacctgaacttccagaacctgagcgtgatcggcttcagaatcctgctgctgaaggtggccggcttcaacctgctgatgacactgcggctgtggtccagctgagtcgac

SEQ ID NO: 33 - protein, 135 aa, Artificial Sequence
  Synthetic
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFYMDSNYQLIWGAGTKLIIKPD

SEQ ID NO: 34 - protein, 140 aa, Artificial Sequence; identical to SEQ ID NO: 24
  Synthetic

SEQ ID NO: 35 - protein, 275 aa, Artificial Sequence
  Synthetic
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFYMDSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 131 aa, Artificial Sequence
  Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEWTAEQYFGPGTRLTVTE

SEQ ID NO: 37 - protein, 178 aa, Artificial Sequence; identical to SEQ ID NO: 27
  Synthetic

SEQ ID NO: 38 - protein, 309 aa, Artificial Sequence
  Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEWTAEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 39 - protein, 606 aa, Artificial Sequence
  Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEWTAEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFYMDSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - DNA, 39 bp, Artificial Sequence
  Synthetic
tgcgccgtgatccccaacaacaacgcccggctgatgttt

SEQ ID NO: 41 - DNA, 33 bp, Artificial Sequence
  Synthetic
tgtgcctctggccagggaaccgaggcattcttt

SEQ ID NO: 42 - DNA, 1829 bp, Artificial Sequence
  Synthetic
tggcgcgccaccatgggatgcagactgctgtgttgcgccgtgctgtgtctgctgggagccgtgcctatggaaaccggcgtgacccagacccccagacacctcgtgatgggcatgaccaacaagaaaagcctgaagtgcgagcagcacctgggccacaacgccatgtactggtacaagcagagcgccaagaaacccctggaactgatgttcgtgtacaacttcaaagagcagaccgagaacaacagcgtgcccagcagattcagccccgagtgccccaatagcagccacctgtttctgcatctgcacaccctgcagcccgaggacagcgccctgtatctgtgtgcctctggccagggaaccgaggcattctttgggcagggcaccagactgaccgtggtggaagatctgaacaaggtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttaccagcgtgtcctatcagcagggcgtgctgagcgccacaatcctgtacgagatcctgctgggaaaggccaccctgtatgcagtgctggtgtccgccctggtgctgatggccatggtcaagcggaaggacttcggttccggagccacgaacttctctctgttaaagcaagcaggagacgtggaagaaaaccccggtcccgtcccatgctcctgctgctggtgcctgccttccaagtgatcttcaccctgggcggcaccagagcccagtctgtgacccagctggatagccaggtgcccgtgtttgaagaggcccctgtggaactgcggtgcaactacagcagctccgtgtccgtgtacctgttttggtacgtgcagtaccccaaccagggcctgcagctgctgctgaagtacctgagcggcagcaccctcgtgaagggaatcaacggcttcgaggccgaattcaacaagagccagaccagcttccacctgagaaagcccagcgtgcacatcagcgataccgccgagtacttctgcgccgtgatccccaacaacaacgcccggctgatgtttggcgacggcacacagctggtcgtgaagcccaacatccagaaccccgaccccgccgtgtaccagctgagagacagcaagagcagcgacaagagcgtgtgtctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagtccgatttcgcctgcgccaacgccttcaacaacagcattatccctgaggacacattcttcccaagccccgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtccagctgagtcgac

SEQ ID NO: 43 - protein, 133 aa, Artificial Sequence
  Synthetic
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVIPNNNARLMFGDGTQLVVKPN

SEQ ID NO: 44 - protein, 140 aa, Artificial Sequence; identical to SEQ ID NO: 24
  Synthetic

SEQ ID NO: 45 - protein, 273 aa, Artificial Sequence
  Synthetic
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVIPNNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 130 aa, Artificial Sequence
  Synthetic
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASGQGTEAFFGQGTRLTVVE

SEQ ID NO: 47 - protein, 176 aa, Artificial Sequence
  Synthetic
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 48 - protein, 306 aa, Artificial Sequence
  Synthetic
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASGQGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 49 - protein, 601 aa, Artificial Sequence
  Synthetic
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASGQGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVIPNNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - DNA, 39 bp, Artificial Sequence
  Synthetic
tgtgcctacctgggcaccggcacctacaagtacatcttc

SEQ ID NO: 51 - DNA, 36 bp, Artificial Sequence
  Synthetic
tgcgcctctagctctggcggcctgggatacacattt

SEQ ID NO: 52 - DNA, 393 bp, Artificial Sequence
  Synthetic
atgggacctcagctgctgggatacgtggtgctgtgtctgctgggagccggacctctggaagcccaagtgacccagaaccccagatacctgatcaccgtgaccggcaagaaactgaccgtgacctgcagccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgcctctagctctggcggcctgggatacacatttggcagcggcaccaggctgaccgtggtggaa

SEQ ID NO: 53 - DNA, 528 bp, Artificial Sequence
  Synthetic
gatctgaacaaggtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttaccagcgtgtcctatcagcagggcgtgctgagcgccacaatcctgtacgagatcctgctgggcaaggccaccctgtacgccgtgctggtgtccgctctggtgctgatggccatggtcaagcggaaggacttc

SEQ ID NO: 54 - DNA, 66 bp, Artificial Sequence; identical to SEQ ID NO: 19
  Synthetic

SEQ ID NO: 55 - DNA, 405 bp, Artificial Sequence
  Synthetic
atggcctgccccggatttctgtgggccctcgtgatcagcacctgtctggaattcagcatggcccagaccgtgactcagtcccagcccgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagtccttcagcctgaagatcagcgacagccagctgggcgacgccgccatgtacttttgtgcctacctgggcaccggcacctacaagtacatcttcggcacaggcacccggctgaaggtgctggccaac

SEQ ID NO: 56 - DNA, 422 bp, Artificial Sequence
  Synthetic
atccagaaccctgaccccgccgtgtatcagctgcgggacagcaagagcagcgacaagagcgtgtgtctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagccccgagagcagctgcgacgtgaagctggtggaaaagagcttcgagacagacaccaacctgaatttccagaacctgagcgtgatcggcttcagaatcctgctgctgaaagtggccggcttcaacctgctgatgaccctgcggctgtggtccagctg

SEQ ID NO: 57 - DNA, 1814 bp, Artificial Sequence
  Synthetic
atgggacctcagctgctgggatacgtggtgctgtgtctgctgggagccggacctctggaagcccaagtgacccagaaccccagatacctgatcaccgtgaccggcaagaaactgaccgtgacctgcagccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgcctctagctctggcggcctgggatacacatttggcagcggcaccaggctgaccgtggtggaagatctgaacaaggtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttaccagcgtgtcctatcagcagggcgtgctgagcgccacaatcctgtacgagatcctgctgggcaaggccaccctgtacgccgtgctggtgtccgctctggtgctgatggccatggtcaagcggaaggacttcggttccggagccacgaacttctctctgttaaagcaagcaggagacgtggaagaaaaccccggtcccatggcctgccccggatttctgtgggccctcgtgatcagcacctgtctggaattcagcatggcccagaccgtgactcagtcccagcccgagatgagcgtgcaggaagccgagacagtgaccctgagctgcacctacgacaccagcgagagcgactactacctgttctggtacaagcagccccccagccggcagatgatcctcgtgattagacaggaagcctataagcagcagaacgccaccgagaacagattcagcgtgaacttccagaaggccgccaagtccttcagcctgaagatcagcgacagccagctgggcgacgccgccatgtacttttgtgcctacctgggcaccggcacctacaagtacatcttcggcacaggcacccggctgaaggtgctggccaacatccagaaccccgaccctgccgtgtaccagctgagagacagcaagagcagcgacaagagcgtgtgcctgttcaccgacttcgacagccagaccaacgtgtcccagagcaaggacagcgacgtgtacatcaccgataagaccgtgctggacatgcggagcatggacttcaagagcaacagcgccgtggcctggtccaacaagtccgatttcgcctgcgccaacgccttcaacaacagcattatccccgaggacacattcttcccaagccccgagagcagctgcgacgtgaagctggtggaaaagagcttcgagacagacaccaacctgaacttccagaacctgtccgtgatcggcttcagaatcctgctgctgaaagtggccggcttcaatctgctgatgaccctgcggctgtggtccagctg

SEQ ID NO: 58 - protein, 135 aa, Artificial Sequence
  Synthetic
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLGTGTYKYIFGTGTRLKVLAN

SEQ ID NO: 59 - protein, 140 aa, Artificial Sequence; identical to SEQ ID NO: 24
  Synthetic

SEQ ID NO: 60 - protein, 275 aa, Artificial Sequence
  Synthetic
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLGTGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 61 - protein, 131 aa, Artificial Sequence
  Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSGGLGYTFGSGTRLTVVE

SEQ ID NO: 62 - protein, 176 aa, Artificial Sequence; identical to SEQ ID NO: 47
  Synthetic

SEQ ID NO: 63 - protein, 307 aa, Artificial Sequence
  Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSGGLGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 64 - protein, 604 aa, Artificial Sequence
  Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSGGLGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLGTGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 65 - DNA, 48 bp, Artificial Sequence
  Synthetic
tgcgctggcggaatggaatctggcggcggagccgatggcctgaccttt

SEQ ID NO: 66 - DNA, 48 bp, Artificial Sequence
  Synthetic
tgtgccagcacaagcacaggcggcctgaagaacaccgaggcattcttt

SEQ ID NO: 67 - DNA, 1848 bp, Artificial Sequence
  Synthetic
atggcgcgccaccatgggaacaagcctgctgtgttggatggccctgtgtctgctgggagccgaccatgccgatacaggcgtgtcccagaacccccggcacaagatcaccaagcggggccagaacgtgaccttcagatgcgaccccatcagcgagcacaaccggctgtactggtacagacagaccctgggccagggccccgagttcctgacctacttccagaacgaggcccagctggaaaagagccggctgctgagcgacagattcagcgccgaaagacccaagggcagcttcagcaccctggaaatccagcggaccgagcagggcgacagcgccatgtatctgtgtgccagcacaagcacaggcggcctgaagaacaccgaggcattctttgggcagggcacccggctgaccgtggtggaagatctgaacaaggtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcaggtgccaggtgcagttctacggcctgagcgagaacgacgagtggacccaggacagagccaagcccgtgacccagatcgtgtctgccgaagcctggggcagagccgattgcggctttaccagcgtgtcctatcagcagggcgtgctgagcgccacaatcctgtacgagatcctgctgggcaaggccaccctgtacgccgtgctggtgtctgccctggtgctgatggccatggtcaagcggaaggacttcggttccggagccacgaacttctctctgttaaagcaagcaggagacgtggaagaaaaccccggtcccgtcccatggtgctgaagttctccgtgtccatcctgtggatccagctggcctgggtgtccacccagctgctggaacagtcccctcagttcctgagcatccaggaaggcgagaacctgaccgtgtactgcaacagcagcagcgtgttcagcagcctgcagtggtacaggcaggaaccaggcgagggaccagtgctgctcgtgactgtcgtgacaggcggcgaagtgaagaagctgaagcggctgaccttccagttcggcgacgccagaaaggacagctccctgcacattacagccgcccagacaggcgacaccggcctgtacctgtgcgctggcggaatggaatctggcggcggagccgatggcctgacctttggcaagggcacacacctgatcatccagccctacatccagaatcccgaccccgccgtgtaccagctgagagacagcaagagcagcgacaagagcgtgtgtctgttcaccgacttcgacagccagaccaatgtgtcccagtccaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagccccgagagcagctgcgacgtgaagctggtggaaaagtccttcgagacagacaccaacctgaatttccagaatctgagcgtgatcggcttccgcatcctgctgctgaaggtggccggcttcaacctgctgatgaccctgagactgtggtcctcctgagtcgac

SEQ ID NO: 68 - protein, 133 aa, Artificial Sequence
  Synthetic
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQTGDTGLYLCAGGMESGGGADGLTFGKGTHLIIQPY

SEQ ID NO: 69 - protein, 140 aa, Artificial Sequence; identical to SEQ ID NO: 24
  Synthetic

SEQ ID NO: 70 - protein, 273 aa, Artificial Sequence
  Synthetic
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQTGDTGLYLCAGGMESGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 71 - protein, 136 aa, Artificial Sequence
  Synthetic
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASTSTGGLKNTEAFFGQGTRLTVVE

SEQ ID NO: 72 - protein, 176 aa, Artificial Sequence; identical to SEQ ID NO: 47
  Synthetic

SEQ ID NO: 73 - protein, 312 aa, Artificial Sequence
  Synthetic
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASTSTGGLKNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 74 - protein, 607 aa, Artificial Sequence
  Synthetic
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASTSTGGLKNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQTGDTGLYLCAGGMESGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 75 - DNA, 36 bp, Artificial Sequence
  Synthetic
tgcgccctggataccggctttcagaaactggtgttc

SEQ ID NO: 76 - DNA, 45 bp, Artificial Sequence
  Synthetic
tgtgccagcagcagcctgggcgaccggaacaccgaggcattcttt

SEQ ID NO: 77 - DNA, 1836 bp, Artificial Sequence
  Synthetic
atggcgcgccaccatgggacctcagctgctgggatacgtggtgctgtgtctgctgggagccggacctctggaagcccaagtgacccagaaccccagatacctgatcaccgtgaccggcaagaaactgaccgtgacctgcagccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgtgccagcagcagcctgggcgaccggaacaccgaggcattctttgggcagggcacccggctgaccgtggtggaagatctgaacaaggtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttaccagcgtgtcctatcagcagggcgtgctgagcgccacaatcctgtacgagatcctgctgggcaaggccaccctgtacgccgtgctggtgtcagccctggtgctgatggccatggtcaagcggaaggacttcggttccggagccacgaacttctctctgttaaagcaagcaggagacgtggaagaaaaccccggtcccgtcccatgaactacagccctggcctggtgtccctgattctcctgctgctggggcggaccagaggcaactccgtgactcagatggaaggccccgtgaccctgagcgaagaggccttcctgaccatcaattgcacctacaccgccacaggctaccccagcctgttttggtacgtgcagtaccccggcgagggactgcagctgctgctgaaggccaccaaggccgacgataagggcagcaacaagggcttcgaggccacctacagaaaagagacaaccagcttccacctggaaaagggcagcgtgcaggtgtccgacagcgccgtgtatttctgcgccctggataccggctttcagaaactggtgttcggcaccggcaccagactgctggtgtcccccaacatccagaaccccgaccctgccgtgtatcagctgcgggacagcaagagcagcgacaagagcgtgtgtctgttcaccgacttcgacagccagaccaacgtgtcccagagcaaggactccgacgtgtacatcaccgacaagaccgtgctggacatgcggagcatggacttcaagagcaactccgccgtggcctggtccaacaagagcgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttccggatcctgctgctgaaagtggccggcttcaacctgctgatgaccctgcggctgtggtccagctgagtcgac

SEQ ID NO: 78 - protein, 131 aa, Artificial Sequence
  Synthetic
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALDTGFQKLVFGTGTRLLVSPN

SEQ ID NO: 79 - protein, 140 aa, Artificial Sequence; identical to SEQ ID NO: 24
  Synthetic

SEQ ID NO: 80 - protein, 271 aa, Artificial Sequence
  Synthetic
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALDTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 81 - protein, 134 aa, Artificial Sequence
  Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSLGDRNTEAFFGQGTRLTVVE

SEQ ID NO: 82 - protein, 176 aa, Artificial Sequence; identical to SEQ ID NO: 47
  Synthetic

SEQ ID NO: 83 - protein, 310 aa, Artificial Sequence
  Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSLGDRNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 84 - protein, 603 aa, Artificial Sequence
  Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSLGDRNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALDTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 85 - protein, 630 aa, Homo Sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 86 - protein, 567 aa, Homo Sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 87 - protein, 503 aa, Homo Sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 88 - protein, 12 aa, Artificial Sequence
  Synthetic
DQQVKQNSPSLS

SEQ ID NO: 89 - protein, 10 aa, Artificial Sequence
  Synthetic
MFDYFLWYKK

SEQ ID NO: 90 - protein, 12 aa, Artificial Sequence
  Synthetic
PSHQVTQMGQEV

SEQ ID NO: 91 - protein, 10 aa, Artificial Sequence
  Synthetic
QILGQKVEFL

SEQ ID NO: 92 - protein, 12 aa, Artificial Sequence
  Synthetic
EQSPQSLHVQEG

SEQ ID NO: 93 - protein, 10 aa, Artificial Sequence
  Synthetic
HWYRWETAKS

SEQ ID NO: 94 - protein, 12 aa, Artificial Sequence
  Synthetic
PKFQVLKTGQSM

SEQ ID NO: 95 - protein, 11 aa, Artificial Sequence
  Synthetic
QDPGMGLRLIY

SEQ ID NO: 96 - protein, 12 aa, Artificial Sequence
  Synthetic
DSQVPVFEEAPV

SEQ ID NO: 97 - protein, 9 aa, Artificial Sequence
  Synthetic
PNQGLQLLL

SEQ ID NO: 98 - protein, 12 aa, Artificial Sequence
  Synthetic
LVCLATGFFPDH

SEQ ID NO: 99 - protein, 10 aa, Artificial Sequence
  Synthetic
TDPQPLKEQP

SEQ ID NO: 100 - protein, 12 aa, Artificial Sequence
  Synthetic
SQPEMSVQEAET

SEQ ID NO: 101 - protein, 10 aa, Artificial Sequence
  Synthetic
KQPPSRQMIL

SEQ ID NO: 102 - protein, 12 aa, Artificial Sequence
  Synthetic
TLVCLATGFFPD

SEQ ID NO: 103 - protein, 10 aa, Artificial Sequence
  Synthetic
STDPQPLKEQ

SEQ ID NO: 104 - protein, 12 aa, Artificial Sequence
  Synthetic
QFLSIQEGENLT

SEQ ID NO: 105 - protein, 10 aa, Artificial Sequence
  Synthetic
GEGPVLLVTV

SEQ ID NO: 106 - protein, 12 aa, Artificial Sequence
  Synthetic
EGPVTLSEEAFL

SEQ ID NO: 107 - protein, 10 aa, Artificial Sequence
  Synthetic
YPGEGLQLLL

SEQ ID NO: 108 - protein, 12 aa, Artificial Sequence
  Synthetic
TLVCLATGFFPD

SEQ ID NO: 109 - protein, 9 aa, Artificial Sequence
  Synthetic
PALNDSRYC

(19) United States
(12) Patent Application Publication
(10) Pub. No.: US 2024/0209058 A1
(43) Pub. Date: Jun. 27, 2024

(54) Mesothelin-specific T cell Receptors and Methods of Using Same

(71) Applicants: REGENTS OF THE UNIVERSITY OF MINNESOTA, Minneapolis, MN (US); FRED HUTCHINSON CANCER CENTER, Seattle, WA (US)

(72) Inventors: Ingunn Stromnes, Minneapolis, MN (US); Philip D. Greenberg, Seattle, WA (US); Thomas M. Schmitt, Seattle, WA (US)

(21) Appl. No.: 18/288,420

(22) PCT Filed: Apr. 29, 2022
(86) PCT No.: PCT/US22/27065

§ 371 Date: Oct. 26, 2023

Related U.S. Application Data
(60) Provisional application No. 63/182,227, filed on Apr. 30, 2021.

(51) Int. Cl. C07K 14/725; A61K 38/00; A61K 39/00; A61P 35/00

(52) U.S. Cl. CPC C07K 14/7051; A61K 39/4632; A61K 39/464468; A61P 35/00; A61K 38/00

(57) ABSTRACT

Mesothelin-specific binding proteins including mesothelin-specific T cell receptor (TCR) that specifically binds to mesothelin including. for example. in a complex with an MHC. Methods of making and using the mesothelin-specific binding proteins are also described, including the use of the mesothelin-specific binding protein in adoptive cell therapy.

### CROSS-REFERENCE TO RELATED APPLICATIONS AND INCORPORATION OF MATERIALS SUBMITTED ELECTRONICALLY

This application claims the benefit of priority to U.S. Provisional Patent Application No. 63/182,227, filed Apr. 30, 2021, which is hereby incorporated by reference in its entirety. Incorporated by reference in its entirety is a computer-readable nucleotide/amino acid sequence listing submitted concurrently herewith and identified as follows: 140,364 byte ASCII (Text) file named “56947_Seqlisting.txt”; created on Apr. 29, 2022.

### GOVERNMENT FUNDING

This invention was made with government support under grant number CA015704, CA018029, and CA033084 awarded by the National Institutes of Health. The government has certain rights in the invention.

### BACKGROUND

T lymphocytes can specifically recognize and kill cancer cells by expression of a tumor-antigen specific T cell receptor (TCR). Most TCRs are a heterodimer of a TCRα and TCRβ chain and are generated during T cell development through a process of gene (VJ and VDJ) recombination. TCRs recognize intracellular peptides in the context of human leukocyte antigen (HLA) molecules in humans. HLA molecules are highly polymorphic genes and exhibit the most variability within the peptide binding region (Little et al. Rev Immunogenet 1, 105-123 (1999)). During T cell development, most T cells that express a strongly reactive TCR specific to self-peptide:HLA are deleted or tolerized (Starr et al. Annu Rev Immunol 21, 139-176 (2003)). T cells that express a TCR that fails to sufficiently recognize HLA die by neglect, resulting in a TCR repertoire that is HLA-restricted, biased toward foreign antigen recognition, highly diverse, specific, and unique for each individual. Rare peptides that strongly bind HLA and elicit a T cell response are immunogenic.

Adoptive cell therapy (ACT) involves the ex vivo expansion, often genetic manipulation, and infusion of tumor-reactive T cells into cancer patients. The adoptive transfer of T cells that express a tumor-reactive TCR have shown efficacy for some solid tumors. For most therapies, TCRs of high affinity are selected for targeting cancer with engineered T cells. It is assumed that a higher affinity TCR will provide superior anti-tumor activity by increasing T cell recognition of antigen (for example, peptide:HLA complexes) on the tumor cell surface. However, efforts to enhance the affinity of TCRs to self/tumor antigens have resulted in lethal toxicity.

### SUMMARY

In one aspect, this disclosure describes a mesothelin-specific binding protein, that is, a protein or polypeptide that specifically binds to mesothelin (including a peptide or fragment thereof). In some embodiments, the mesothelin-specific binding protein binds to mesothelin (or a peptide or fragment thereof) complexed with an MHC. A mesothelin-specific binding protein may include a mesothelin-specific T cell receptor (TCR). The mesothelin-specific binding protein may be used in adoptive cell therapy (ACT). In some embodiments, the mesothelin-specific binding protein is sufficiently avid to mediate lysing of a tumor in an antigen-specific manner but does not have sufficiently high affinity to result in off-tumor toxicity.

In a further aspect, this disclosure describes methods of delivering a mesothelin-specific binding protein to a target cell to produce a cell that overexpresses the mesothelin-specific binding protein. The disclosure further provides a method comprising delivering a construct that encodes a mesothelin-specific binding protein of a target cell to produce a cell that overexpresses the mesothelin-specific binding protein. In some embodiments, the target call may be a T cell or another cell type, such as a cell type that can express a T cell receptor. In some embodiments, the mesothelin-specific binding protein may preferably include a T cell receptor.

In another aspect, this disclosure also describes cells that overexpress the mesothelin-specific binding proteins described herein and methods of using those cells. In some embodiments, a cell that over expresses a mesothelin-specific binding protein may also overexpress a molecule that improves the immune response to mesothelin or to a tumor expressing mesothelin. Such molecules include, for example, a molecule that interferes with inhibitory receptor expression; a molecule that interferes with suppressive cytokine signaling; a molecule that renders T cells resistant to a program of T cell exhaustion and/or promotes resident memory; a chimeric costimulatory receptor; an anti-tumor factor; or a combination thereof. In various aspects, the cell is modified to reduce expression of, or altering the signally via, a transforming growth factor beta (TGFβ) receptor, such as TGFβR2 or TGFβR1.

The disclosure further provides a method of treating a mesothelin-positive malignancy in a subject in need thereof, the method comprising administering to the subject a composition comprising cells that overexpress the mesothelin-specific binding proteins described herein. Optionally, method further comprises administering a mesothelin peptide or a construct encoding a mesothelin peptide, a CD40 agonist, an adjuvant, and/or a cytokine to the subject.

As used herein “isolated” means that the material is removed from its original environment (for example, the natural environment if it is naturally occurring). For example, a naturally occurring nucleic acid or polypeptide present in a living animal is not isolated, but the same nucleic acid or polypeptide, separated from some or all of the co-existing materials in the natural system, is isolated.

As used herein, “sequence identity” between two polypeptides refers to the percentage of amino acid residues in one polypeptide sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values may be generated using the NCBI BLAST2.0 software (Altschul et al. Nucleic Acids Res 25, 3389-3402 (1997)), with the parameters set to default values.

A “conservative substitution” is recognized in the art as a substitution of one amino acid for another amino acid that has similar properties. Exemplary conservative substitutions are well known in the art (see, e.g., International Patent Publication No. WO 1997/09433 at page 10).

As used herein “treating” or “treatment” is not intended to be an absolute term. Treatment may lead to an improved prognosis or a reduction in the frequency or severity of symptoms. A “therapeutically effective” concentration or amount as used herein is an amount that provides some improvement or benefit to the subject. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In this respect, the methods described herein provide any amount or any level of treatment.

In the context of treating cancer or a malignancy, for instance, the method of the present disclosure may reduce tumor size or mediate tumor cell death, or encompass slowing the progression of the disease (i.e., slowing the growth of a tumor). Treatment for cancer (e.g., a tumor) may be determined by any of a number of ways. Any improvement in the subject's wellbeing is contemplated (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about an 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth or appearance of new lesions; (6) decrease in tumor size or burden; (7) absence of clinically detectable disease, (8) decrease in levels of cancer markers; (9) an increased patient survival rate; and/or (10) some relief from one or more symptoms associated with the disease or condition (e.g., pain). For example, the efficacy of treatment may be determined by detecting a change in tumor mass and/or volume after treatment. The size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). Alternatively, tumor response or cancer response may be characterized in a qualitative fashion like “pathological complete response” (pCR), “clinical complete remission” (cCR), “clinical partial remission” (cPR), “clinical stable disease” (cSD), “clinical progressive disease” (cPD), or other qualitative criteria. In various aspects, the methods of the disclosure further comprise monitoring treatment in the subject.

The term “preventing,” as used herein, is not intended as an absolute term. Instead, prevention refers to delay of onset, reduced frequency of symptoms, or reduced severity of symptoms associated with a disorder. Prevention therefore refers to a broad range of prophylactic measures that will be understood by those in the art, including “inhibition.” In some circumstances, the frequency and severity of symptoms is reduced to non-pathological levels. In some circumstances, the symptoms of an individual receiving the compositions of the disclosure are only 90%, 80%, 70%, 60%, 50%, 40%, 30%, 20%, 10%, 5%, or 1% as frequent or severe as symptoms experienced by an untreated individual with the disorder. The presently disclosed methods may inhibit the spread of the spread or growth of the tumor to any amount or level.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. The disclosure contemplates embodiments described as “comprising” a feature to include embodiments which “consist of” or “consist essentially of” the feature.

Unless otherwise specified, “a,” “an,” “the,” and “at least one” are used interchangeably and mean one or more than one. As used herein, the term “or” is generally employed in its usual sense including “and/or” unless the content clearly dictates otherwise. The term “and/or” means one or all of the listed elements or a combination of any two or more of the listed elements. Also herein, the recitations of numerical ranges by endpoints include all numbers subsumed within that range (for example, 1 to 5 includes 1, 1.5, 2, 2.75, 3, 3.80, 4, 5, etc.). However, the description also contemplates the same ranges in which the lower and/or the higher endpoint is excluded. Herein, “up to” a number (for example, up to 50) includes the number (for example, 50). The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

Reference throughout this specification to “one embodiment,” “an embodiment,” “certain embodiments,” “various aspects,” or “some embodiments,” etc., means that a particular feature, configuration, composition, or characteristic described in connection with the embodiment (or aspect) is included in at least one embodiment (or aspect) of the disclosure. Thus, the appearances of such phrases in various places throughout this specification are not necessarily referring to the same embodiment (or aspect) of the disclosure. Furthermore, the particular features, configurations, compositions, or characteristics may be combined in any suitable manner in one or more embodiments (or aspects). Features of the invention described herein can be re-combined into additional embodiments that also are intended as aspects of the invention, irrespective of whether the combination of features is specified as an aspect or embodiment of the invention.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A shows exemplary results of screening of human T cell lines reactive to mesothelin (MSLN) epitopes (MSLN₂₀₋₂₈ (SLLFLLFSL (SEQ ID NO:1)) or MSLN₅₃₀₋₅₃₈ (VLPLTVAEV (SEQ ID NO:2)) for tetramer binding by flow cytometry.

FIG. 1B shows mean fluorescence intensity (MFI) of tetramer staining for the human T cell lines. Boxes indicate cell lines that bind tetramer particularly well (cell lines 2, 8, 9, 17, 20, and 22).

FIG. 2A-FIG. 2B show screening of human T cell lines reactive to MSLN epitopes for specific lysis of T2 cells pulsed with titrating concentrations of MSLN peptide. FIG. 2A shows exemplary results of incubating independent T cell lines reactive to MSLN₂₀₋₂₈ with T2 cells pulsed with titrating concentrations of MSLN₂₀₋₂₈ peptide; specific lysis was determined by a chromium assay. FIG. 2B shows exemplary results of incubating independent T cell lines reactive to MSLN₅₃₀₋₅₃₈ with T2 cells pulsed with titrating concentrations of MSLN₅₃₀₋₅₃₈ peptide; specific lysis was determined by a chromium release assay.

FIG. 3A-FIG. 3B show exemplary expression of codon-optimized MSLN TCRs (MSLN₂₀₋₂₈ clones 2, 7, and 8 and MSLN₅₃₀₋₅₃₈ clones 4, 5, and 6) in CD8⁺ Jurkat T cells.

FIG. 4A shows exemplary expression of codon-optimized MSLN TCRs (MSLN₂₀₋₂₈ clones 2, 7, and 8, and MSLN₅₃₀₋₅₃₈ clones 4 and 5) in CD8 or CD8⁺ Jurkat T cells. Tetramer staining intensity, which is a surrogate for TCR affinity, was brightest in MSLN₂₀₋₂₈ clone 2 and MSLN₅₃₀₋₅₃₈ clone 4. The MSLN₂₀₋₂₈ TCRs bind tetramer independent of CD8a co-receptor, whereas MSLN₅₃₀₋₅₃₈ TCRs require CD8 co-receptor for tetramer binding, suggesting that the MSLN₂₀₋₂₈ TCRs demonstrate higher affinity for MSLN than the MSLN₅₃₀₋₅₃₈ clones.

FIG. 4B shows tetramer staining of MSLN TCRs transduced into a JURKAT Nur77-reporter cell line. Nur77 is downstream of TCR signaling and, thus, serves as a surrogate for TCR signaling. Clones 2 and 4 stained brightest for their respective tetramers.

FIG. 4C shows exemplary results of independent human tumor cell lines screened for mean fluorescence intensity (MFI) for HLA-A2 and Mesothelin (MSLN) by flow cytometry. Arrows indicate tumor cell lines that were tested as described for FIG. 4D-F. Tumor cell lines express both the target antigen MSLN and HLA-A2 to be recognized by MSLN-specific T cells.

FIG. 4D shows exemplary results of the ability of OVCAR3 ovarian cancer cells to induce TCR signaling in TCR transduced Jurkat cells. (−) no treatment, +rIFNg, tumor cells were pre-incubated with recombinant human IFNγ to increase HLA-A2 (not shown) 24 h prior to incubation with TCR+Jurkats;+peptide represents results observed when the respective MSLN₂₀₋₂₈ or MSLN₅₃₀₋₅₃₈ peptide was pulsed into the tumor cells. Clone 8 and clone 5 outperformed the other TCRs insofar as achieving stronger TCR signaling with and without peptide pulse.

FIG. 4E shows exemplary results of the ability of human HCC1395 colorectal cancer cells to induce TCR signaling in TCR transduced Jurkat cells. (−) no treatment, +rIFNg, tumor cells were pre-incubated with recombinant human IFNγ to increase HLA-A2 24 h prior to incubation with TCR+Jurkats; +peptide represents results observed when MSLN₂₀₋₂₈ or MSLN₅₃₀₋₅₃₈ peptide was pulsed into the tumor cells prior to incubation with T cells. Clone 8 and clone 5 outperformed the other TCRs insofar as achieving stronger TCR signaling with and without peptide pulse.

FIG. 4F shows exemplary results of the ability of human pancreatic cancer Panc01 cells to induce TCR signaling in TCR transduced Jurkat cells. (−) no treatment. +rIFNg, tumor cells were pre-incubated with recombinant human IFNγ to increase HLA-A2 24 h prior to incubation with TCR+Jurkats; +peptide represents results observed when MSLN₂₀₋₂₈ or MSLN₅₃₀₋₅₃₈ peptide was pulsed into the tumor cells prior to incubation with T cells. Clone 8 and clone 5 outperformed the other TCRs insofar as achieving stronger TCR signaling with and without peptide pulse. Pancreatic cancer cells are less immunogenic than the ovarian and colorectal cancer cells (FIG. 4D-4E), which is likely partially due to the lower expression of the target antigen mesothelin and/or HLA-A2 (see FIG. 4C). In summary, the data provided in FIG. 4D-F suggest that TCRs that do not stain brightest for tetramer, and thus are likely lower affinity, surprisingly appear to have enhanced functionality.

FIG. 5 shows exemplary results of the ability of primary human CD8₊ T cells transduced with MSLN TCRs to specifically lyse pancreatic tumor cell line (Panc1). % specific lysis is provided on the y-axis, and effector cell to target cell ratio is provided on the x-axis. Specific tumor cell lysis was measured in vitro by a standard Chromium release assay in triplicate. Results are unexpected as Mesothelin₂₀₋₂₈ clone #2 (diamond) stains brightest for tetramer (FIG. 4), and thus is presumably highest affinity. However, clone 7 (square) and clone 8 (triangle), which exhibit lower tetramer staining than clone 2, and may be lower affinity, mediate more robust Panc01 tumor cell killing. Mesothelin₅₃₀₋₅₃₈ clone 5 is represented by “X”.

FIG. 6A is a line graph illustrating tumor radiance (y-axis) in the pancreas of NSG mice orthotopically implanted with HLA-A2+Mesothelin+Panc01 cell line and either left untreated, or on day 7 post tumor implantation once tumor is established, received 5×10⁶ Mesothelin₂₀₋₂₈ clone 2 TCR+human T cells i.p. Tumor radiance was determined by injection of D-Luciferin and IVIS imaging. Recipients received only a single dose of T cells, without cytokine support or vaccination.

FIG. 6B shows overall survival from mice in FIG. 6A and combined with another experiment. n=4-5 mice per group. Recipients received only a single dose of modified T cells, without cytokine support or vaccination.

FIG. 6C shows mean tumor radiance in the pancreas of NSG mice orthotopically implanted with HLA-A2+Mesothelin+Panc01 cell line and either left untreated, or on day 7 post tumor implantation once tumor is established, received 1×10⁶ Mesothelin₅₃₀₋₅₃₈ clone 4 TCR+human T cells i.p. Tumor radiance was determined by injection of D-Luciferin and IVIS imaging. Recipients received only a single dose of modified T cells, without cytokine support or vaccination.

FIG. 6D shows overall survival from mice in FIG. 6C and combined with another experiment. n=4-5 mice per group. Recipients only received a single dose of modified T cells, without cytokine support or vaccination. Due to a limit on T cell number, mice received 5-fold less T cells than compared to mice that received the presumably higher affinity Mesothelin₂₀₋₂₈-specific T cells in FIG. 6A-B. Thus, despite presumably lower affinity, a 5-fold lower number of Mesothelin₅₃₀₋₅₃₈-specific T cells confer similar efficacy in this orthotopic mouse model. The study associated with FIG. 6 involved testing the highest affinity TCRs, based on tetramer staining (clone 2 and clone 4); these T cells have some in vivo antitumor activity. Based on in vitro results in FIG. 4-5, the lower affinity TCRs may even prove more efficacious in vivo.

FIG. 7A illustrates the protocol for a study involving an immunocompetent mouse model of spontaneous pancreatic cancer. Pancreatic cancer produces a robust fibroinflammatory stroma response, and most of the tumor mass is not tumor cells, but instead immune suppressive hematopoietic and mesenchymal cells. Immunocompromised xenograft mouse models such as the NSG model (FIG. 6) fail to recapitulate the hallmark fibroinflammatory response, and therefore may be easier to treat with T cell therapies than human pancreatic cancer. Further, since mesothelin is expressed at low levels in the pleura, pericardium, and peritoneum, xenograft models using human TCRs reactive to human Mesothelin (not mouse mesothelin) will not permit testing for on-target off tumor toxicities. Parallel mouse TCRs specific to mesothelin were generated to assess safety and toxicity in a syngeneic and immunocompetent genetically engineered mouse model of pancreatic cancer which recapitulates many cardinal features of the human disease and immunotherapy response. FIG. 7A shows treatment regimens to test efficacy of parallel murine mesothelin specific TCR (1045) in a rigorous and highly aggressive syngeneic immunocompetent mouse model of spontaneous pancreatic cancer (referred to as KPC mice) in which tumor cells overexpress the target antigen mesothelin. The pancreas of KPC mice is imaged using high-resolution ultrasound (Vevo2100). Mice were enrolled to receive the initial dose of T cells based on advanced tumor burden (3-7 mm pancreatic tumor mass in diameter). Mice were preconditioned with cyclophosphamide to induce transient lymphodepletion. Five to six hours later, mice received T cells+recombinant human IL-2 on days 0, 2, 4, 6, and 8 post each T cell infusion. T cells were given every 2 weeks for a maximum of 3 doses. The top row shows KPC mice that received T cells only cohort (no vaccine). The middle row shows the cohort that received T cells combined with vaccine regimen #1. This regimen consists of agonistic anti-CD40 (mouse specific, clone FGK45), adjuvant Poly:IC which stimulates Type I interferons, and mesothelin peptide, which is a 9 amino acid peptide that is the sequence of the epitope in which the engineered T cells are reactive toward. The bottom row shows vaccine regimen #2 in which the sequencing of the vaccination components was changed, as indicated.

FIG. 7B illustrates the results of the study described in FIG. 7A. Percent survival is indicated on the y-axis while days is provided on the x-axis; untreated subjects are noted by a thin gray line, T cell treatment alone is referenced by a thick gray line, vaccine regimen #1 is a dashed line, and vaccine regiment #2 is referenced by a thick dark line. The adoptive transfer parallel murine T cells engineered to express a murine TCR specific to mouse mesothelin (1045) significantly prolonged mouse survival when combined with vaccine regimen #2. KPC mice were enrolled to receive the first T cell dose based on a large tumor burden in the pancreas (3-7 mm tumor mass in diameter). While the transfer of T cells alone moderated improvement, the transfer of T cells in combination with vaccination regimen #2 (see FIG. 7A) significantly prolonged survival. The sequencing of the vaccine components impacted the results observed. Vaccine regimen #2, wherein Poly:IC was administered with the first and third dose without anti-CD40, provided an unexpectedly superior benefit and significantly prolonged survival (dark solid line in graph).

FIG. 8A shows similar expansion of donor (Thy1.1+CD8+) engineered 1045 T cells on day 7 post vaccine regimen #1 (FIG. 7A) as compared to vaccine regimen #2 (FIG. 7A). This data shows that both vaccination strategies increase the frequency of infused T cells as compared to T cell only in circulation.

FIG. 8B shows that both vaccination regimens (FIG. 7A) increased Klrg1+engineered T cells, which are fully differentiated effector T cells in circulation. Tcf1, which is a memory/stem cell transcription factor, is maintained on engineered T cells following vaccination. These results demonstrate that the vaccination strategies are likely not impairing the longevity of engineered T cells following infusion.

FIG. 9A shows an experimental design to test the impact of knocking out TGFβR2 (Transforming Growth Factor Beta Receptor 2) using guide-specific RNA to TGFβR2 and CRISPR/Cas9 prior to the infusion of 1045 T cells in a highly aggressive and non-immunogenic orthotopic KPC mouse model.

Briefly, C57B16/J mice were surgically implanted into the pancreas with 1×10⁵ KPC unmodified primary tumor cells which were isolated from a KPC mouse with invasive and metastatic PDA. On day 5 post tumor implantation, mice received cyclophosphamide to create a homeostatic niche for the infused 1045 engineered T cells. Five to six hours later, tumor-bearing mice received 5×10⁶ 1045 T cells, or 1045 T cells that were rendered deficient in TGFβR2 using CRISPR/Cas9-based approach (referred to as TGFβR2 cKO). Additional cohorts received vaccine regimen #1. On day 12 post transfer, mice were euthanized, and tumor weights were measured.

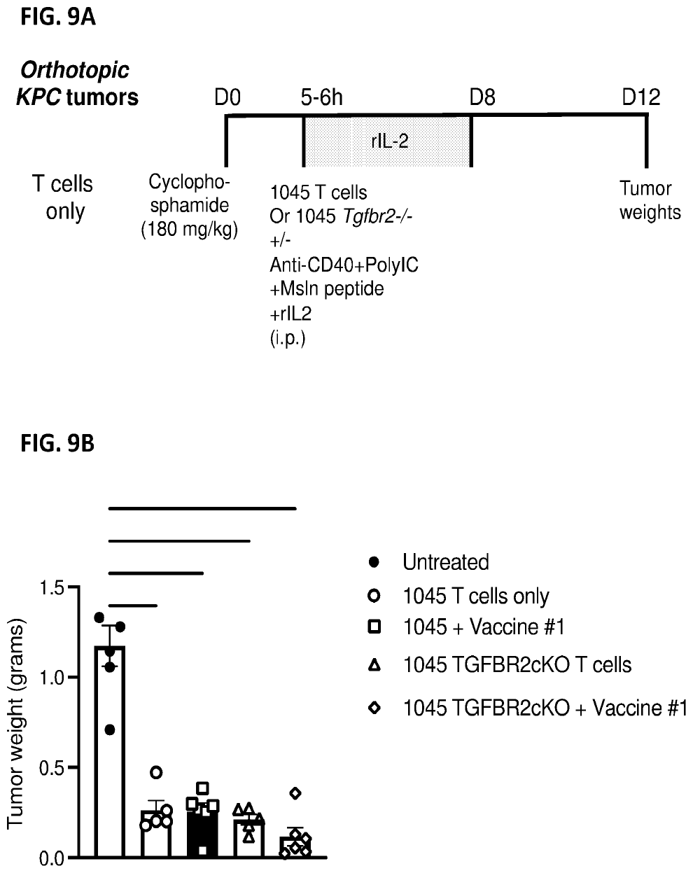

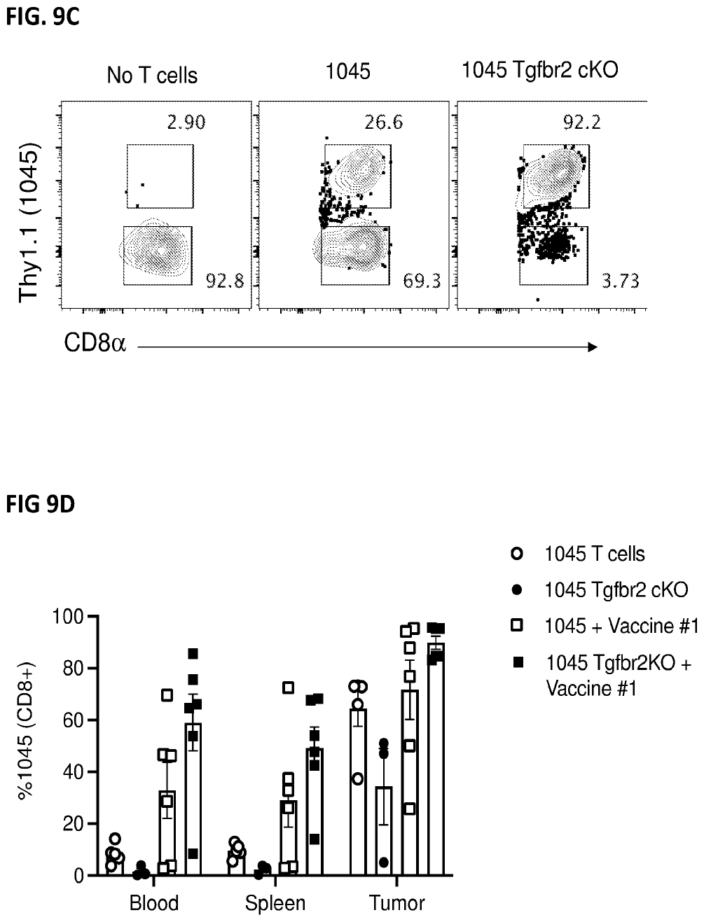

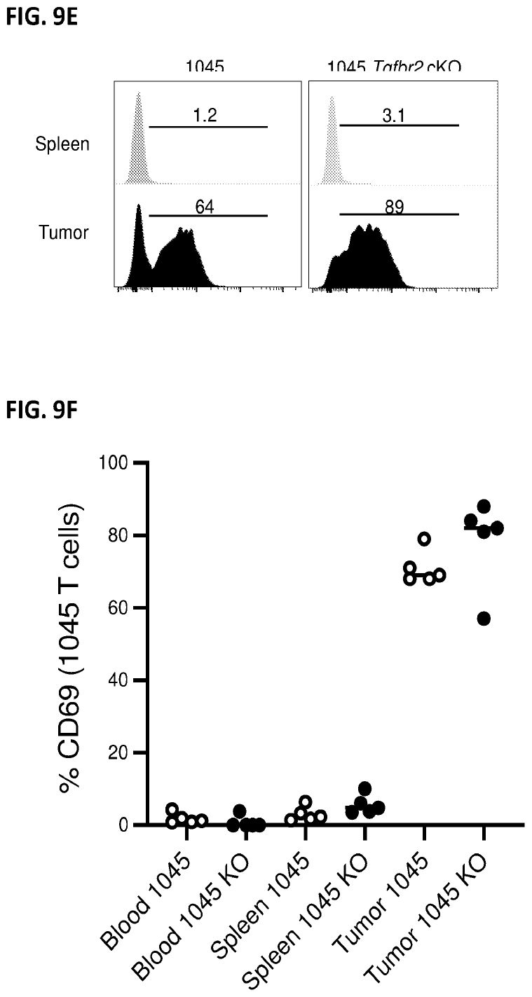

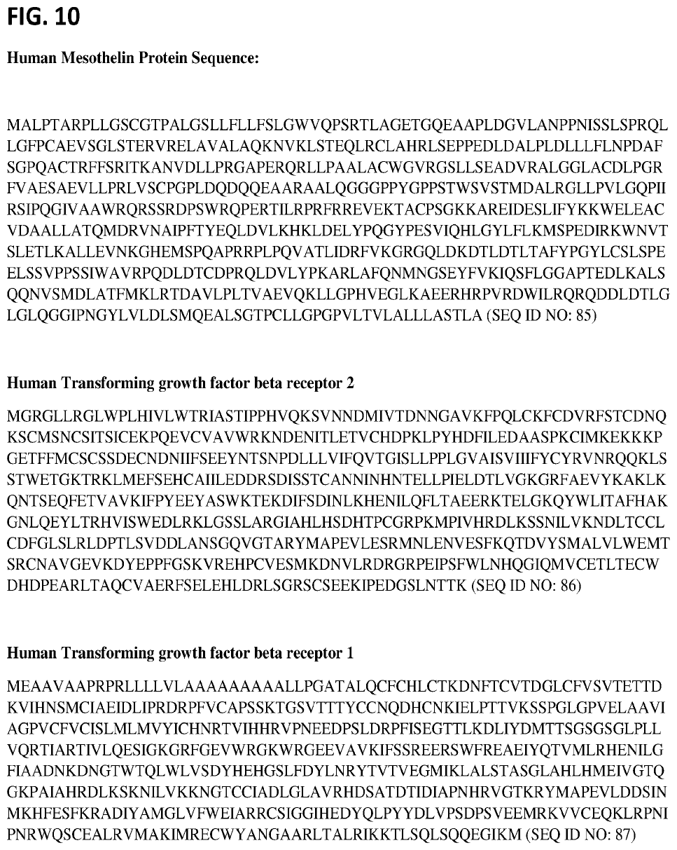

FIG. 9B shows tumor weights from experiments described in FIG. 9A. Each dot is an independent mouse. Representative of n=2-3 pooled independent experiments. ***, p<0.0001. ANOVA with a Tukey's posttest to correct for multiple comparisons.

FIG. 9C shows that knocking out TGFβR2 in engineered T cells increases donor T cell accumulation in tumors. Vaccine regimen #1 was included in both T cell infusions. Plots are gated on live, CD45+CD8+ T cells on day 7 post T cell infusion.

FIG. 9D is a bar graph illustrating that knock out of TGFβR2 in engineered T cells increases donor T cell accumulation in tumors post vaccination.

FIG. 9E illustrates that CD69 is upregulated in response to T cell receptor (TCR) signaling in recipients of T cells only (no vaccine) on day 7 post T cell transfer into pancreatic tumor bearing mice. The data suggest that abrogating TGFβR2 in TCR engineered T cells may promote antigen recognition in the tumor microenvironment.

FIG. 9F illustrates percentage of CD69 T cells determined via quantified analysis of multiple independent recipient mice from data in FIG. 9E. Overall, interfering with TGFβR2 (knockout (KO) 1045 cells) tends increase frequency of CD69 expression by tumor-infiltrating engineered T cells. Similar to FIG. 9E, the data evidence that interfering with TGFβ signaling can promote antigen recognition, and thus overcome immunosuppression in the tumor microenvironment.

FIG. 10 provides the amino acid sequences of human mesothelin, human transforming growth factor beta receptor 2, and human transforming growth factor beta receptor 1.

### DETAILED DESCRIPTION

This disclosure provides a mesothelin-specific binding protein, that is, a protein or polypeptide that binds to mesothelin (including a peptide or fragment thereof). In some embodiments, the mesothelin-specific binding protein binds to mesothelin (or a peptide or fragment thereof) complexed with an MHC. A mesothelin-specific binding protein may include a mesothelin-specific T cell receptor (TCR). The mesothelin-specific binding protein may be used in adoptive cell therapy (ACT). In some embodiments, the mesothelin-specific binding protein is sufficiently avid to mediate lysing of a tumor in an antigen-specific manner, but does not have sufficiently high affinity to result in off-tumor toxicity.

This disclosure also describes methods of using a mesothelin-specific binding protein, including uses in combination with other therapies. For example, a cell that overexpresses the mesothelin-specific binding protein may also overexpress a molecule that improves the immune response to mesothelin or to a tumor expressing mesothelin. A cell that overexpresses the mesothelin-specific binding protein may also overexpress a molecule that interferes with inhibitory receptor expression; a molecule that interferes with suppressive cytokine signaling; a molecule that renders T cells resistant to a program of T cell exhaustion and/or promotes resident memory; a chimeric costimulatory receptor; and/or an anti-tumor factor.

### Mesothelin

Mesothelin (also referred to herein as Msln or MSLN) is a self/tumor antigen that is overexpressed in several malignancies including pancreatic (Argani et al. Clin Cancer Res 7, 3862-3868 (2001), Hassan et al. J Clin Oncol 34, 4171-4179 (2016), Stromnes et al. Cancer Cell 28, 638-652 (2015)), ovarian (Coelho et al. Oncogenesis 9, 61 (2020)), lung (Thomas et al. Oncotarget 6, 11694-11703 (2015)), and breast (Tchou et al. Breast Cancer Res Treat 133, 799-804 (2012)) cancers. Due to its robust expression in malignancy, mesothelin is a promising target for immunotherapy (Pastan et al. Cancer Res 74, 2907-2912 (2014)); see also U.S. Publication No. 2018/0369280A1. Mesothelin-deficient mice have no phenotype indicating that that this gene is not essential for life. Mesothelin is immunogenic in humans with mesothelin-reactive T cell responses correlating with overall survival in pancreatic cancer patients (Thomas et al. J Exp Med 200, 297-306 (2004)).

### Adoptive Cell Therapy (ACT)

Adoptive cell therapy (ACT) involves, in various aspects, the ex vivo expansion and infusion of tumor-reactive cells (e.g., T cells) into cancer patients. In addition, the tumor-reactive cells (e.g., T cells) may further undergo genetic manipulation before being infused into cancer patients.

The adoptive transfer of T cells that express a tumor-reactive TCR have shown efficacy for some solid tumors (Chapuis et al. Nat Med 25, 1064-1072 (2019), Chapuis et al. Sci Transl Med 5, 174ra127 (2013), Johnson et al. Blood 114, 535-546 (2009), Kageyama et al. Clin Cancer Res 21, 2268-2277 (2015), Morgan et al. Science 314, 126-129 (2006), Rapoport et al. Nat Med 21, 914-921 (2015), Robbins et al. Clin Cancer Res 21, 1019-1027 (2015), Tran et al. N Engl J Med 375, 2255-2262 (2016), Tran et al. Science 344, 641-645 (2014), Yee et al. Proc Natl Acad Sci U S A 99, 16168-16173 (2002), Zacharakis et al. Nat Med 24, 724-730 (2018)). Historically, this therapy was developed for melanoma with the adoptive transfer of ex vivo expanded polyclonal tumor-infiltrating lymphocytes (TILs) (Hinrichs et al. Immunol Rev 257, 56-71 (2014)). T cells that confer antitumor activity are often specific to mutated neoepitopes (Tran et al. N Engl J Med 375, 2255-2262 (2016), Tran et al. Science 344, 641-645 (2014), Zacharakis et al. Nat Med 24, 724-730 (2018)), as well as tissue-associated antigens (Chandran et al. Immunol Rev 290, 127-147 (2019)). In vitro expanded T cells specific to virus epitopes can therapeutically target virally-induced malignancies including cervical cancer and Merkel cell carcinoma (Paulson et al. Nat Commun 9, 3868 (2018), Stevanovic et al. J Clin Oncol 33, 1543-1550 (2015)). Transfer of neoantigen-enriched TILs can also cause tumor regressions in some epithelial malignancies (Tran et al. Science 344, 641-645 (2014), Zacharakis et al. Nat Med 24, 724-730 (2018)). However, TIL therapy is highly personalized and not ideal for some malignancies that lack endogenous tumor-reactive T cells. Therefore, genetic modification of a patients' own T cells to express a tumor-reactive TCR of a defined specificity and functionality is a promising alternative. TCR-engineered T cells (TCR-T) are expanded with specific antigen and various cytokines in vitro and then infused back into patients following a lymphodepletion regimen (Stromnes et al. Immunol Rev 257, 145-164 (2014)), similar to a chimeric-antigen receptor (CAR)-T cells approach. TCR-T cells are clonal and express a TCR with defined specificity and reactivity. There is substantial time and effort to clone, screen, validate and select clinical TCRs (Rollins et al. Curr Protoc Immunol 129, e97 (2020)). Therefore, TCRs reactive to commonly overexpressed self/tumor antigens including Mesothelin (Stromnes et al. Cancer Cell 28, 638-652 (2015)), WT-1 (Chapuis et al. Nat Med 25, 1064-1072 (2019), Chapuis et al. Sci Transl Med 5, 174ra127 (2013)), NY-ESO-1 (Rapoport et al. Nat Med 21, 914-921 (2015)), MART-1 (Chodon et al. Clin Cancer Res 20, 2457-2465 (2014), van den Berg et al. Mol Ther 23, 1541-1550 (2015)), Vestigial-1 (Bradley et al. Nat Commun 11, 5332 (2020)), as well as public neoantigens such as mutant KRAS (Tran et al. N Engl J Med 375, 2255-2262 (2016), Chandran et al. Immunol Rev 290, 127-147 (2019), Klebanoff et al. J Exp Med 215, 5-7 (2018), Wang et al. Cancer Immunol Res 4, 204-214 (2016)), may be particularly useful because a single TCR could treat multiple individuals.

Previously, most researchers in the field selected TCRs of high affinity for targeting cancer with engineered T cells. It was assumed that a higher affinity TCR would provide superior anti-tumor activity by increasing T cell recognition of antigen (for example, peptide: HLA complexes) on the tumor cell surface. However, efforts to enhance the affinity of TCRs to self/tumor antigens have resulted in lethal toxicity.

Higher affinity TCRs require lower amounts of antigen to elicit a functional response and thus are sought after for clinical translation. However, there is also concern when targeting self/tumor antigens with high affinity TCRs because of the potential for off-tumor, on-target toxicity. A MAGE-A3-specific TCR, which was derived from HLA-A*02:01 transgenic mice, caused lethal neurological toxicity in some patients, due to recognition of a brain peptide derived from the MAGE family (Morgan et al. J Immunother 36, 133-151 (2013)). Another TCR specific to MAGE-A3 was affinity-enhanced by mutating amino acid residues in CDR2 and CDR3 and caused fatal toxicity due to TCR cross-reactivity to a completely different self-peptide expressed in the heart (Linette et al. Blood 122, 863-871 (2013)). A MART-1 TCR, which was not affinity-enhanced, caused lethal toxicity in a single patient (van den Berg et al. Mol Ther 23, 1541-1550 (2015)). Similarly, severe toxicities, albeit nonlethal, were also observed with a MART-1 TCR transfer in combination with DC vaccination (Chodon et al. Clin Cancer Res 20, 2457-2465 (2014)). In addition, increased risk for toxicity, a high affinity TCR may also promote T cell exhaustion, which is a differentiation state of tumor-reactive T cells due that is dependent on chronic TCR signaling. Therefore, there may be particular advantages to incorporating moderately avid tumor-reactive TCRs for cell therapy.

Although, as noted above, a tumor-reactive TCR may be similar to a chimeric antigen receptor (CAR)-based therapy, CARs can only recognize cell surface proteins. There are few antigens that are highly expressed on the cell surface of a tumor cell that is safe to target with a cell-based therapy. Additionally, there is no proof-of-principle in the clinic that a synthetic CAR T cell therapy can eradicate solid tumors in patients. In contrast, T cells which express T cell receptors (TCRs) have repeatedly demonstrated the capability to eradicate large, bulky solid tumors in patients. Further, modifying T cells to express a tumor-reactive TCR permits therapeutically targeting intracellular antigens, which comprise the majority of proteins expressed in the cell.

Thus, in one aspect, as further described herein, this disclosure describes mesothelin-reactive TCRs that are sufficiently avid to lyse tumor in an antigen-specific manner.

### T Cell Receptor and T Cell Receptor Structure

The T cell receptor (TCR) typically includes two different protein chains. In humans, in most T cells, the TCR includes an alpha (α) chain and a beta (β) chain.

TCR gene segments rearrange during T cell development to form complete variable regions or variable domains (also referred to herein as V_{α} and Vβ). The arrangement of the gene segments resembles that of the immunoglobulin loci, with separate variable (V), diversity (D), joining (J) gene segments, and constant (C) genes. The TCR α chain is generated by VJ recombination, whereas the β chain is generated by VDJ recombination.

The TCRα locus (which is on chromosome 14) includes 70-80 V_{α} gene segments and a cluster of 61 J_{α} gene segments, located a considerable distance from the V_{α} gene segments. The J_{α} gene segments are followed by a single C gene, which encodes the constant domain, a hinge domain, and the transmembrane and cytoplasmic regions.

The TCRβ locus (which is on chromosome 7) includes 52 V_{β} gene segments, two separate clusters each containing a single D gene segment, six or seven J gene segments, and a single C gene. Each TCRβ C gene encodes the constant domain, the hinge, the transmembrane region, and the cytoplasmic region.

Each variable region (Vα and Vβ) has three loops called complementary determining regions (CDRs) that directly interact with a peptide-MHC complex (also referred to as pMHC). Structural studies have shown that CDR3 loops usually present the most discriminative interactions with peptides, meanwhile CDR2 loops interact mainly with the MHC, and CDR1 loops tend to present soft interactions with both peptide and MHC (Lanzarotti et al. Front Immunol 10, 2080 (2019), Garcia et al. Cell 122, 333-336 (2005)). CDR1 and CDR2 loop sequences are constant for each type of chain and are therefore referred to as “germline derived,” whereas the CDR3 loops vary in an almost unlimited fashion and largely dictate the TCR specificity for peptide (Garcia et al. Cell 122, 333-336 (2005)). In addition, single-variable-domain TCR (svd TCR) that include only the variable domain of the β chain (Vβ) may bind pMHC tetramers selectively and trigger T cells in much the same manner as full TCRs (Oh et al. Sci Rep 9, 17291 (2019)).

### Mesothelin-Specific Binding Proteins and T Cell Receptors

In one aspect, this disclosure describes mesothelin-specific binding proteins including, for example, mesothelin-specific T cell receptors (TCRs).

In some embodiments, a mesothelin-specific binding protein may include a TCR Vα CDR3. In some embodiments, a mesothelin-specific binding protein may include a TCR Vβ CDR3. In some embodiments, a mesothelin-specific binding protein may include a TCR α-chain CDR3 and a TCR β-chain CDR3. In some embodiments, a mesothelin-specific binding protein may include a TCR α-chain variable (Vα) domain. In some embodiments, a mesothelin-specific binding protein may include a TCR β-chain variable (Vβ) domain. In some embodiments, a mesothelin-specific binding protein may include a TCR α-chain variable (Vα) domain and a TCR β-chain variable (Vβ) domain. When the mesothelin-specific binding protein is a mesothelin-specific T cell receptor, it may include a TCR α-chain variable (Vα) domain, a TCR β-chain variable (Vβ) domain, a TCR α-chain constant domain, and a TCR β-chain constant domain.

In some embodiments, a mesothelin-specific binding protein binds a mesothelin-tetramer (for example, a fluorescently-labeled Msln₂₀₋₂₈-HLA-A2 tetramer or a fluorescently-labeled Msln₅₃₀₋₅₃₉: HLA-A2 tetramer). In various aspects, the mesothelin-specific binding protein (e.g., mesothelin-specific T cell receptor) binds a mesothelin-tetramer with a K_{d} of less than 10⁻⁸ M, less than 10⁻⁹ M, less than 10⁻¹⁰ M, less than 10⁻¹¹ M, less than 10⁻¹² M, or less than 10⁻¹³ M. In some embodiments, a mesothelin-specific binding protein binds a mesothelin-tetramer with a K_{d} of at least 10⁻⁷ M. Binding affinity may be characterized using any of a number of routine laboratory assays, such as enzyme-linked immunosorbent assay (ELISA) or Surface Plasmon Resonance (SPR) techniques (analyzed on a BIAcore instrument) (Liljeblad et al., Glyco J 17, 323-329 (2000)), as well as traditional binding assays (Heeley, Endocr Res 28, 217-229 (2002)) (see also, e.g., Scatchard, et al., Ann. N. Y. Acad. Sci. 57:660, 1949; and U.S. Pat. Nos. 5,283,173, 5,468,614). The mesothelin-specific binding protein binds mesothelin with a greater affinity than other, unrelated proteins (e.g., the extent of binding to an unrelated protein (e.g., egg white lysozyme) is less than about 10% of the binding to mesothelin as measured, e.g. by SPR.).

In various aspects, the mesothelin-specific binding protein binds a mesothelin-tetramer with intermediate affinity, which, in various aspects, results in superior tumor lysis. Optionally, the mesothelin-specific binding proteins binds a mesothelin-tetramer in the presence of CD8 such that >2-fold mean fluorescence intensity (MFI) is observed as compared to TCR-negative control T cells. Alternatively or in addition, the mesothelin-specific binding protein optionally is unable to bind tetramer in the absence of CD8, as observed with MSLN₅₃₀₋₅₃₈ binding peptides described herein. Alternatively or in addition, the mesothelin-specific binding protein optionally produces TCR signaling molecules (e.g., Nur77) and effector cytokines (e.g., IFNγ) and activation markers (e.g., CD25, CD69, or CD137) only following specific antigen encounter.

### MSLN₂₀₋₂₈—Specific Binding Proteins

In some embodiments, the mesothelin-specific binding protein (including, for example, a TCR) is reactive to (i.e., binds) amino acids 20-28 of human mesothelin in the context of HLA-A201.

In some embodiments, a mesothelin-specific binding protein reactive to amino acids 20-28 of human mesothelin includes a TCR constant region. In some embodiments, the TCR constant region includes a cysteine modification to induce preferential pairing of the TCR constant regions.

In some embodiments, a mesothelin-specific binding protein reactive to amino acids 20-28 of human mesothelin includes a TCRα CDR3 of one of the clones described in Example 2 of this disclosure. In some embodiments, a mesothelin-specific binding protein reactive to amino acids 20-28 of human mesothelin includes a TCRβ CDR3 of one of the clones described in Example 2 of this disclosure. In some embodiments, a mesothelin-specific binding protein reactive to amino acids 20-28 of human mesothelin includes both a TCRα CDR3 of one of the clones described in Example 2 and TCRβ CDR3 of one of the clones described in Example 2; the TCRα CDR3 and the TCRβ CDR3 may be from the same clone.

In some embodiments, a mesothelin-specific binding protein reactive to amino acids 20-28 of human mesothelin includes a TCRα CDR3 having a peptide sequence of CAASGNTDKLIF (SEQ ID NO:3).

In some embodiments, a mesothelin-specific binding protein reactive to amino acids 20-28 of human mesothelin includes a TCRα CDR3 having a peptide sequence of CAFYMDSNYQLIW (SEQ ID NO:4).

In some embodiments, a mesothelin-specific binding protein reactive to amino acids 20-28 of human mesothelin includes a TCRα CDR3 having a peptide sequence of CAVIPNNNARLMF (SEQ ID NO:5).

In some embodiments, a mesothelin-specific binding protein reactive to amino acids 20-28 of human mesothelin includes a TCRβ CDR3 having a peptide sequence of CASRPGWSYEQYF (SEQ ID NO:6).

In some embodiments, a mesothelin-specific binding protein reactive to amino acids 20-28 of human mesothelin includes a TCRβ CDR3 having a peptide sequence of CASSEWTAEQYF (SEQ ID NO:7).

In some embodiments, a mesothelin-specific binding protein reactive to amino acids 20-28 of human mesothelin includes a TCRβ CDR3 having a peptide sequence of CASGQGTEAFF (SEQ ID NO:8).

In some embodiments, a mesothelin-specific binding protein reactive to amino acids 20-28 of human mesothelin includes a TCRα CDR3 having a peptide sequence of CAASGNTDKLIF (SEQ ID NO:3) and a TCRβ CDR3 having a peptide sequence of CASRPGWSYEQYF (SEQ ID NO:6).

In some embodiments, a mesothelin-specific TCR reactive to amino acids 20-28 of human mesothelin includes a TCRα CDR3 having a peptide sequence of CAFYMDSNYQLIW (SEQ ID NO:4) and a TCRβ CDR3 having a peptide sequence of CASSEWTAEQYF (SEQ ID NO:7).

In some embodiments, a mesothelin-specific TCR reactive to amino acids 20-28 of human mesothelin includes a TCRα CDR3 having a peptide sequence of CAVIPNNNARLMF (SEQ ID NO:5) and a TCRβ CDR3 having a peptide sequence of CASGQGTEAFF (SEQ ID NO:8).

In some embodiments, a mesothelin-specific binding protein reactive to amino acids 20-28 of human mesothelin includes a Valpha peptide sequence of one of the clones described in Example 2 of this disclosure. In some embodiments, a mesothelin-specific binding protein reactive to amino acids 20-28 of human mesothelin includes a Vbeta peptide sequence of one of the clones described in Example 2 of this disclosure. In some embodiments, a mesothelin-specific binding protein reactive to amino acids 20-28 of human mesothelin includes both the Valpha peptide sequence and a Vbeta peptide sequence from a clone described in Example 2; the Valpha peptide sequence and a Vbeta peptide sequence may be from the same clone. In some embodiments, a mesothelin-specific binding protein reactive to amino acids 20-28 of human mesothelin includes the peptide sequence of Vα TRAV29/DV5*01 and TRAJ34*01. In some embodiments, a mesothelin-specific binding protein reactive to amino acids 20-28 of human mesothelin includes the peptide sequence of Vα TRAV24*01 and TRAJ33*01. In some embodiments, a mesothelin-specific binding protein reactive to amino acids 20-28 of human mesothelin includes the peptide sequence of Vα TRAV8-6*022 and TRAJ31*01.

In some embodiments, a mesothelin-specific binding protein reactive to amino acids 20-28 of human mesothelin includes the peptide sequence of Vβ TRBV2*01; TRBJ2-7*01 and TRBD1*01. In some embodiments, a mesothelin-specific binding protein reactive to amino acids 20-28 of human mesothelin includes the peptide sequence of Vβ TRBV6-1*01; TRBJ2-3*01; and TRBD1*01. In some embodiments, a mesothelin-specific binding protein reactive to amino acids 20-28 of human mesothelin includes the peptide sequence of Vβ TRBV4-2*01; TRBJ1-1*01; and TRBD1*01.

In some embodiments, a mesothelin-specific binding protein reactive to amino acids 20-28 of human mesothelin includes a peptide sequence of Table 1. Table 2, or Table 3. In some embodiments, a mesothelin-specific binding protein reactive to amino acids 20-28 of human mesothelin includes a Valpha peptide sequence of Table 1, Table 2, or Table 3. In some embodiments, a mesothelin-specific binding protein reactive to amino acids 20-28 of human mesothelin comprises the CDR sequences present in a Valpha peptide sequence of Table 1, Table 2, or Table 3. CDR1 is located at about positions 27-38 in the Valpha peptide and CDR2 is located at about positions 56-65 of the Valpha peptide. See, e.g., SEQ ID NOs: 88, 89, 92, 93, 96, and 97. In some embodiments, a mesothelin-specific binding protein reactive to amino acids 20-28 of human mesothelin includes a Vbeta peptide sequence of Table 1, Table 2, or Table 3. In some embodiments, a mesothelin-specific binding protein reactive to amino acids 20-28 of human mesothelin comprises the CDR sequences present in a Vbeta peptide sequence of Table 1, Table 2, or Table 3 CDR1 is located at about positions 27-38 in the Vbeta peptide and CDR2 is located at about positions 56-65 of the Vbeta peptide. See, e.g., SEQ ID NOs: 90, 91, 94, 95, 98, and 99. In some embodiments, a mesothelin-specific binding protein reactive to amino acids 20-28 of human mesothelin includes a Valpha-Valpha constant peptide sequence of Table 1. Table 2, or Table 3. In some embodiments, a mesothelin-specific binding protein reactive to amino acids 20-28 of human mesothelin includes a Vbeta-Vbeta constant peptide sequence of Table 1, Table 2, or Table 3. In some embodiments, a mesothelin-specific binding protein reactive to amino acids 20-28 of human mesothelin includes a Vbeta-Vbeta constant of Table 1, Table 2, or Table 3 and a Valpha-Valpha constant peptide sequence of Table 1. Table 2, or Table 3. In some embodiments, a mesothelin-specific binding protein reactive to amino acids 20-28 of human mesothelin includes a Vbeta-Vbeta constant-P2A-Valpha-Valpha constant peptide sequence of Table 1, Table 2, or Table 3.

In various aspects, the mesothelin-specific binding protein comprises a Valpha amino acid sequence of SEQ ID NO: 24, 33, or 43. In various aspects, the mesothelin-specific binding protein comprises a Valpha amino acid sequence of SEQ ID NO: 26, 36, or 46. In various aspects, the mesothelin-specific binding protein comprises a Valpha amino acid sequence of SEQ ID NO: 24 and a Vbeta amino acid sequence of SEQ ID NO: 26, a Valpha amino acid sequence of SEQ ID NO: 33 and a Vbeta amino acid sequence of SEQ ID NO: 36, or a Valpha amino acid sequence of SEQ ID NO: 43 and a Vbeta amino acid sequence of SEQ ID NO: 46. In various aspects, the mesothelin-specific binding protein comprises variable region sequences comprising an amino acid sequence at least 90% identical (e.g., at least 95% identical) to the sequences set forth in SEQ ID NO: 24, 33, or 43 and/or SEQ ID NO: 26, 36, or 46, optionally comprising substitutions (e.g., conservative substitutions) outside of the CDR3 region.

In some embodiments, a mesothelin-specific binding protein reactive to amino acids 20-28 of human mesothelin includes a Valpha domain that has at least 75%, at least 80%, at least 85%, at least 90%, at least 95%, at least 96%, at least 97%, at least 98%, or at least 99% sequence identity to the amino acid sequence of a Valpha-Valpha constant peptide sequence of Table 1, Table 2, or Table 3 (SEQ ID NO: 25, 35, or 45). In some embodiments, none of the CDRs of the mesothelin-specific binding protein reactive to amino acids 20-28 of human mesothelin include a mutation relative to the CDRs of the Valpha-Valpha constant peptide sequence of Table 1, Table 2, or Table 3. In some embodiments, the TCRα CDR3 does not include a mutation relative to the corresponding TCRα CDR3 of the Valpha-Valpha constant peptide sequence of Table 1, Table 2, or Table 3.

In some embodiments, a mesothelin-specific binding protein reactive to amino acids 20-28 of human mesothelin includes a Vbeta domain that has at least 75%, at least 80%, at least 85%, at least 90%. at least 95%, at least 96%, at least 97%, at least 98%, or at least 99% sequence identity to the amino acid sequence of a Vbeta-Vbeta constant peptide sequence of Table 1, Table 2, or Table 3 (SEQ ID NO: 28, 38, or 48). In some embodiments, none of the CDR sequences of the mesothelin-specific binding protein reactive to amino acids 20-28 of human mesothelin include a mutation relative to the CDRs of the Vbeta-Vbeta constant peptide sequence of Table 1, Table 2, or Table 3. In some embodiments, the TCRβ CDR3 amino acid sequence does not include a mutation relative to the corresponding TCRβ CDR3 amino acid sequence of the Vbeta-Vbeta constant peptide sequence of Table 1, Table 2, or Table 3.

In various embodiments, the mesothelin-specific binding protein may include variant polypeptide species that have one or more amino acid substitutions, insertions, or deletions in the amino acid sequence relative of a mesothelin-specific binding protein presented herein, provided that the mesothelin-specific binding protein retains its ability to bind to a mesothelin-tetramer (for example, a fluorescently-labeled Msln₂₀₋₂₈-HLA-A2 tetramer), optionally with a K_{d} of less than 10⁻⁸ M, less than 10⁻⁹ M, less than 10⁻¹⁰ M, less than 10⁻¹¹ M, less than 10⁻¹² M, or less than 10⁻¹³ M.

In another aspect, this disclosure describes an isolated polynucleotide molecule encoding a mesothelin-specific binding protein reactive to amino acids 20-28 of human mesothelin. In some embodiments, the isolated polynucleotide molecule includes a codon optimized sequence of a mesothelin-specific binding protein reactive to amino acids 20-28 of human mesothelin, as described herein. In some embodiments, the disclosure provides an isolated polynucleotide molecule comprising a nucleotide sequence of Table 1 (one or more of SEQ ID NOs: 15-22), Table 2 (one or more of SEQ ID NOs: 30-32), and/or Table 3 (one or more of SEQ ID NOs: 40-42).

### MSLN₅₃₀₋₅₃₈—Specific Binding Proteins

In some embodiments, a mesothelin-specific binding protein (including, for example, a TCR) is reactive to (i.e., binds) amino acids 530-538 of human mesothelin in the context of HLA-A201.

In some embodiments, a mesothelin-specific binding protein TCR reactive to amino acids 530-538 of human mesothelin includes a TCR constant region. In some embodiments, the TCR constant region includes a cysteine modification to induce preferential pairing of the TCR constant regions.

In some embodiments, a mesothelin-specific binding protein reactive to amino acids 530-538 of human mesothelin includes a TCRα CDR3 of one of the clones described in Example 2 of this disclosure. In some embodiments, a TCRβ of a mesothelin-specific binding protein reactive to amino acids 530-538 of human mesothelin includes a TCRα CDR3 of one of the clones described in Example 2 of this disclosure. In some embodiments, a TCRα of a mesothelin-specific binding protein reactive to amino acids 530-538 of human mesothelin includes both a TCRα CDR3 of one of the clones described in Example 2 and TCRβ CDR3 of one of the clones described in Example 2; the TCRα CDR3 and the TCRβ CDR3 may be from the same clone.

In some embodiments, a mesothelin-specific binding protein reactive to amino acids 530-538 of human mesothelin includes a TCRα CDR3 having a peptide sequence of CAYLGTGTYKYIF (SEQ ID NO:9).

In some embodiments, a mesothelin-specific binding protein reactive to amino acids 530-538 of human mesothelin includes a TCRα CDR3 having a peptide sequence of CAGGMESGGGADGLTF (SEQ ID NO:10).

In some embodiments, a mesothelin-specific binding protein reactive to amino acids 530-538 of human mesothelin includes a TCRα CDR3 having a peptide sequence of CALDTGFQKLVF (SEQ ID NO:11).

In some embodiments, a mesothelin-specific binding protein reactive to amino acids 530-538 of human mesothelin includes a TCRβ CDR3 having a peptide sequence of CASSSGGLGYTF (SEQ ID NO:12).

In some embodiments, a mesothelin-specific binding protein reactive to amino acids 530-538 of human mesothelin includes a TCRβ CDR3 having a peptide sequence of CASTSTGGLKNTEAFF (SEQ

ID NO:13).

In some embodiments, a mesothelin-specific binding protein reactive to amino acids 530-538 of human mesothelin includes a TCRβ CDR3 having a peptide sequence of CASSSLGDRNTEAFF (SEQ ID NO:14).

In some embodiments, a mesothelin-specific binding protein reactive to amino acids 530-538 of human mesothelin includes a TCRα CDR3 having a peptide sequence of CAYLGTGTYKYIF (SEQ ID NO:9) and a TCRβ CDR3 having a peptide sequence of CASSSGGLGYTF (SEQ ID NO:12).

In some embodiments, a mesothelin-specific binding protein reactive to amino acids 530-538 of human mesothelin includes a TCRα CDR3 having a peptide sequence of CAGGMESGGGADGLTF (SEQ ID NO:10) and a TCRβ CDR3 having a peptide sequence of CASTSTGGLKNTEAFF (SEQ ID NO:13).

In some embodiments, a mesothelin-specific binding protein reactive to amino acids 530-538 of human mesothelin includes a TCRα CDR3 having a peptide sequence of CALDTGFQKLVF (SEQ ID NO:11) and a TCRβ CDR3 having a peptide sequence of CASSSLGDRNTEAFF (SEQ ID NO:14).

In some embodiments, a mesothelin-specific binding protein reactive to amino acids 530-538 of human mesothelin includes the Valpha peptide sequence of one of the clones described in Example 2 of this disclosure. In some embodiments, a mesothelin-specific binding protein reactive to amino acids 530-538 of human mesothelin includes a Vbeta peptide sequence of one of the clones described in Example 2 of this disclosure. In some embodiments, a mesothelin-specific binding protein reactive to amino acids 530-538 of human mesothelin includes both the Valpha peptide sequence and a Vbeta peptide sequence from a clone described in Example 2; the Valpha peptide sequence and a Vbeta peptide sequence may be from the same clone.

In some embodiments, a mesothelin-specific binding protein reactive to amino acids 530-538 of human mesothelin includes Vα TRAV38-1*04 and TRAJ40*01. In some embodiments, a mesothelin-specific binding protein reactive to amino acids 530-538 of human mesothelin includes Vα TRAV38-2/DV8*01 and TRAJ40*01. In some embodiments, a mesothelin-specific binding protein reactive to amino acids 530-538 of human mesothelin includes Vα TRAV27*03 and TRAJ45*01. In some embodiments, a mesothelin-specific binding protein reactive to amino acids 530-538 of human mesothelin includes Vα TRAV9-2*01 and TRAJ8*01.

In some embodiments, a mesothelin-specific binding protein reactive to amino acids 530-538 of human mesothelin includes Vβ TRBV27*01 and TRBJ2-6*01. In some embodiments, a mesothelin-specific binding protein reactive to amino acids 530-538 of human mesothelin includes Vβ TRBV7-9*01; TRBJ1-1*01 and TRBD1*01. In some embodiments, a mesothelin-specific binding protein reactive to amino acids 530-538 of human mesothelin includes Vβ TRBV27*01; TRBJ1-1*01; and TRBD1*01.

In some embodiments, a mesothelin-specific binding protein reactive to amino acids 530-538 of human mesothelin includes a peptide sequence of Table 4, Table 5, or Table 6. In some embodiments, a mesothelin-specific binding protein reactive to amino acids 530-538 of human mesothelin includes a Valpha peptide sequence of Table 4, Table 5, or Table 6. In some embodiments, a mesothelin-specific binding protein reactive to amino acids 530-538 of human mesothelin comprises CDR sequences present in a Valpha peptide sequence of Table 4, Table 5, or Table 6. CDR1 is located at about positions 27-38 in the Valpha peptide and CDR2 is located at about positions 56-65 of the Valpha peptide. See, e.g., SEQ ID NOs: 100, 101, 104, 105, 106, and 107. In some embodiments, a mesothelin-specific binding protein reactive to amino acids 530-538 of human mesothelin includes a Vbeta peptide sequence of Table 4, Table 5, or Table 6. In some embodiments, a mesothelin-specific binding protein reactive to amino acids 530-538 of human mesothelin comprises CDR sequences present in a Vbeta peptide sequence of Table 4, Table 5, or Table 6. CDR1 is located at about positions 27-38 in the Vbeta peptide and CDR2 is located at about positions 56-65 of the Vbeta peptide. See, e.g., SEQ ID NOs: 102, 103, 108, and 109. In some embodiments, a mesothelin-specific binding protein reactive to amino acids 530-538 of human mesothelin includes a Valpha-Valpha constant peptide sequence of Table 4, Table 5, or Table 6. In some embodiments, a mesothelin-specific binding protein reactive to amino acids 530-538 of human mesothelin includes a Vbeta-Vbeta constant peptide sequence of Table 4, Table 5, or Table 6. In some embodiments, a mesothelin-specific binding protein reactive to amino acids 530-538 of human mesothelin includes a Vbeta-Vbeta constant of Table 4, Table 5, or Table 6 and a Valpha-Valpha constant peptide sequence of Table 4, Table 5, or Table 6. In some embodiments, a mesothelin-specific binding protein reactive to amino acids 530-538 of human mesothelin includes a Vbeta-Vbeta constant-P2A-Valpha-Valpha constant peptide sequence of Table 4, Table 5, or Table 6.

In various aspects, the mesothelin-specific binding protein comprises a Valpha amino acid sequence of SEQ ID NO: 58, 68, or 78. In various aspects, the mesothelin-specific binding protein comprises a Valpha amino acid sequence of SEQ ID NO: 61, 71, or 81. In various aspects, the mesothelin-specific binding protein comprises a Valpha amino acid sequence of SEQ ID NO: 58 and a Vbeta amino acid sequence of SEQ ID NO: 61, a Valpha amino acid sequence of SEQ ID NO: 68 and a Vbeta amino acid sequence of SEQ ID NO: 71, or a Valpha amino acid sequence of SEQ ID NO: 78 and a Vbeta amino acid sequence of SEQ ID NO: 81. In various aspects, the mesothelin-specific binding protein comprises variable region sequences comprising an amino acid sequence at least 90% identical (e.g., at least 95% identical) to the sequences set forth in SEQ ID NO: 58, 68, or 78 and/or SEQ ID NO: 61. 71, or 81, optionally comprising substitutions (e.g., conservative substitutions) outside of the CDR3 region.

In some embodiments, a mesothelin-specific binding protein reactive to amino acids 530-538 of human mesothelin includes a Valpha domain that has at least 75%, at least 80%, at least 85%, at least 90%, at least 95%, at least 96%, at least 97%, at least 98%, or at least 99% sequence identity to the amino acid sequence of a Valpha-Valpha constant peptide sequence of Table 4, Table 5, or Table 6 (SEQ ID NO: 60, 70, or 80). In some embodiments, none of the CDR sequences of the mesothelin-specific binding protein reactive to amino acids 530-538 of human mesothelin include a mutation relative to the CDRs of the Valpha-Valpha constant peptide sequence of Table 4, Table 5, or Table 6. In some embodiments, the TCRα CDR3 amino acid sequence does not include a mutation relative to the corresponding TCRα CDR3 amino acid sequence of the Valpha-Valpha constant peptide sequence of Table 4, Table 5, or Table 6.

In various embodiments, a mesothelin-specific binding protein reactive to amino acids 530-538 of human mesothelin includes a Vbeta domain that has at least 75%, at least 80%, at least 85%, at least 90%. at least 95%, at least 96%, at least 97%, at least 98%, or at least 99% sequence identity to the amino acid sequence of a Vbeta-Vbeta constant peptide sequence of Table 4, Table 5, or Table 6 (SEQ ID NO: 63, 73, or 83). In some embodiments, none of the CDRs of the mesothelin-specific binding protein reactive to amino acids 530-538 of human mesothelin include a mutation relative to the CDRs of the Vbeta-Vbeta constant peptide sequence of Table 4, Table 5, or Table 6. In some embodiments, the TCRβ CDR3 amino acid sequence does not include a mutation relative to the corresponding TCRβ CDR3 amino acid sequence of the Vbeta-Vbeta constant peptide sequence of Table 4, Table 5, or Table 6.

In various embodiments, the mesothelin-specific binding protein may include variant polypeptide species that have one or more amino acid substitutions, insertions, or deletions in the amino acid sequence relative of a mesothelin-specific binding protein presented herein, provided that the mesothelin-specific binding protein retains its ability to bind to a mesothelin-tetramer (for example, a fluorescently-labeled Msln530 538-HLA-A2 tetramer), optionally with a K_{d} of less than 10⁻⁸ M, less than 10⁻⁹ M, less than 10⁻¹⁰ M, less than 10⁻¹¹ M, less than 10⁻¹² M, or less than 10⁻¹³ M.

In some embodiments, the isolated polynucleotide molecule includes a codon optimized sequence of a mesothelin-specific binding protein reactive to amino acids 530-538 of human mesothelin, as described herein.

In another aspect, this disclosure describes an isolated polynucleotide molecule encoding a mesothelin-specific binding protein reactive to amino acids 530-538 of human mesothelin. In some embodiments, the isolated polynucleotide molecule includes a codon optimized sequence of a mesothelin-specific binding protein reactive to amino acids 530-538 of human mesothelin, as described herein. In some embodiments, the disclosure provides an isolated polynucleotide molecule comprising a nucleotide sequence of Table 4 (one or more of SEQ ID NOs: 50-57), Table 5 (one or more of SEQ ID NOs: 65-67), or Table 6 (one or more of SEQ ID NOs: 75-77).

### Methods of Using the Mesothelin-Specific Binding Proteins and Method of Making and Using Cells Expressing the Mesothelin-Specific Binding Proteins

In a further aspect, this disclosure describes methods of using the mesothelin-specific binding proteins described herein. The mesothelin-specific binding protein (including, for example a TCR) as described herein may be used in any suitable application.

In one exemplary embodiment, the mesothelin-specific binding protein may be used to engineer a cell (also referred to herein as “target cell”) that overexpresses a mesothelin-specific binding protein. Exemplary cells include lymphocytes including, for example, T cell, NK (natural killer) cells, NKT cells (natural killer T cells); pluripotent cells including, for example, induced pluripotent stem cells (iPSCs); lymphocytes derived from pluripotent cells, etc. Combinations of target cells are also envisioned.

Thus, in various aspects, the disclosure provides a cell that overexpresses a mesothelin-specific binding protein. The disclosure also provides a composition comprising cells, including a composition comprising a mixed population of cells (e.g., T cells and NK cells), which overexpress the mesothelin-specific binding protein. In some embodiments, the cell that overexpresses a mesothelin-specific binding protein may preferably be a T cell or another cell that can express a T cell receptor. In some embodiments, the mesothelin-specific binding protein may preferably include a TCR. When the cell that overexpresses the mesothelin-specific binding protein is a T cell and the mesothelin-specific binding protein is a TCR, the resulting cell may be a TCR-engineered T cell (TCR-T).

In some embodiments, the mesothelin-specific binding protein is sufficiently avid to mediate lysing of a tumor in an antigen-specific manner (for example, via a TCR complexing with a peptide-MHC complex including mesothelin) but does not have sufficiently high affinity to result in off-tumor toxicity.

A cell that overexpresses a mesothelin-specific binding protein may be made ex vivo and then administered to a subject. In such embodiments, the cell may be expanded with specific antigen and/or various cytokines in vitro and then administered to the subject (Stromnes et al. Immunol Rev 257, 145-164 (2014)). Cells which overexpress a mesothelin-specific binding protein are produced by, e.g., exposing a cell to a construct (also referenced as an expression construct or vector) that expresses (encodes) a mesothelin-specific binding protein such that transduction occurs and mesothelin-specific binding protein is produced. As used herein, a construct that expresses a mesothelin-specific binding protein is one which comprises a nucleotide sequence encoding mesothelin-specific binding protein, such as any one or more of the nucleotide sequences set forth in Tables 1-6. Any suitable method of delivering a construct that encodes (i.e., expresses) a mesothelin-specific binding protein to a target cell of interest may be used. Examples of constructs include, but are not limited to, plasmids, viral vectors, non-episomal mammalian vectors and other expression vectors. Generally, a nucleic acid sequence encoding a desired polypeptide is operably linked to any number of regulatory elements (promoters, origin of replication, selectable markers, ribosomal binding sites, inducers, etc.). In various aspects, the polynucleotide encoding the mesothelin-specific binding protein (or fragment(s) thereof) are regulatable promoters, such as inducible promoters which upregulate transcription in the presence of a small molecule or other active agent. The constructs can be extra-chromosomal or integrating vectors. Constructs are described further below. Methods of culturing and expanding target cells, both before and after transduction, are known in the art.

Optionally, a polynucleotide encoding the mesothelin-specific binding protein is introduced into the TCR alpha constant (TRAC) locus within a host T cell genome. Targeted insertion of the polynucleotide into the TRAC locus may be accomplished using any of a variety of methods, such as those which employ recombinant adeno-associated virus and CRISPR/Cas9 systems. See, e.g., Rollins et. al., Nature Communications, In revision; Macleod et al. Molecular Therapy 25(4), 949-961 (2017); Eyquem et al. Nature 543, 113-7 (2017).

The disclosure further provides a composition comprising a population of cells that overexpress a mesothelin-specific binding protein described herein and a physiologically acceptable excipient or diluent. Optionally, the composition comprises a mixed population of cells (e.g., T cells and NK cells) that overexpress a mesothelin-specific binding protein described herein.

The disclosure further provides a method comprising administering a cell (or a composition comprising a population of cells) which overexpresses the mesothelin-specific binding protein described herein to a subject in need thereof. The subject may be any human or animal determined to benefit from the administration of the materials described herein. For example, in some embodiments, the subject is suspected of having or is suffering from a mesothelin-positive malignancy including, for example, pancreatic ductal adenocarcinoma, ovarian cancer, lung cancer, mesothelioma, breast cancer, acute myeloid leukemia, glioma, etc. In some embodiments, administration of the cell overexpressing the mesothelin-specific binding protein may result in preventing, slowing, and/or managing a mesothelin-positive malignancy. In this regard, the disclosure contemplates a method of treating a cancer (e.g., a mesothelin-positive cancer) in a subject in need thereof, the method comprising administering a composition comprising cell(s) overexpressing the mesothelin-specific binding protein described herein in a therapeutically effective amount. The disclosure also provides a cell that overexpresses a mesothelin-specific binding protein for use in treating cancer (e.g., a mesothelin-positive cancer) or use of the cell in the preparation of a medicament for treating cancer (e.g., a mesothelin-positive cancer) in a subject in need thereof. The disclosure contemplates adoptive cell therapy (ACT) employing the cell described herein, which may be used to treat a mesothelin-positive malignancy.

Optionally, the subject is subjected to a lymphodepletion regimen prior to administering the cell that overexpresses a mesothelin-specific binding protein. Lymphodepletion therapy is understood in the art and comprises, for example, administration of chemotherapeutic agents, such as cyclophosphamide, fludarabine, gemcitabine, abraxane, pentostatin, or bendamustine, or irradiation (e.g., total body irradiation).

Administration may be a single dose or multiple doses. The amount or dose of an active agent (i.e., the “effective amount”) administered is sufficient to achieve a desired biological effect, e.g., a therapeutic or prophylactic response, in the subject over a reasonable time frame. In some embodiments, the dose is an effective amount as determined by the standard methods.

In some embodiments, a cell expressing a mesothelin-specific binding protein may be administered in combination with another therapy. For example, administration may be combined with a strategy to target a suppressive tumor microenvironment, a strategy to target suppressive cells (including, for example, Tregs, myeloid-derived suppressor cells (MDSCs), tumor associated macrophages (TAMs), B cells, and cancer-associated fibroblasts (CAFs)), monocytes, and/or a strategy to target a factor of a suppressive tumor microenvironment (anti-TGFβ, anti-TGFBR2, anti-TGFBR1, FAK-inhibition, tyrosine kinase inhibitors, map kinase inhibitors, anti-IL-10, anti-CXCR4, etc). In another example, administration may be combined with an oncolytic viral therapy. In a further example, administration may be combined with a vaccination strategy that includes Trivax, Bivax, or a recombinant vaccine. Additional therapies suitable for use in connection with the methods described herein include, for example, another anti-tumor therapy.

Exemplary other therapies include administration of a cytokine (e.g., IL-2, IL-7, IL-15, and/or IL-21) or cytokine complexes (IL15/IL15RA), chemotherapy, radiotherapy, immunosuppressive therapy (including, for example, antibody therapy), surgery, etc. Common chemotherapeutics include, but are not limited to, abraxane, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, 5-fluorouracil, and the like.

Alternatively or in addition, the treatment regimen which comprises administration of the cell which overexpresses the mesothelin-specific binding protein described herein further comprises use of therapies which expand dendritic cells or change the tumor microenvironment (e.g., CD40 agonists or FLT3L), therapies that promote type I interferon (IFN) innate response (e.g., administration of adjuvants, including Poly:IC), therapies that promote TCR signaling and tumor cell recognition (such as “vaccination” or oncolytic virus strategies comprising administration of mesothelin peptide or administration of mRNA-LNP encoding mesothelin or mesothelin peptides alone), or administration of heterolytic peptides, such as altered peptide ligands. Any of the co-therapies described herein may be administered using any suitable route of administration, including intratumoral, systemic, or peritoneal administration.

In various aspects, the subject is administered a CD40 agonist. CD40 agonists are known in the art and include, e.g., CD40 ligand, selicrelumab, APX005M (Apexigen), ChiLob7/4, ADC-1013 (Janssen), SEA0CD40 (Seagen), CDX-1140 (Celldex). Further information regarding CD40 agonists is provided in, e.g., Vonderheide, Annual Review of Medicine, 71, 47-58 (2020).

In various aspects, the subject is administered a mesothelin peptide or an expression construct that expresses a mesothelin peptide, such as an mRNA vaccine or a virus-based vaccine engineered to express the mesothelin peptide. Exemplary mesothelin peptides include those comprising the amino acid sequence of SEQ ID NO: 1 or SEQ ID NO: 2. Alternative mesothelin peptides include the peptide of FIG. 10 or alternate fragments thereof.

In various aspects, the subject is administered an adjuvant that promotes a type I IFN innate response in the subject. Examples of adjuvants include, but are not limited to, polyinosinic:polycytidylic acid (Poly:IC), a STING (stimulator of interferon genes) peptide, and double stranded RNA that stimulates toll-like receptor 3 (TLR3). STING is described in, e.g., Barber, Nat Rev Immunol., 15(12), 760-770 (2015), incorporated herein by reference in its entirety.

Alternatively or in addition, the method further comprises administering an immune checkpoint inhibitor to the subject. An “immune checkpoint inhibitor” is any agent that that decreases, blocks, inhibits, abrogates or interferes with the function of a protein of an immune checkpoint pathway. Proteins of the immune checkpoint pathway regulate immune responses and, in some instances, prevent T cells from attacking cancer cells. In various aspects, the protein of the immune checkpoint pathway is, for example, CTLA-4, PD-1, PD-L1, PD-L2, B7-H3, B7-H4, TIGIT, VISTA, LAG3, CD112 TIM3, BTLA, or co-stimulatory receptor ICOS, OX40, 41BB, or GITR. In various aspects, the immune checkpoint inhibitor is a small molecule, an inhibitory nucleic acid, or an inhibitor polypeptide. In various aspects, the immune checkpoint inhibitor is an antibody, antigen-binding antibody fragment, or an antibody protein product, that binds to and inhibits the function of the protein of the immune checkpoint pathway (e.g., an antibody or fragment thereof that binds PD-1, PD-L1, CTLA4, Lag3, Tigit, Tim3, and the like). Suitable immune checkpoint inhibitors which are antibodies, antigen-binding antibody fragments, or an antibody protein products are known in the art and include, but are not limited to, ipilimumab (CTLA-4; Bristol Meyers Squibb), nivolumab (PD-1; Bristol Meyers Squibb), pembrolizumab (PD-1; Merck), atezolizumab (PD-L1; Genentech), avelumab (PD-L1; Merck), and durvalumab (PD-L1; Medimmune) (Wei et al., Cancer Discovery 8: 1069-1086 (2018)). Other examples of immune checkpoint inhibitors include, but are not limited to, IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); MGA271 (B7-H3: MacroGenics); and TSR-022 (TIM3; Tesaro).

In the context of combination therapies (i.e., regimens comprising administration of the cell overexpressing the mesothelin-specific binding protein and further administration of other active agents), the cell can be administered prior to, concurrent with, or after administration of one or more other active agents. The administration of the cell and other therapies need not occur simultaneously, although the disclosure contemplates embodiments wherein the components are included in the same pharmaceutical composition and administered together. The disclosure also provides a method wherein the cell and one or more other therapies (i.e., active agents) are present in separate pharmaceutical compositions which are administered in parallel or administered near in time. The cell and one or more other active agents may be administered serially (e.g., within minutes, hours, days, or weeks within each other), in any order.

In various aspects, the method of the disclosure comprises administering to a subject in need thereof (a) a composition comprising cells overexpressing a mesothelin-specific binding protein described herein and further comprises administering (b1) an agent which expands dendritic cells or changes the tumor microenvironment and/or (b2) an adjuvant and/or (b3) a cytokine and/or (b4) mesothelin or fragment thereof or a nucleic acid which encodes mesothelin or a fragment thereof. Optionally, (b1) the agent which expands dendritic cells or changes the tumor microenvironment is a CD40 agonist, such as an antibody which binds CD40 and increases CD40 activity. Optionally. (b2) the adjuvant is polyinosinic:polycytidylic acid (Poly:IC) or a double stranded RNA that stimulates toll-like receptor 3 (TLR3). Optionally, (b3) is interleukin-2. Optionally, the agents of (a) and (b1) and/or (b2) and/or (b3) and/or (b4) may be administered together or separately, in any order and within any time frame. For example, in an exemplary aspect, a dose of (a) cells is administered concurrently with (close in time, e.g., the same day) as (b2) the adjuvant. (b3) the cytokine (e.g., IL-2), and (b4) the mesothelin peptide or fragment thereof (or nucleic acid). A second dose of (a) cells is administered at a later timepoint, concurrent with (b2) the adjuvant, (b3) the cytokine. (b4) the mesothelin peptide or fragment thereof (or nucleic acid), and (b1) the agent which expands dendritic cells or changes the tumor microenvironment (e.g., CD40 agonist). A third dose of (a) cells may be administered with (b2), (b3), and (b4). This treatment regimen is provided to illustrate a representative aspect of the method disclosed herein. In various aspects, the method comprises (a) administering the composition comprising the cells described herein and (b) administering at a later timepoint (i) a mesothelin peptide or a construct encoding a mesothelin peptide and (ii) an adjuvant to the subject.

### Other Modifications to Cells

In some embodiments, a cell that overexpresses a mesothelin-specific TCR includes other modifications, such as modifications that affect (interfere with) a suppressive tumor environment. For example, a cell that overexpresses a mesothelin-specific binding protein may also overexpress a molecule that interferes with inhibitory receptor expression (for example, signaling by PD-1, Tim-3, CTLA-4, Lag-3. TIGIT, VISTA, TGFBR2, IL10, TGFBR1, TNFR1, etc.). Alternatively or in addition, the cell is optionally engineered to overexpress a molecule that interferes with suppressive cytokine signaling (for example, signaling mediated through specific cytokine receptors for IL-6, IL-10, TGFβ, IL-27, TNFα, or IFNβ). Alternatively or in addition, the cell is optionally engineered to overexpress a molecule that renders T cells resistant to a program of T cell exhaustion or promotes resident memory or both (for example, TOX, Hobit, Tcf7, Helios, Tbet, Klrg1, or CD103).

The cell may optionally be modified to be refractory to inhibitory receptor signaling (for example, signaling via PD-1, Tim-3, CTLA-4, LAG-3, TIGIT, VISTA, TGFβR2, IL10, TGFβR1, TNFR1, etc.). In this regard, the cell may be modified to reduce receptor expression (e.g., suppressive cytokine receptor(s)) or introduce mutations within receptors to disrupt signaling. Similarly, the cell may be modified to reduce expression of, or introduce mutations within, immune inhibitory proteins (e.g., PD1. LAG-3, TIM-3, TIGIT, SHP1, IL-10, CBLB, or DGKA). For example, the cell described herein may be further modified to reduce expression of transforming growth factor beta (TGFβ) receptor, such as TGFβ receptor 1 or TGFβ receptor 2 (TGFβR1 or TGFβR2). In an exemplary aspect of the disclosure, the cell overexpresses a mesothelin-specific binding protein (e.g., TCR) and is modified to reduce expression (i.e., knock out or knock down expression) of TGFβR2. The sequence of TGFβR2 is provided in FIG. 10. The cell also may be optionally engineered to be refractory to suppressive cytokine signallying (for example, signaling mediated through specific cytokine receptors for IL-6, IL-10, TGFβ, IL-27, TNFα, or IFNβ).

In another exemplary embodiment, a cell that overexpresses a mesothelin-specific binding protein may be modified to abrogate autocrine IL-10 production or TNFα production. Methods of knocking out or knocking down endogenous proteins in a host cell are known in the art and include, e.g., gene editing (using, e.g., zinc fingers nucleases (ZFNs), transcription activator-like effectors nucleases (TALENs), or CRISPR-Cas (clustered regularly interspaced short palindromic repeats-CRISPR associated) systems) or shRNA. Gene editing systems may modify the sequence of the target protein of interest or a regulatory element and/or non-coding region associated with the target gene. As merely an example of CRISPR systems, adenoviral delivery of the CRISPR/Cas9 system is described in Holkers et al., Nature Methods (2014), 11(10): 1051-1057, which is incorporated by reference in its entirety.

In a further exemplary embodiment, a cell that overexpresses a mesothelin-specific binding protein may be modified to express another chimeric costimulatory receptor (or combination thereof). Exemplary chimeric costimulatory receptors include, but are not limited to, NKG2A-NKG2D fusion proteins, CD8-41BB, CD8-MyD88-CD40, CD8-CD40, TGFBR2-41BB, TGFBR1-41BB, PD1-41BB, TIGIT-41BB, TIGIT-CD28, TIGIT-MYD88, CD40L, LAG3-41BB, and LAG3-CD28 costimulatory proteins, etc.

In a further exemplary embodiment, a cell that overexpresses a mesothelin-specific binding protein may be modified to express or overexpress an anti-tumor factor. Exemplary anti-tumor factors include, but are not limited to, dendritic cell attracting chemokines (for example, Flt3L, Xcl1, and IL-12), pro-inflammatory cytokines (for example, IL-2, IL-21, IL-15, IL-7, and IL-12), CD40 Ig, and CD40 ligand or peptides to initiate activation of endogenous virus- or tumor-specific T cells, or peptides of the mesothelin sequence itself.

### Administration of a Mesothelin-Specific Binding Protein to a Target Cell

In a further aspect, this disclosure describes a method that includes in vivo delivery of a mesothelin-specific binding protein or a construct that expresses a mesothelin-specific binding protein to a target cell of a subject. In this regard, a cell that overexpresses a mesothelin-specific binding protein may be produced in vivo by administering a mesothelin-specific binding protein or a construct that expresses a mesothelin-specific binding protein to a target cell of a subject.

Any suitable method of delivering a construct that expresses a mesothelin-specific binding protein to a target cell of interest may be used. Exemplary methods involve the use of viral vectors. Viral vectors may include any suitable viral vectors including, for example, retrovirus, adenovirus, parvovirus (for example, adeno-associated viruses), coronavirus, negative strand RNA viruses such as ortho-myxovirus (for example, influenza virus), rhabdovirus (for example, rabies and vesicular stomatitis virus), paramyxovirus (for example, measles and Sendai), positive strand RNA viruses such as picornavirus and alphavirus, and double-stranded DNA viruses including adenovirus, herpesvirus (for example, Herpes Simplex virus types 1 and 2, Epstein-Barr virus, cytomegalovirus), and poxvirus (for example, vaccinia, fowlpox, and canarypox). Other viruses that may be used as viral vectors include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus.

Exemplary target cells include tumor resident immune cells including, for example, T cells, NK cells, NKT cells, pluripotent cells (including, for example induced pluripotent stem cells (iPSCs)), and lymphocytes derived from pluripotent cells. Combinations of target cells are also envisioned.

### Administration, Compositions, and Kits

A composition including a cell that overexpresses a mesothelin-specific binding protein (or a mesothelin-specific binding protein itself, or a construct comprising a polynucleotide that encodes all or part of a mesothelin-specific binding protein) may be formulated in pharmaceutical preparations in a variety of forms adapted to the chosen route of administration. In some embodiments, the composition may include, for example, a pharmaceutically acceptable carrier, diluent, or excipient. One of skill will understand that the composition will vary depending on mode of administration and dosage unit. For example, for parenteral administration, isotonic saline may be used. Other suitable carriers include, but are not limited to alcohol, phosphate buffered saline, and other balanced salt solutions.

The composition (and optional co-therapies) may be administered in a variety of ways, including, but not limited to, intravenous, intraperitoneal, and intramuscular delivery. Other clinically acceptable methods include, but are not limited to, intralesional administration, intratumoral administration, and via an afferent lymph vessel. Bolus injection and continuous infusion are contemplated, as is localized administration, e.g., at a site of disease.

In some embodiments, the cell overexpressing the mesothelin-specific binding protein (or a mesothelin-specific binding protein itself, or a construct comprising a polynucleotide that encodes all or part of a mesothelin-specific binding protein) is provided in a kit. In exemplary aspects, the kit comprises the cell(s) (or protein or construct) as a unit dose (i.e., a discrete amount dispersed in a suitable carrier). In exemplary aspects, the kit comprises several unit doses, e.g., a week or month supply of unit doses, optionally, each of which is individually packaged or otherwise separated from other unit doses. In some embodiments, the components of the kit/unit dose are packaged with instructions for administration to a subject. In some embodiments, the kit comprises one or more devices for administration to a subject, e.g., a needle and delivery device (such as a syringe), and the like. In some aspects, the antigen-binding protein is pre-packaged in a ready to use form, e.g., a syringe, an intravenous bag, etc. In some aspects, the kit further comprises other therapeutic or diagnostic agents or pharmaceutically acceptable carriers (e.g., solvents, buffers, diluents, etc.), including any of those described herein.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

### Exemplary Mesothelin-Specific Binding Protein (MSLN₂₀₋₂₈) Aspects

A1. A mesothelin-specific binding protein comprising:
   a TCRα CDR3 having a peptide sequence of CAASGNTDKLIF (SEQ ID NO:3);
   a TCRα CDR3 having a peptide sequence of CAFYMDSNYQLIW (SEQ ID NO:4); or
   a TCRα CDR3 having a peptide sequence of CAVIPNNNARLMF (SEQ ID NO:5); and/or
   a TCRβ CDR3 having a peptide sequence of CASRPGWSYEQYF (SEQ ID NO:6);
   a TCRβ CDR3 having a peptide sequence of CASSEWTAEQYF (SEQ ID NO:7); or
   a TCRβ CDR3 having a peptide sequence of CASGQGTEAFF (SEQ ID NO:8).
A2. A mesothelin-specific binding protein comprising:
   a T cell receptor (TCR) α-chain variable (V_{α}) domain comprising
      a TCRα CDR3 having a peptide sequence of CAASGNTDKLIF (SEQ ID NO:3); a TCRα CDR3 having a peptide sequence of CAFYMDSNYQLIW (SEQ ID NO:4); or a TCRα CDR3 having a peptide sequence of CAVIPNNNARLMF (SEQ ID NO:5); and
   a TCR B-chain variable (V_{β}) domain.
A3. A mesothelin-specific binding protein comprising:
   a T cell receptor (TCR) β-chain variable (V_{β}) domain comprising
      a TCRβ CDR3 having a peptide sequence of CASRPGWSYEQYF (SEQ ID NO:6); a TCRβ CDR3 having a peptide sequence of CASSEWTAEQYF (SEQ ID NO:7); or a TCRβ CDR3 having a peptide sequence of CASGQGTEAFF (SEQ ID NO:8); and
   a TCR α-chain variable (V_{α}) domain.
A4. A mesothelin-specific binding protein comprising:
   a T cell receptor (TCR) α-chain variable (V_{α}) domain comprising
      a TCRα CDR3 having a peptide sequence of CAASGNTDKLIF (SEQ ID NO:3); a TCRα CDR3 having a peptide sequence of CAFYMDSNYQLIW (SEQ ID NO:4); or a TCRα CDR3 having a peptide sequence of CAVIPNNNARLMF (SEQ ID NO:5); and
   a TCR B-chain variable (V_{β}) domain comprising
      a TCRβ CDR3 having a peptide sequence of CASRPGWSYEQYF (SEQ ID NO:6); a TCRβ CDR3 having a peptide sequence of CASSEWTAEQYF (SEQ ID NO:7); or a TCRβ CDR3 having a peptide sequence of CASGQGTEAFF (SEQ ID NO:8).
A5. The mesothelin-specific binding protein of any of Aspects A1 to A4, comprising:
   a TCRα CDR3 having a peptide sequence of CAASGNTDKLIF (SEQ ID NO:3) and a TCRβ CDR3 having a peptide sequence of CASRPGWSYEQYF (SEQ ID NO:6);
   a TCRα CDR3 having a peptide sequence of CAFYMDSNYQLIW (SEQ ID NO:4) and a TCRβ CDR3 having a peptide sequence of CASSEWTAEQYF (SEQ ID NO:7); or
   a TCRα CDR3 having a peptide sequence of CAVIPNNNARLMF (SEQ ID NO:5) and a TCRβ CDR3 having a peptide sequence of CASGQGTEAFF (SEQ ID NO:8).
A6. The mesothelin-specific binding protein of any of Aspects A1 to A5, comprising:
   the peptide sequence of Vα TRAV29/DV5*01 and TRAJ34*01;
   the peptide sequence of Vα TRAV24*01 and TRAJ33*01; or
   the peptide sequence of Vα TRAV8-6*022 and TRAJ31*01.
A7. The mesothelin-specific binding protein of any of Aspects A1 to A6, comprising
   the peptide sequence of Vβ TRBV2*01; TRBJ2-7*01 and TRBD1*01;
   the peptide sequence of Vβ TRBV6-1*01; TRBJ2-3*01; and TRBD1*01; or
   the peptide sequence of Vβ TRBV4-2*01; TRBJ1-1*01; and TRBD1*01.
A8. The mesothelin-specific binding protein of any of Aspects A1 to A7, comprising the Valpha peptide sequence on Table 1, Table 2, or Table 3.
A9. The mesothelin-specific binding protein of any of Aspects A1 to A8, comprising a Vbeta peptide sequence of Table 1, Table 2, or Table 3.
A10. The mesothelin-specific binding protein of any of Aspects A1 to A9, comprising a Valpha-Valpha constant peptide sequence of Table 1, Table 2, or Table 3.
A11. The mesothelin-specific binding protein of any of Aspects A1 to A10, comprising a Vbeta-Vbeta constant peptide sequence of Table 1, Table 2, or Table 3.
A12. The mesothelin-specific binding protein of any of Aspects A1 to A11, comprising a Vbeta-Vbeta constant and a Valpha-Valpha constant peptide sequence of Table 1, Table 2, or Table 3.
A13. The mesothelin-specific binding protein of any of Aspects A1 to A12, comprising a Vbeta-Vbeta constant-P2A-Valpha-Valpha constant peptide sequence of Table 1, Table 2, or Table 3.
A14. The mesothelin-specific binding protein of any of Aspects A1 to A5, wherein the mesothelin-specific binding protein comprises a V_{α} domain that is at least about 90% identical to a Valpha-Valpha peptide sequence of Table 1, Table 2, or Table 3, and comprises a Vβ domain that is at least about 90% identical to a Vbeta-Vbeta peptide sequence of Table 1, Table 2, or Table 3.
A15. The mesothelin-specific binding protein of Aspect A14,
   wherein none of the CDRs of the mesothelin-specific binding protein include a mutation relative to the CDRs of the Vbeta-Vbeta constant peptide sequence of Table 1, Table 2, or Table 3, or
   wherein TCRβ CDR3 does not include a mutation relative to the corresponding TCRβ CDR3 of the Vbeta-Vbeta constant peptide sequence of Table 1, Table 2, or Table 3.
A16. The mesothelin-specific binding protein of Aspect A14 or A15,
   wherein none of the CDRs of the mesothelin-specific binding protein include a mutation relative to the CDRs of the Valpha-Valpha constant peptide sequence of Table 1, Table 2, or Table 3, or
   wherein TCRα CDR3 does not include a mutation relative to the corresponding TCRα CDR3 of the Valpha-Valpha constant peptide sequence of Table 1, Table 2, or Table 3.
A17. The mesothelin-specific binding protein of any of Aspects A1 to A16, wherein the mesothelin-specific binding protein binds to a tetramer comprising amino acids 20-28 of human mesothelin with a K_{d} of less than 10⁻⁸ M, less than 10⁻⁹ M, less than 10⁻¹⁰ M, less than 10⁻¹¹ M, less than 10⁻¹² M, or less than 10⁻¹³ M.
A18. The mesothelin-specific binding protein of any of Aspects A1 to A17, comprising a TCR constant region.
A19. The mesothelin-specific binding protein of A18, wherein the mesothelin-specific binding protein comprises an TCRα constant region comprises a cysteine modification to induce preferential pairing of TCR constant regions.
A20. An isolated polynucleotide molecule encoding the mesothelin-specific binding protein of any of Aspects A1 to A19.
A21. The isolated polynucleotide molecule of Aspect A20, wherein the isolated polynucleotide molecule comprises a codon optimized sequence.
A21. A cell that overexpresses a mesothelin-specific binding protein of any of Aspects A1 to A19.
A22. The cell of Aspect A21, wherein the cell further overexpresses:
   a molecule that interferes with inhibitory receptor expression;
   a molecule that interferes with suppressive cytokine signaling;
   a molecule that renders T cells resistant to a program of T cell exhaustion and/or promotes resident memory;
   a chimeric costimulatory receptor; and/or
   an anti-tumor factor.
A23. The cell of Aspect A21 or A22, wherein the cell abrogates autocrine IL-10 production.

### Exemplary Mesothelin-Specific Binding Protein (MSLN₅₃₀₋₅₃₈) Aspects

B1. A mesothelin-specific binding protein comprising:
   a TCRα CDR3 having a peptide sequence of CAYLGTGTYKYIF (SEQ ID NO:9);
   a TCRα CDR3 having a peptide sequence of CAGGMESGGGADGLTF (SEQ ID NO:10); or
   a TCRα CDR3 having a peptide sequence of CALDTGFQKLVF (SEQ ID NO:11); and/or
   a TCRβ CDR3 having a peptide sequence of CASSSGGLGYTF (SEQ ID NO:12);
   a TCRβ CDR3 having a peptide sequence of CASTSTGGLKNTEAFF (SEQ ID NO:13); or
   a TCRβ CDR3 having a peptide sequence of CASSSLGDRNTEAFF (SEQ ID NO:14).
B2. A mesothelin-specific binding protein comprising:
   a T cell receptor (TCR) α-chain variable (V_{α}) domain comprising
      a TCRα CDR3 having a peptide sequence of CAYLGTGTYKYIF (SEQ ID NO:9); a TCRα CDR3 having a peptide sequence of CAGGMESGGGADGLTF (SEQ ID NO:10); or a TCRα CDR3 having a peptide sequence of CALDTGFQKLVF (SEQ ID NO:11); and
   a TCR β-chain variable (V_{β}) domain.
B3. A mesothelin-specific binding protein comprising:
   a T cell receptor (TCR) β-chain variable (V_{β}) domain comprising
      a TCRβ CDR3 having a peptide sequence of CASSSGGLGYTF (SEQ ID NO:12); a TCRβ CDR3 having a peptide sequence of CASTSTGGLKNTEAFF (SEQ ID NO:13); or a TCRβ CDR3 having a peptide sequence of CASSSLGDRNTEAFF (SEQ ID NO:14); and
   a TCR α-chain variable (V_{α}) domain.
B4. A mesothelin-specific binding protein comprising:
   a T cell receptor (TCR) α-chain variable (V_{α}) domain comprising
      a TCRα CDR3 having a peptide sequence of CAYLGTGTYKYIF (SEQ ID NO:9); a TCRα CDR3 having a peptide sequence of CAGGMESGGGADGLTF (SEQ ID NO:10); or a TCRα CDR3 having a peptide sequence of CALDTGFQKLVF (SEQ ID NO:11); and
   a TCR β-chain variable (V_{β}) domain comprising
      a TCRβ CDR3 having a peptide sequence of CASSSGGLGYTF (SEQ ID NO:12); a TCRβ CDR3 having a peptide sequence of CASTSTGGLKNTEAFF (SEQ ID NO:13); or a TCRβ CDR3 having a peptide sequence of CASSSLGDRNTEAFF (SEQ ID NO:14).
B5. The mesothelin-specific binding protein of any of Aspects B1 to B4, comprising:
   a TCRα CDR3 having a peptide sequence of CAYLGTGTYKYIF (SEQ ID NO:9) and a TCRβ CDR3 having a peptide sequence of CASSSGGLGYTF (SEQ ID NO:12);
   a TCRα CDR3 having a peptide sequence of CAGGMESGGGADGLTF (SEQ ID NO:10) and a TCRβ CDR3 having a peptide sequence of CASTSTGGLKNTEAFF (SEQ ID NO:13); or
   a TCRα CDR3 having a peptide sequence of CALDTGFQKLVF (SEQ ID NO:11) and a TCRβ CDR3 having a peptide sequence of CASSSLGDRNTEAFF (SEQ ID NO:14).
B6. The mesothelin-specific binding protein of any of Aspects B1 to B5, comprising:
   the peptide sequence of Vα TRAV38-1*04 and TRAJ40*01;
   the peptide sequence of Vα TRAV38-2/DV8*01 and TRAJ40*01;
   the peptide sequence of Vα TRAV27*03 and TRAJ45*01; or
   the peptide sequence of Vα TRAV9-2*01 and TRAJ8*01.
B7. The mesothelin-specific binding protein of any of Aspects B1 to B6, comprising
   the peptide sequence of Vβ TRBV27*01 and TRBJ2-6*01;
   the peptide sequence of Vβ TRBV7-9*01; TRBJ1-1*01 and TRBD1*01; or
   the peptide sequence of Vβ TRBV27*01; TRBJ1-1*01; and TRBD1*01.
B8. The mesothelin-specific binding protein of any of Aspects B1 to B7, comprising the Valpha peptide sequence on Table 4, Table 5, or Table 6.
B9. The mesothelin-specific binding protein of any of Aspects B1 to B8, comprising a Vbeta peptide sequence of Table 4, Table 5, or Table 6.
B10. The mesothelin-specific binding protein of any of Aspects B1 to B9, comprising a Valpha-Valpha constant peptide sequence of Table 4, Table 5, or Table 6.
B11. The mesothelin-specific binding protein of any of Aspects B1 to B10, comprising a Vbeta-Vbeta constant peptide sequence of Table 4, Table 5, or Table 6.
B12. The mesothelin-specific binding protein of any of Aspects B1 to B11, comprising a Vbeta-Vbeta constant and a Valpha-Valpha constant peptide sequence of Table 4, Table 5, or Table 6.
B13. The mesothelin-specific binding protein of any of Aspects B1 to B12, comprising a Vbeta-Vbeta constant-P2A-Valpha-Valpha constant peptide sequence of Table 4, Table 5, or Table 6.
B14. The mesothelin-specific binding protein of any of Aspects B1 to B5, wherein the mesothelin-specific binding protein comprises a V_{α} domain that is at least about 90% identical to a Valpha-Valpha peptide sequence of Table 4, Table 5, or Table 6, and comprises a Vβ domain that is at least about 90% identical to a Vbeta-Vbeta peptide sequence of Table 4, Table 5, or Table 6.
B15. The mesothelin-specific binding protein of Aspect B14,
   wherein none of the CDRs of the mesothelin-specific binding protein include a mutation relative to the CDRs of the Vbeta-Vbeta constant peptide sequence of Table 4, Table 5, or Table 6, or
   wherein TCRβ CDR3 does not include a mutation relative to the corresponding TCRβ CDR3 of the Vbeta-Vbeta constant peptide sequence of Table 4, Table 5, or Table 6.
B16. The mesothelin-specific binding protein of Aspect B14 or B15,
   wherein none of the CDRs of the mesothelin-specific binding protein include a mutation relative to the CDRs of the Valpha-Valpha constant peptide sequence of Table 4, Table 5, or Table 6, or
   wherein TCRα CDR3 does not include a mutation relative to the corresponding TCRα CDR3 of the Valpha-Valpha constant peptide sequence of Table 4, Table 5, or Table 6.
B17. The mesothelin-specific binding protein of any of Aspects B1 to B16, wherein the mesothelin-specific binding protein binds to a tetramer comprising amino acids 20-28 of human mesothelin with a K_{d} of less than 10⁻⁸ M, less than 10⁻⁹ M, less than 10⁻¹⁰ M, less than 10⁻¹¹ M, less than 10⁻¹² M, or less than 10⁻¹³ M.
B18. The mesothelin-specific binding protein of any of Aspects B1 to B17, comprising a TCR constant region.
B19. The mesothelin-specific binding protein of B18, wherein the mesothelin-specific binding protein comprises an TCRα constant region comprises a cysteine modification to induce preferential pairing of TCR constant regions.
B20. An isolated polynucleotide molecule encoding the mesothelin-specific binding protein of any of Aspects B1 to B19.
B21. The isolated polynucleotide molecule of Aspect B20, wherein the isolated polynucleotide molecule comprises a codon optimized sequence.
B22. A cell that overexpresses a mesothelin-specific binding protein of any of Aspects B1 to B19.
B23. The cell of Aspect B22, wherein the cell overexpresses:
   a molecule that interferes with inhibitory receptor expression;
   a molecule that interferes with suppressive cytokine signaling;
   a molecule that renders T cells resistant to a program of T cell exhaustion and/or promotes resident memory;
   a chimeric costimulatory receptor; and/or
   an anti-tumor factor.
B24. The cell of Aspect B22 or B23, wherein the cell abrogates autocrine IL-10 production.

### Methods of Using

C1. A method comprising delivery of the mesothelin-specific binding protein of any of Aspects A1 to A19 or B1 to B19 or a construct that expresses a mesothelin-specific binding protein of any of Aspects A1 to A19 or Aspects B1 to B19 to a target cell to produce a cell that overexpresses the mesothelin-specific binding protein.
C2. The method of Aspect C1, wherein the delivery of the mesothelin-specific binding protein comprises in vivo delivery to the target cell.
C3. The method of Aspect C2, wherein the target cell comprises a tumor resident immune cell.
C4. The method of Aspect C1, wherein the delivery of the mesothelin-specific binding protein comprises ex vivo delivery to the target cell.
C5. The method of any one of Aspects C2 to C4, wherein the mesothelin-specific binding protein or the construct that expresses a mesothelin-specific binding protein is delivered to the target cell via a viral vector.
C6. The method of any one of Aspects C1 to C5, wherein the target cell is a T cell, an NK cell, an NKT cell, a pluripotent cell, or a lymphocyte derived from a pluripotent cell, or a combination thereof.
C7. A method comprising
   administering the target cell of any one of Aspects C1 or C4 to C6 to a subject,
   administering a cell that overexpresses the mesothelin-specific binding protein of any of Aspects A1 to A19 or Aspects B1 to B19 to a subject.
C6. The method of any of Aspects C1 to C5, wherein the cell that overexpresses the mesothelin-specific binding protein and overexpresses:
   a molecule that interferes with inhibitory receptor expression;
   a molecule that interferes with suppressive cytokine signaling;
   a molecule that renders T cells resistant to a program of T cell exhaustion and/or promotes resident memory;
   a chimeric costimulatory receptor; and/or
   an anti-tumor factor.
C7. The method of any of Aspects C1 to C5, wherein the cell that overexpresses the mesothelin-specific binding protein abrogates autocrine IL-10 production.
C8. The method of any of Aspects C1 to C7, wherein the subject has or is suspected of having a mesothelin-positive malignancy.
C9. The method of any of Aspects C1 to C8, wherein the subject is a human.

The present invention is illustrated by the following examples. It is to be understood that the particular examples, materials, amounts, and procedures are to be interpreted broadly in accordance with the scope and spirit of the invention as set forth herein.

### Examples

All reagents, starting materials, and solvents used in the following examples were purchased from commercial suppliers (such as Sigma Aldrich, St. Louis, MO) and were used without further purification unless otherwise indicated.

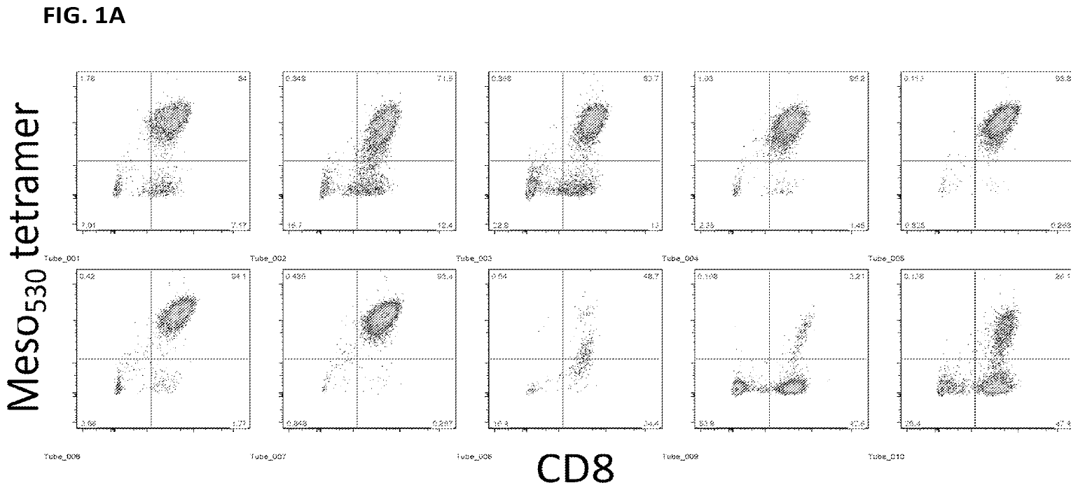

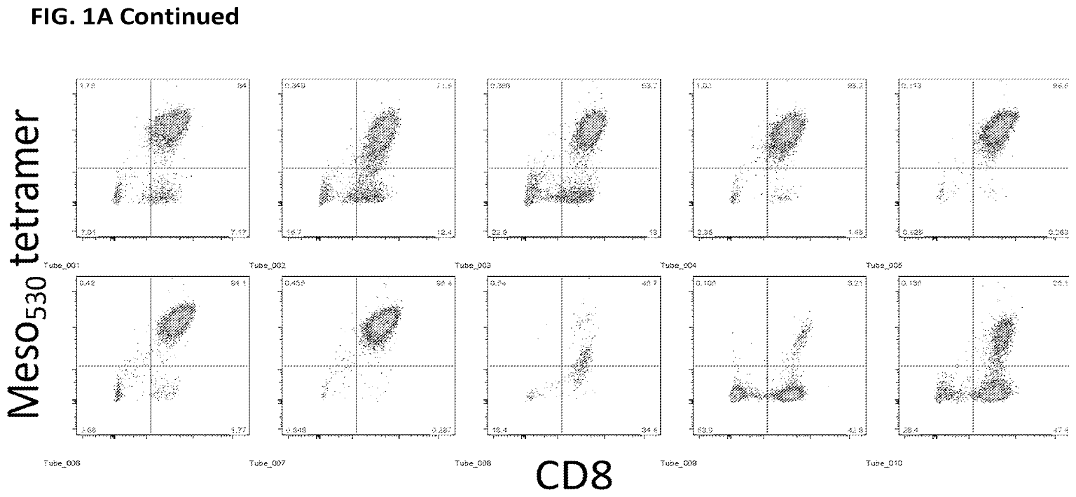

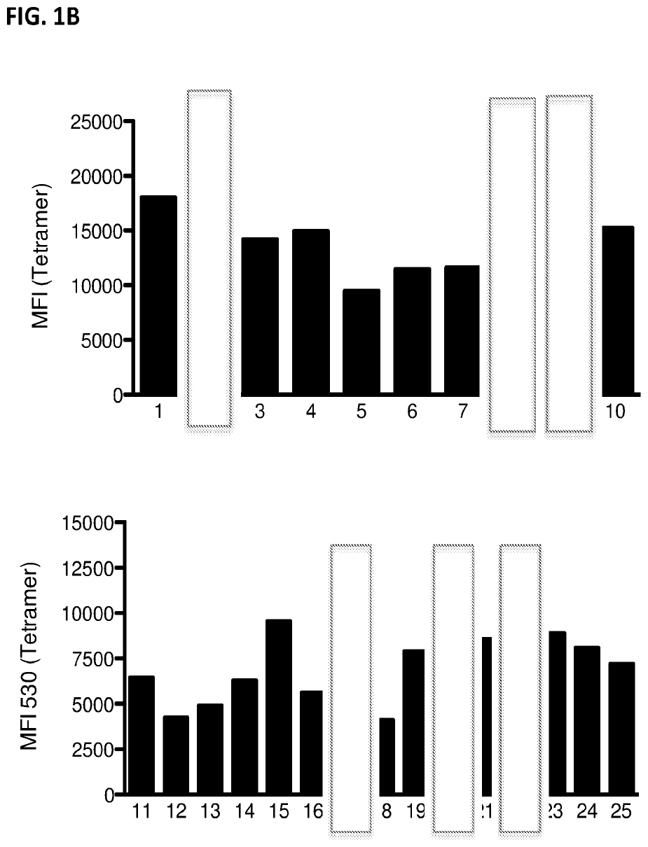

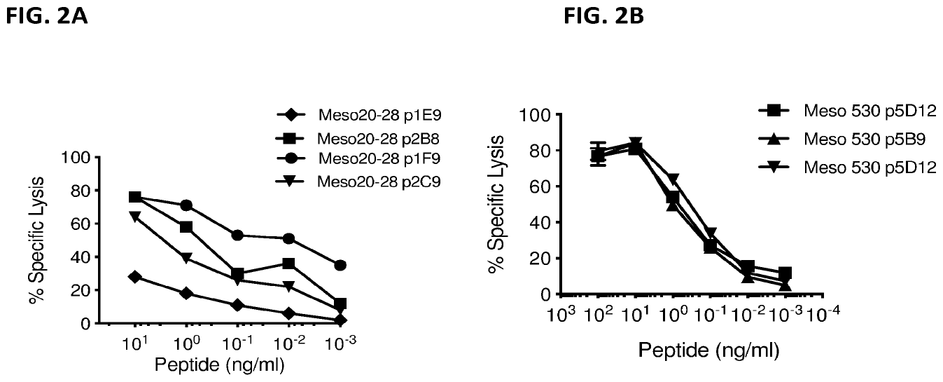

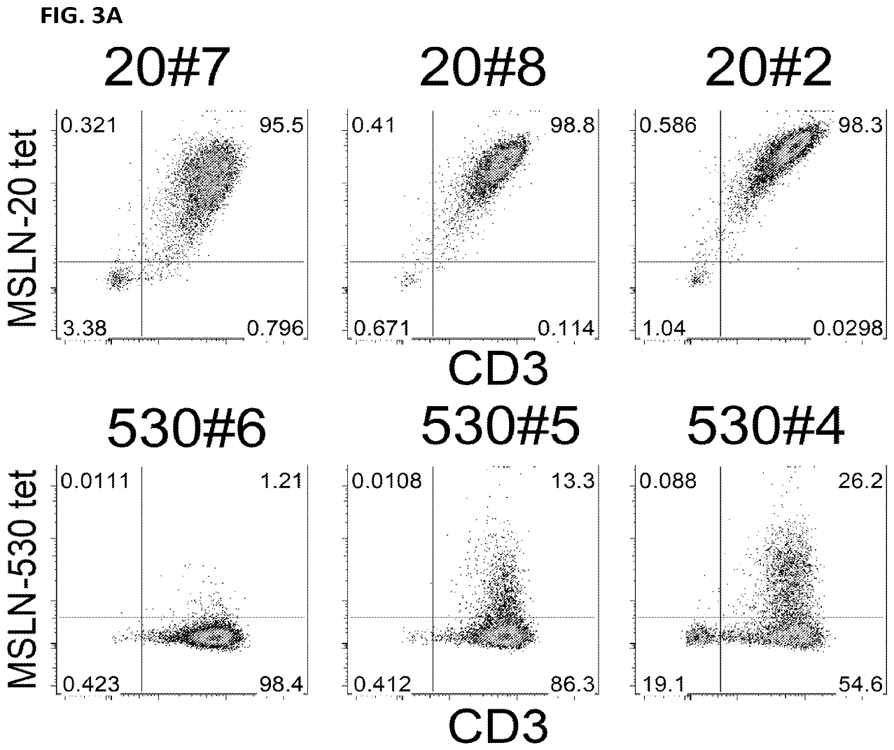

Example 1 describes generation of T cells reactive to many different MSLN epitopes. T cells reactive to MSLN₂₀₋₂₈ (SLLFLLFSL (SEQ ID NO:1)) or MSLN₅₃₀₋₅₃₈ (VLPLTVAEV (SEQ ID NO:2) were expanded (Stromnes et al. Cancer Cell 28, 638-652 (2015)). Human T cell lines were created and were screened for tetramer staining and validation of functional activity (FIG. 1, FIG. 2). As described in Example 2, following tetramer staining and validation of functional activity, numerous TCRs were cloned from the human T cell clones. These mesothelin-specific TCRs were recreated using gene blocks, codon optimized and cysteine modified (in the constant region to induce preferential pairing of the donor alpha and beta chain), and cloned into lentiviral vectors (Stromnes et al. Cancer Cell 28, 638-652 (2015), Rollins et al. Curr Protoc Immunol 129, e97 (2020)). The sequences of these codon-optimized and cysteine-modified mesothelin-specific TCRs as well as the CDR3 sequences of the Vα and Vβ domains are provided in Example 2 and Tables 1-6. Example 3 describes characterization of cells expressing the mesothelin-specific TCRs.

### Example 1—Screening of Human T Cell Lines

An attempt was made to generate T cells reactive to many different MSLN epitopes, but only T cells reactive to MSLN₂₀₋₂₈ (SLLFLLFSL (SEQ ID NO:1)) or MSLN₅₃₀₋₅₃₈ (VLPLTVAEV (SEQ ID NO:2)) expanded, as previously described (Stromnes et al. Cancer Cell 28, 638-652 (2015)).

Ten independent human T cell lines reactive to MSLN epitopes were screened for tetramer binding by flow cytometry. Results are shown in FIG. 1A-FIG. 1B.

MSLN-reactive TCRs were cloned from the human T cell lines which bound particularly well to tetramer (indicated by the boxes in FIG. 1B).

Human T cell lines reactive to MSLN epitopes were also screened for specific lysis of T2 cells pulsed with titrating concentrations of MSLN peptide. Results are shown in FIG. 2A-FIG. 2B.

### Example 2—Cloning of T Cell Receptors (TCRs)

Following tetramer staining and validation of functional activity (as described in Example 1), numerous TCRs were cloned from the human T cell clones.

Three TCRs reactive to MSLN₂₀₋₂₈ and three TCRs reactive to MSLN₅₃₀₋₅₃₈ were recreated using gene blocks, codon optimized and cysteine modified, and cloned into lentiviral vectors. (Stromnes et al. Cancer Cell 28, 638-652 (2015), Rollins et al. Curr Protoc Immunol 129, e97 (2020)). The cysteine modification is in the constant region, and induces preferential pairing of the exogenous TCR chains, thereby preventing mispairing with endogenous TCR chains.

Sequences of these codon-optimized and cysteine modified TCRs are shown in Tables 1-6.

### MSLN 20-28 Clone 2

MSLN₂₀₋₂₈ clone 2 is a TCR reactive to amino acids 20-28 of human mesothelin in the context of HLA-A201. Its TCRα includes TRAV29/DV5*01 TRAJ34*01. Its TCRβ includes TRBV2*01; TRBJ2-7*01; TRBD1*01.

Polynucleotide sequences of CDR3 TCRα, CDR3 TCRβ, codon optimized α variable region (CO-Valpha), codon optimized α constant region (CO-Valpha constant), codon optimized β variable region (CO-Vbeta), β constant region (CO-Vbeta constant), and the codon optimized α and β variable and constant regions, connected with a self-cleaving peptide (P2A) (CO-Vbeta-Vbeta constant-P2A-Valpha-Valpha constant), are shown in Table 1. In the Tables, underlined sequences correspond to CDR3 amino acid sequences, bolded correspond to Valpha or Vbeta sequences, and underlined and italics text denotes P2A sequences.

Peptide sequences of the CDR3 TCRα, CDR3 TCRβ, α variable region (Valpha), a constant region (Valpha constant), β variable region (Vbeta), β constant region (Vbeta constant), and a construct including Vbeta-Vbeta constant and Valpha-Valpha constant connected with a P2A sequence are also shown in Table 1.

### MSLN₂₀₋₂₈ Clone 7

MSLN₂₀₋₂₈ clone 7 is a TCR reactive to amino acids 20-28 of human mesothelin in the context of HLA-A201. Its TCRα includes TRAV24*01; TRAJ33*01. Its TCRβ includes TRBV6-1*01; TRBJ2-3*01; TRBD1*01.

Polynucleotide sequences of CDR3 TCRα, CDR3 TCRβ, and the codon optimized α and β variable and constant regions, connected with a self-cleaving peptide (P2A), are shown in Table 2.

Peptide sequences of the CDR3 TCRα, CDR3 TCRβ, a variable region (Valpha), α constant region (Valpha constant), β variable region (Vbeta), β constant region (Vbeta constant), and a construct including Vbeta-Vbeta constant and Valpha-Valpha constant connected with a P2A sequence are also shown in Table 2.

### MSLN₂₀₋₂₈ Clone 8

MSLN₂₀₋₂₈ clone 8 is a TCR reactive to amino acids 20-28 of human mesothelin in the context of HLA-A201. Its TCRα includes TRAV8-6*022; TRAJ31*01. Its TCRβ includes TRBV4-2*01; TRBJ1-1*01; TRBD1*01.

Polynucleotide sequences of CDR3 TCRα, CDR3 TCRβ, and the codon optimized α and β variable and constant regions, connected with a self-cleaving peptide (P2A), are shown in Table 3.

Peptide sequences of the CDR3 TCRα, CDR3 TCRβ, a variable region (Valpha), α constant region (Valpha constant), β variable region (Vbeta), β constant region (Vbeta constant), and a construct including Vbeta-Vbeta constant and Valpha-Valpha constant connected with a P2A sequence are also shown in Table 3.

### MSLN₅₃₀₋₅₃₈ Clone 4

MSLN₅₃₀₋₅₃₈ clone 4 is reactive to amino acids 530-538 of human mesothelin in the context of HLA-A201. Its TCRα includes TRAV38-1*04 (or TRAV38-2/DV8*01); TRAJ40*01. Its TCRβ includes TRBV27*01; TRBJ2-6*01 (no D region was identified using IMGT, available online at www.imgt.org).

Polynucleotide sequences of CDR3 TCRα, CDR3 TCRβ, codon optimized α variable region (CO-Valpha), codon optimized α constant region (CO-Valpha constant), codon optimized β variable region (CO-Vbeta), β constant region (CO-Vbeta constant), and the codon optimized α and β variable and constant regions, connected with a self-cleaving peptide (P2A) (CO-Vbeta-Vbeta constant-P2A-Valpha-Valpha constant), are shown in Table 4.

Peptide sequences of the CDR3 TCRα, CDR3 TCRβ, a variable region (Valpha), α constant region (Valpha constant), β variable region (Vbeta), β constant region (Vbeta constant), and a construct including Vbeta-Vbeta constant and Valpha-Valpha constant connected with a P2A sequence are also shown in Table 4.

### MSLN₅₃₀₋₅₃₈ Clone 5

MSLN₅₃₀₋₅₃₈ clone 5 is reactive to amino acids 530-538 of human mesothelin in the context of HLA-A201. Its TCRα includes TRAV27*03; TRAJ45*01. Its TCRβ includes TRBV7-9*01; TRBJ1-1*01; TRBD1*01.

Polynucleotide sequences of CDR3 TCRα, CDR3 TCRβ, and the codon optimized α and β variable and constant regions, connected with a self-cleaving peptide (P2A), are shown in Table 5.

Peptide sequences of the CDR3 TCRα, CDR3 TCRβ, α variable region (Valpha), a constant region (Valpha constant), β variable region (Vbeta), β constant region (Vbeta constant), and a construct including Vbeta-Vbeta constant and Valpha-Valpha constant connected with a P2A sequence are also shown in Table 5.

### MSLN₅₃₀₋₅₃₈ Clone 6

MSLN₅₃₀₋₅₃₈ clone 6 is reactive to amino acids 530-538 of human mesothelin in the context of HLA-A201. Its TCRα includes TRAV9-2*01; TRAJ8*01. Its TCRβ includes TRBV27*01; TRBJ1-1*01; TRBD1*01

Polynucleotide sequences of CDR3 TCRα, CDR3 TCRβ, and the codon optimized α and β variable and constant regions, connected with a self-cleaving peptide (P2A), are shown in Table 6.

Peptide sequences of the CDR3 TCRα, CDR3 TCRβ, a variable region (Valpha), a constant region (Valpha constant), β variable region (Vbeta), β constant region (Vbeta constant), and a construct including Vbeta-Vbeta constant and Valpha-Valpha constant connected with a P2A sequence are also shown in Table 6.

### Example 3—Characterization of T Cells Expressing the Cloned TCRs

The TCRs of Example 2 were expressed in both Jurkat T cell lines and primary human T cells as previously described (Stromnes et al. Cancer Cell 28, 638-652 (2015), Rollins et al. Curr Protoc Immunol 129, e97 (2020)).

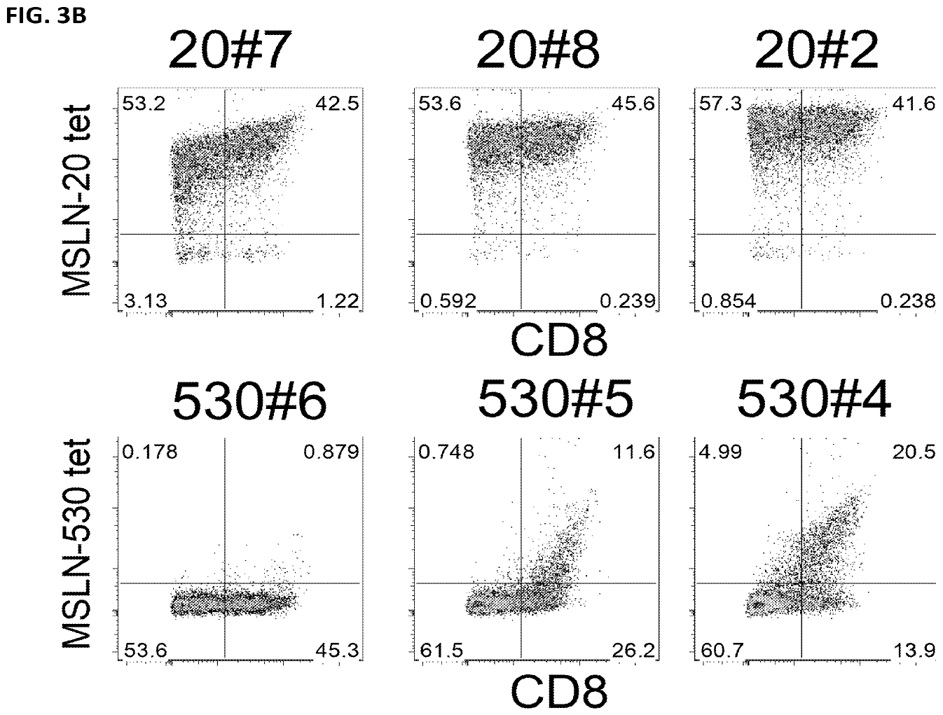

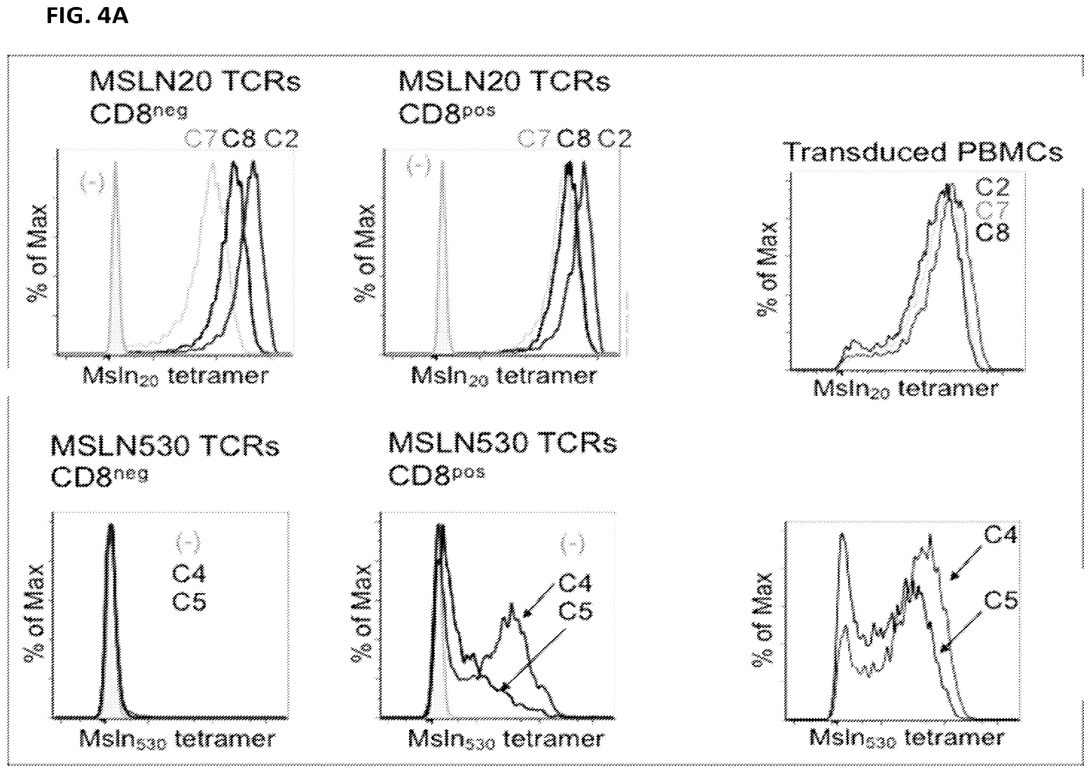

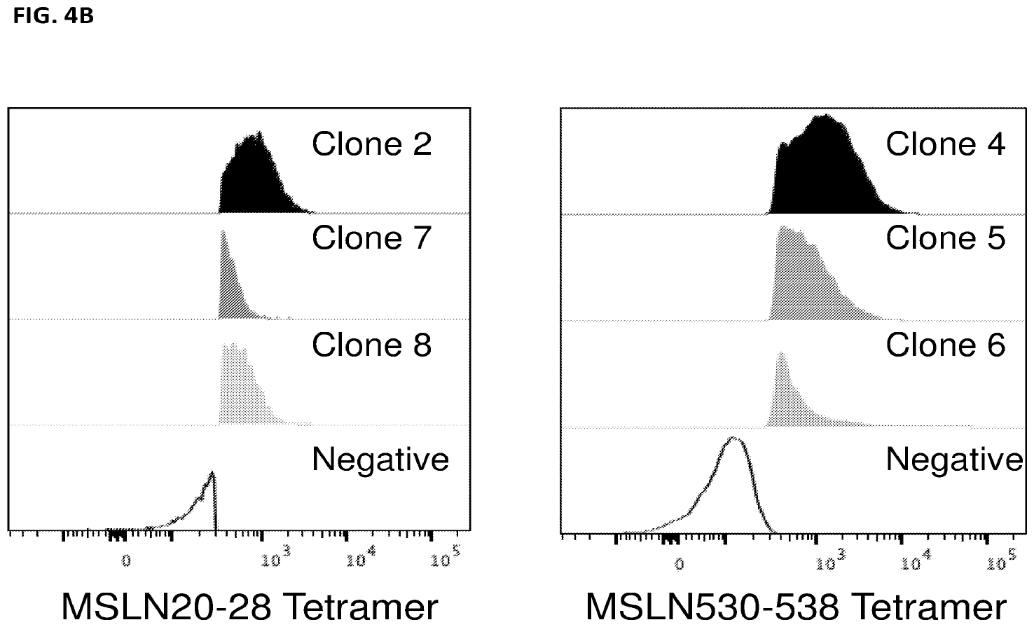

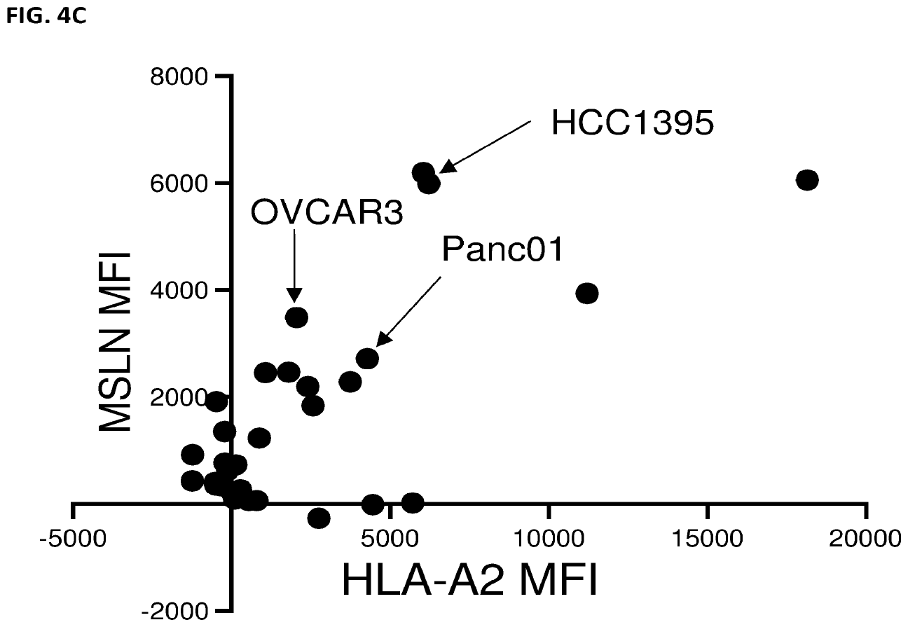

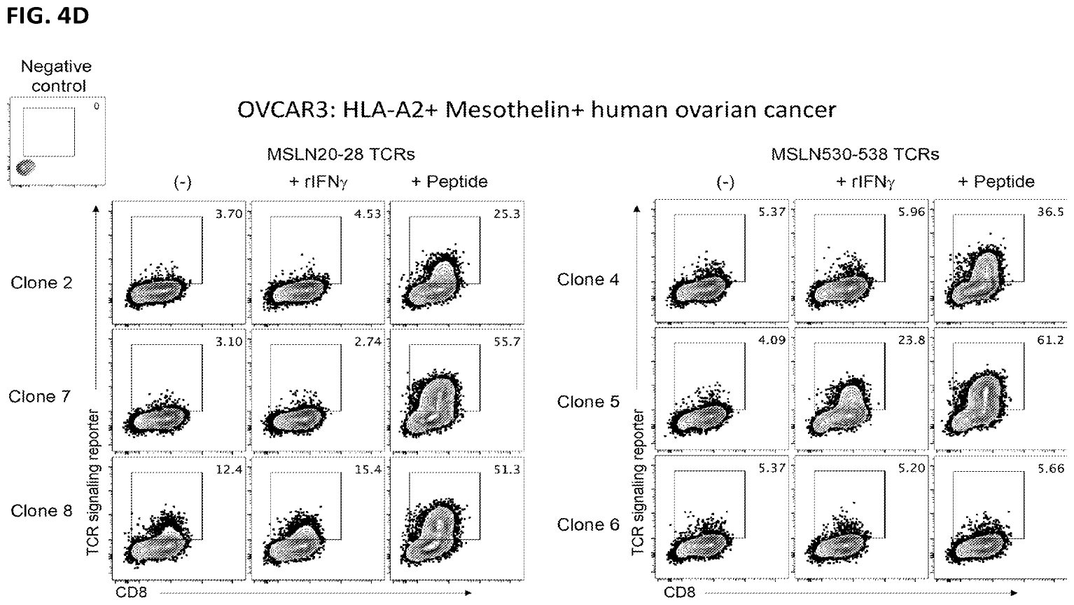

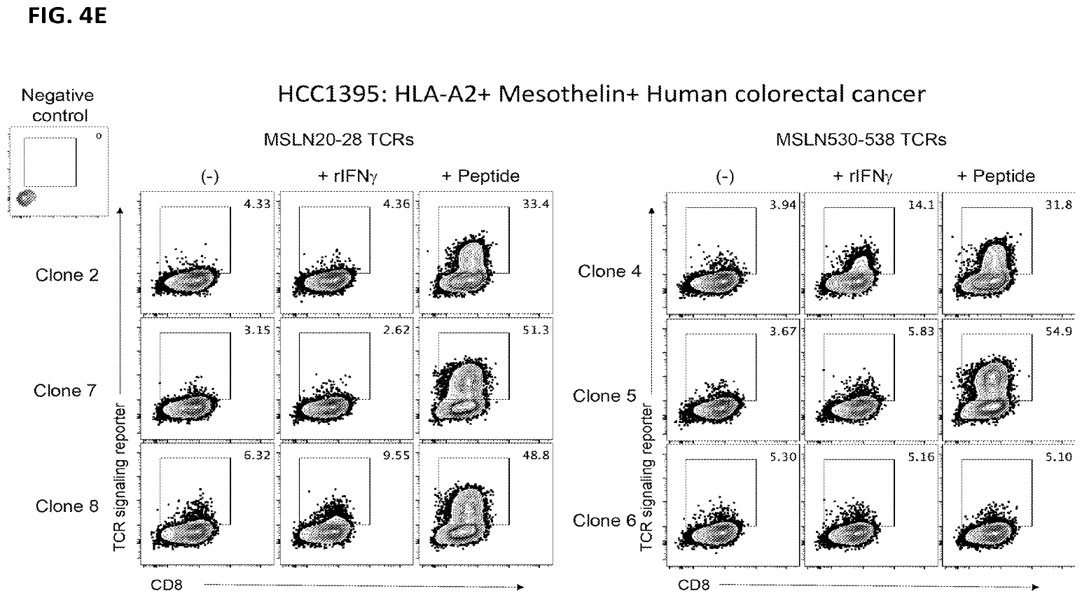

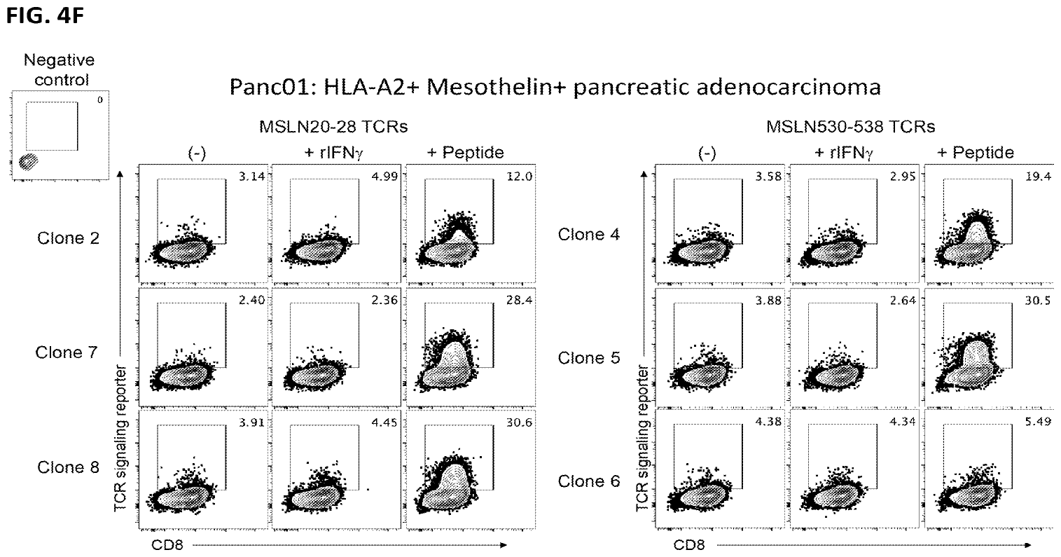

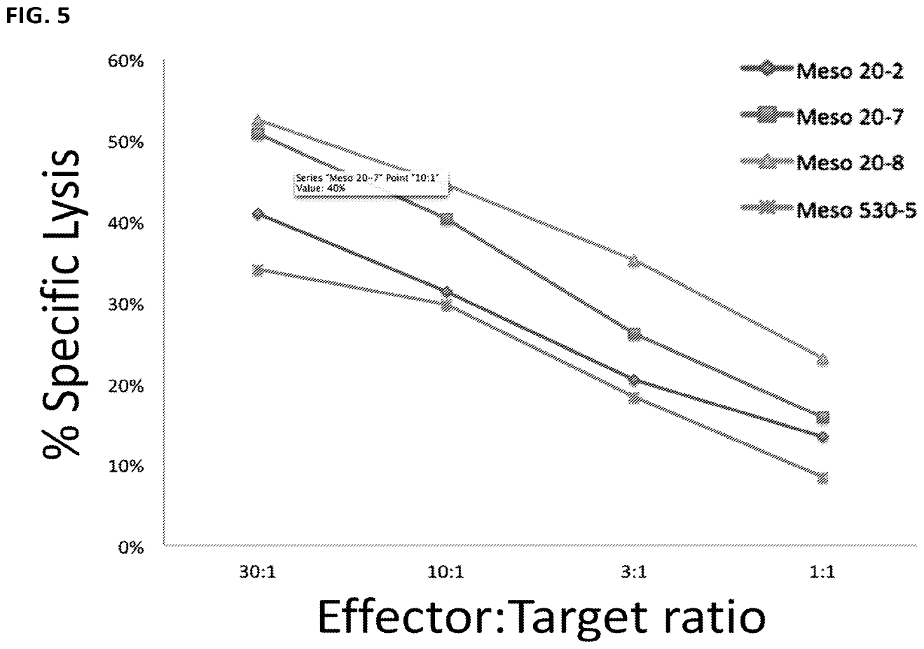

CD8⁺ Jurkat T cells were transduced with the mesothelin-specific TCR clones and analyzed by flow cytometry for tetramer binding. Expression of the codon-optimized mesothelin-specific TCR clones in CD8⁺ Jurkat T cells is shown in FIG. 3 and FIG. 4A-B. Without wishing to be bound by theory, it is believed that differences in tetramer staining intensity may reflect differences in TCR affinity to antigen (mesothelin).

Expression of the codon-optimized mesothelin-specific TCR clones in CD8⁺ or CD8 Jurkat T cells is shown in FIG. 4A. MSLN20 TCRs bound tetramer independent of CD8 coreceptor with consistently higher affinity than MSLN530 TCRs which bind tetramer only in the presence of CD8 coreceptor.

As shown in FIG. 4B, clone 2 and clone 4 stain brightest for tetramer, consistent with higher affinity. As shown in FIG. 4C, three cancer cell lines (HCC1395, OVCAR3 and Panc01) were selected based on a range of expression of HLA-A2 and MSLN for evaluating their ability to induce TCR signaling in MSLN TCR-transduced JURKAT cells. Unexpectedly, clone 8 and clone 5 responded the greatest to all three cancer cell lines tested both in the presence and absence of exogenous MSLN specific peptides (FIG. 4D-F). Panc01 adenocarcinoma cells expressed the lowest levels of HLA and MSLN of the three lines tested (FIG. 4C) and resulted in overall decreased T cell response (FIG. 4F) as compared to HCC1395 (FIG. 4E) and OVCAR3 cancer cells (FIG. 4D). Clone 6 bound tetramer with the weakest affinity (FIG. 4B) and failed to induce robust TCR signaling, even in the presence of exogenous peptide (FIG. 4D-F). Together, these data evidence that tetramer staining, a surrogate for TCR affinity, may not predict TCR cell functionality or sensitivity to tumor antigen. As peptide pulsing tumor cells improves TCR signaling (FIG. 4D-F), antigen is limiting in carcinoma cells; hence, increasing HLA or providing peptide can increase engineered TCR recognition of antigen expressed by tumor cells.

As shown in FIG. 5, MSLN20-28 clone 7 and clone 8 also resulted in increased Panc01 tumor cell lysis compared to the higher affinity TCR MSLN20-28 clone 2. Unexpectedly, MSLN530-538 CLONE 5 exhibited almost similar levels of tumor cell death as compared to the higher affinity TCR MSLN20-28 clone 2 (FIG. 5).

These data suggest that there is an affinity threshold for TCRs that results in more favorable T cell functionality and antitumor activity.

### Example 4—Characterization of In Vivo Antitumor Activity of Human T Cells Expressing Cloned Human TCRs

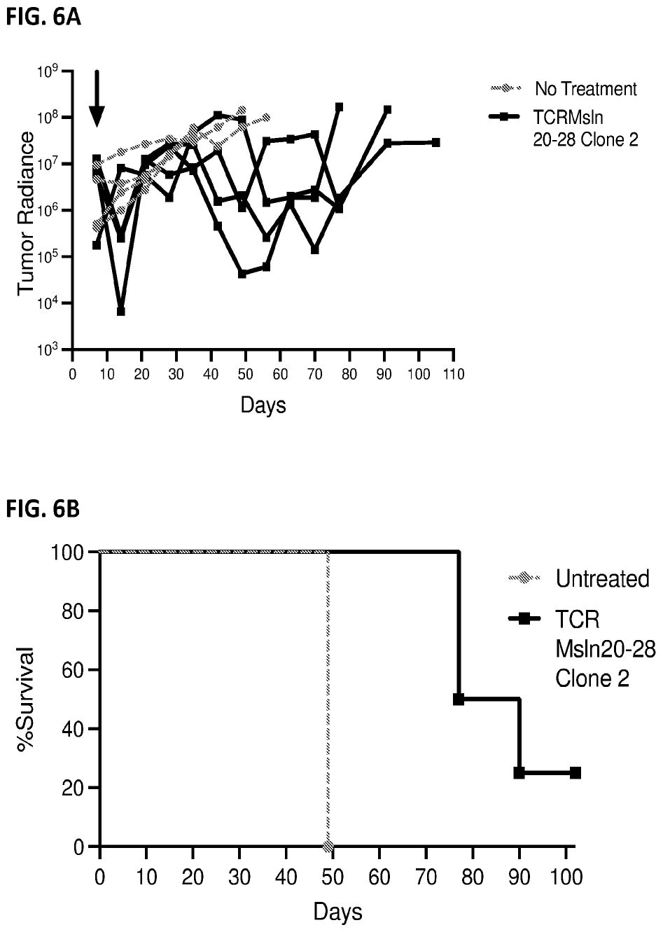

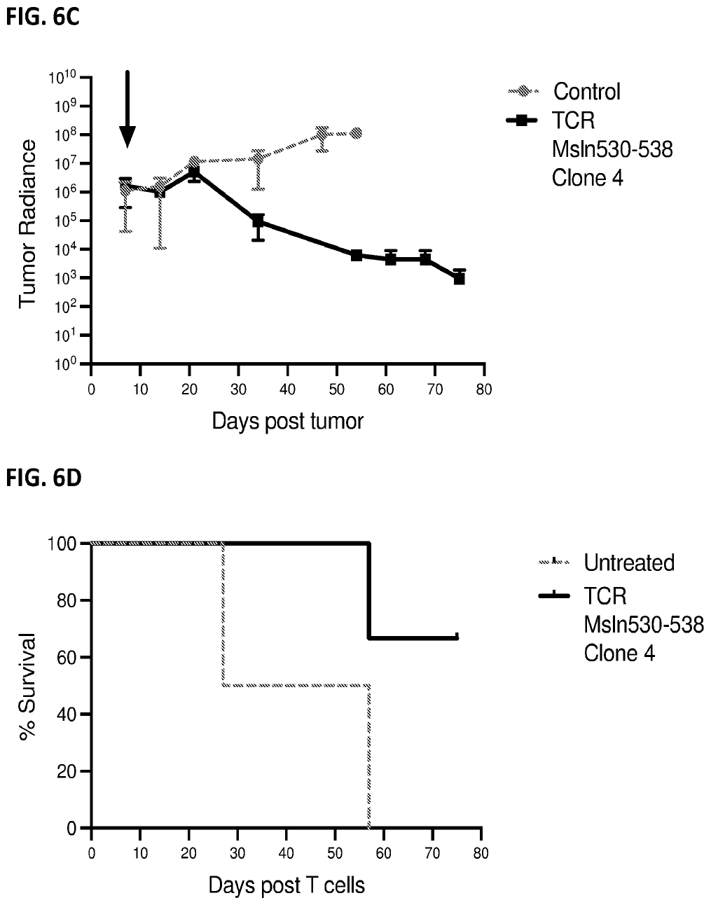

Human primary T cells were genetically modified to express the highest affinity MSLN₂₀₋₂₈ TCR clone 2 in vitro. Immunocompromised NSG mice were orthotopically implanted with Panc01-Luciferase tumor cells (1×10⁶) into the mouse pancreas. On day 7 after tumor implantation, once tumors were established based on IVIS imaging, a total of 5×10⁶ TCR+ T cells were infused i.p. Tumor size was imaged weekly and overall survival was compared to untreated control tumor-bearing mice. FIG. 6A illustrates the antitumor effect of TCR MSLN₂₀₋₂₈ T cells (clone 2) on day 48 post T cell administration; the data are representative of one experiment. FIG. 6B shows a significant prolongation of mouse survival. Experimental endpoint is once tumors have a radiance of >1×10⁸.

A similar experiment was performed as in FIG. 6A-B, but which tested the efficacy of MSLN₅₃₀₋₅₃₈ TCR transduced T cells (clone 4). Less cells were utilized in this study; thus, tumor-bearing mice received only 1×10⁶ T cells. FIG. 6C shows tumor size in the pancreas as determined by bioluminescent imaging. FIG. 6D shows overall mouse survival. Again, experimental endpoint is once tumors have a radiance of >1×10⁸. Experiments reflected in FIG. 6 were performed using the weakly immunogenic Panc01 line (FIG. 4) without a lymphodepletion regimen, without cytokine support, without adjuvant, and without a vaccine, and is a high bar for T cells to produce antitumor activity. Antitumor effects were detected even with the highest affinity T cell clones that were less active in in vitro functionality screens (FIG. 4).

### Example 5—Characterization of In Vivo Antitumor Activity of Mouse T Cells Expressing Cloned Mouse Mesothelin-Specific TCRs

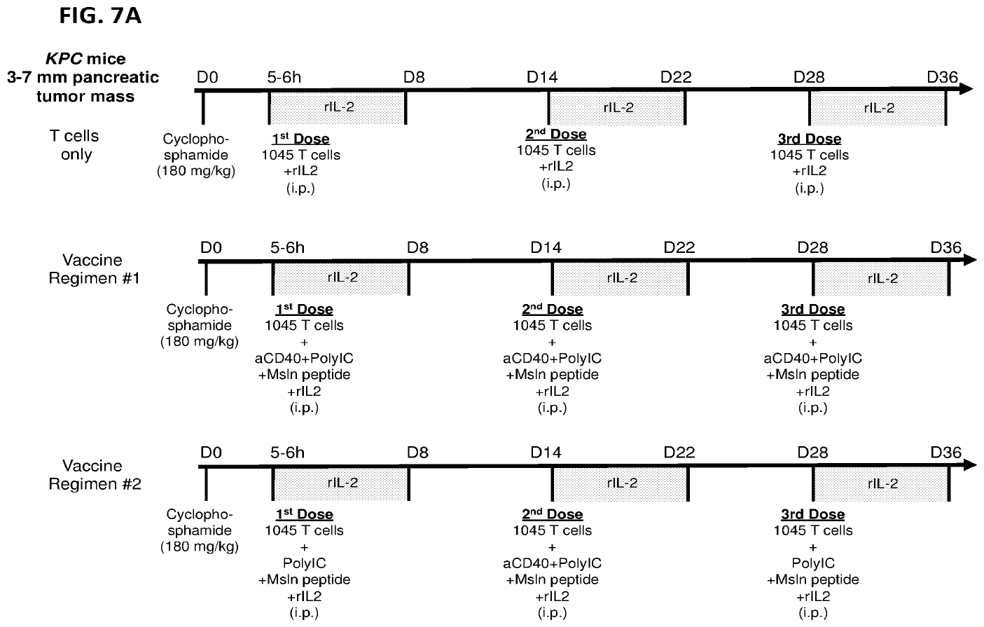

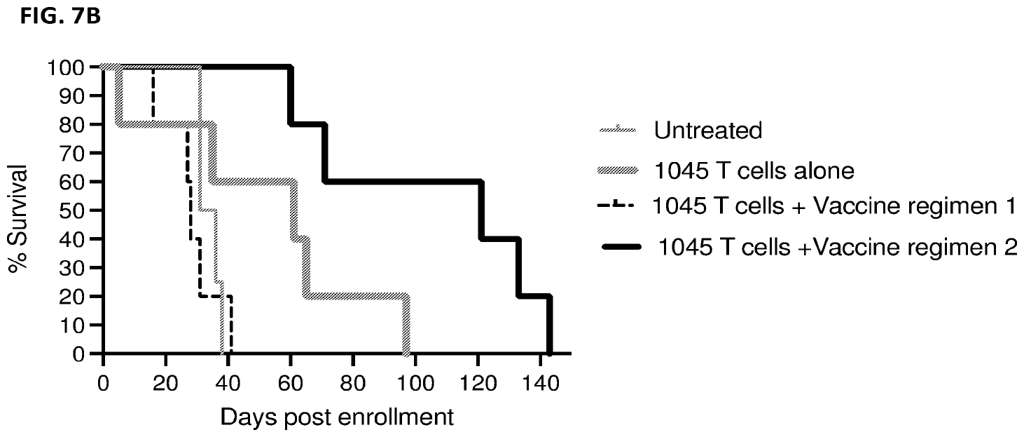

Mouse mesothelin-specific TCRs were prepared that recognize Msln406-414:H-2D^{b}. Evaluation of the clones showed that one TCR (clone 1045) demonstrated significant antitumor activity in highly aggressive syngeneic and immunocompetent mouse models of pancreatic and ovarian cancer. The mouse model utilized was the Kras^{G12D/+}; Trp53^{R172H/+};p48-Cre (KPC) mouse, which is a genetically engineered “spontaneous” model of pancreatic ductal adenocarcinoma (PDA). KPC mice recapitulate the genetics, histological progression, fibroinflammatory tumor microenvironment, metastasis, and therapeutic response as human PDA. The efficacy of 1045 T cells was tested alone and in combination with two different vaccination strategies to enhance antitumor activity (FIG. 7A). KPC mice were enrolled for therapy once they demonstrated invasive PDA (3-7 mm tumor mass as determined by high resolution ultrasound). First, they received cyclophosphamide intraperitoneally (i.p) followed by 5×10⁶ 1045 T cells (i.p). All recipients received recombinant human IL-2 on days 0, 2, 4, 6, and 8 post T cells to promote engineered T cell proliferation. Some recipients received vaccine regimen #1 and another cohort received vaccine regimen #2 (FIG. 7A). This vaccine regimen (CD40 agonist+Poly:IC+peptide) was developed to overcome the obstacle of limiting antigenicity of the tumor cells and the suppressive tumor microenvironment.

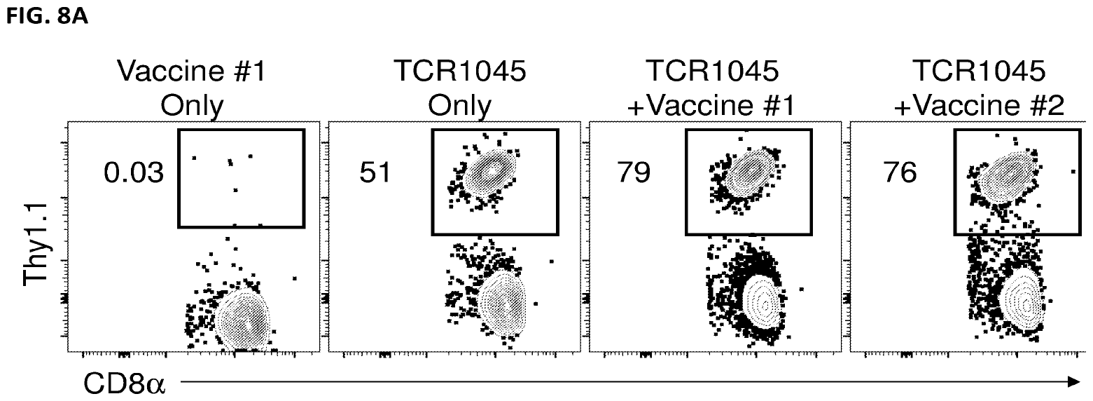

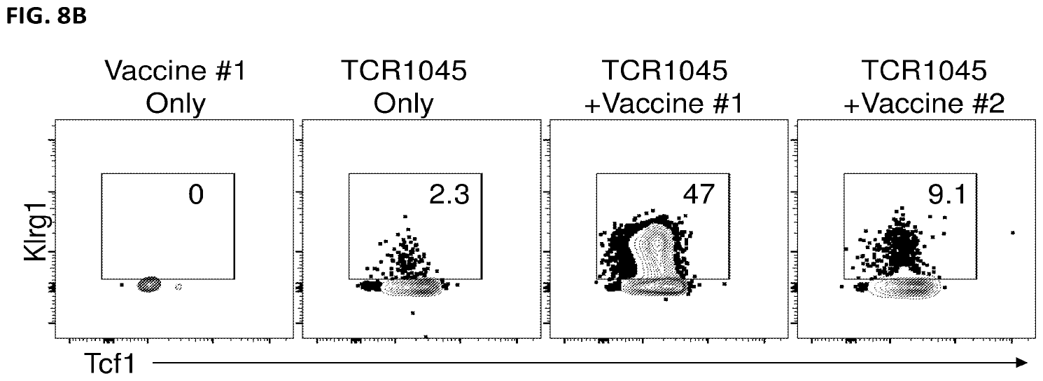

Surprisingly, vaccine regimen #1 in combination with T cell therapy caused toxicity after the 2^{nd} dose (FIG. 7B), thereby abrogating the survival benefit observed with 1045 T cells only (FIG. 7B). Reducing CD40 agonist to be administered only once, as in vaccine regimen #2 with T cell therapy, significantly prolonged KPC survival (p<0.001). Analysis of the transferred engineered T cells in the blood on day 7 post transfer showed that Poly:IC+peptide expanded the transferred T cells to the same extent CD40 agonist+Poly:IC+peptide (FIG. 8A), and both vaccination regimens increased the frequency of donor 1045 T cells (FIG. 8A). Vaccination did not decrease the memory stem cell transcription factor Tef1 by the engineered T cells (FIG. 8B), demonstrating that this approach does not impair long-lived memory formation of the engineered T cells. CD40 agonist+Poly:IC+peptide did increase the frequency of Klrg1+ engineered T cells in circulation (FIG. 8B), which is a surrogate marker for highly potent cytotoxic T cells.

### Example 6—Characterization of In Vivo Antitumor Activity Mouse TCR+T Cells that are Deficient in Tgfbr2

The KPC mouse model is incurable because pancreas progenitor cells continually express mutant oncogenic Kras and mutant Trp53. A syngeneic, immunocompetent orthotopic mouse model was developed in which KPC tumor cells were implanted into the pancreas of syngeneic C57B16/J mice (FIG. 9A). Two scenarios were tested in this model: (i) 1045 T cells and (ii) 1045 T cells in which the suppressive cytokine receptor transforming growth factor beta receptor (Tgfbr2) was edited using a CRISPR/Cas9-based gene editing approach. This KPC orthotopic tumor model is highly aggressive and untreated mice are typically euthanized between 15- and 18-days post tumor implantation due to excessive tumor burden. Therefore, mice were treated with a single dose of vaccine regimen #1+1045 T cells (FIG. 9A). Tumor-bearing mice received 5×10⁶ T cells on day 6 post tumor implantation (˜3-5 mm tumor mass). On day 12, which is 7 days post T cell transfer, tumor weights were recorded. 1045 T cells alone had significant antitumor benefit (FIG. 9B). 1045 T cells defective in Tgfbr2 administered in conjunction with vaccination had the most pronounced antitumor effects (FIG. 9B). Further engineering the T cells described herein to be refractory to suppressive cytokine signaling, when used in combination with vaccine regimen #1, appeared to be safe and the most efficacious regimen tested so far.

The engineered 1045 T cells express a congenic marker Thy1.1 to allow detection following infusion into immunocompetent mice. Removing Tgfβ signaling in 1045 T cells significantly increased the frequency of engineered T cells in the tumor following vaccination (FIG. 9C). Indeed, over 90% of the total CD8+ T cells in the tumor were the 1045 Thy1.1+ T cells in mice that received 1045 Tgfbr2−/−+vaccine regimen #1 (FIG. 9D). Vaccination significantly expanded the frequency (FIG. 9D) and number of genetically engineered T cells systemically and intratumorally (FIG. 9D).

CD69 is a marker that is acutely induced on the surface of T cells following antigen recognition. When Tgfbr2 is deleted, a higher frequency of 1045 T cells express CD69 specifically in the tumor (FIG. 9E-F). These data indicated that interfering with Tgfbr2 on the engineered T cells renders T cells more likely to respond to antigen, which is particularly desirable when targeting carcinomas that have low MHC class I, such as pancreatic cancer.

The complete disclosure of all patents, patent applications, and publications, and electronically available material (including, for instance, nucleotide sequence submissions in, for example, GenBank and RefSeq, and amino acid sequence submissions in, for example, SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

TABLE 1
(MSLN₂₀₋₂₈, clone 2)

| | | |
|---|---|---|
| CDR3 TCRα peptide | CAASGNTDKLIF | SEQ ID NO: 3 |
| CDR3 TCRα DNA | tgt gcc gcc agc ggc aac aac gac aag ctg atc ttt | SEQ ID NO: 15 |
| | (Junction length: 36 nucleotides) | |
| CDR3 TCRβ peptide | CASRPGWSYEQYF | SEQ ID NO: 6 |
| CDR3 TCRβ DNA | tgc gcc agc aga ccc ggc tgg tcc tac gag cag tat ttc | SEQ ID NO: 16 |
| Codon optimized | ATGGATACTTGGCTCGTGTGCTGGGCCATCTTCAGCCTGCTGAAGGCCGGACTGACCGAGCCCGA | SEQ ID NO: 17 |
| (CO) Vbeta | AGTGACCCAGACACCTAGCCACCAAGTGACACAGATGGGCCAGGAAGTGATCCTGCGCTGCGTGC | |
| (DNA) | CCATCAGCAACCACCTGTACTTCTACTGGTACAGACAGATCCTGGGGCAGAAAGTGGAATTCCTG | |
| | GTGTCCTTCTACAACAACGAGATCAGCGAGAAGTCCGAGATCTTCGACGACCAGTTCAGCGTGGA | |
| | ACGGCCCGACGGCAGCAACTTCACCCTGAAGATCAGAAGCACCAAGCTGGAAGATAGCGCCATG | |
| | TACTTTTGCGCCAGCAGACCCGGCTGGTCCTACGAGCAGTATTTCGGCCCTGGCACCCGGCTGACC | |
| | GTGACCGAG | |
| CO VbetaConstant | GATCTGAAGAACGTGTTCCCCCCAGAGGTGGCCGTGTTCGAGCCTTCTGAGGCCGAGATCTCCCA | SEQ ID NO: 18 |
| (DNA) | CACCCAGAAAGCCACCCTCGTGTGTCTGGCCACCGGCTTCTACCCCGACCACGTGGAACTGTCTTG | |
| | GTGGGTCAACGGCAAAGAGGTGCACAGCGGCGTGTCCACCGATCCCCAGCCTCTGAAAGAACAG | |
| | CCCGCCCTGAACGACAGCCGGTACTGCCTGAGCAGCAGACTGAGAGTGTCCGCCACCTTCTGGCA | |
| | GAACCCCCGGAACCACTTCAGATGCCAGGTGCAGTTCTACGGCCTGAGCGAGAACGACGAGTGG | |
| | ACCCAGGATAGAGCCAAGCCCGTGACTCAGATCGTGTCCGCCGAAGCTTGGGGCAGAGCCGATTG | |
| | CGGCTTTACCAGCGAGAGCTACCAGCAGGGCGTGCTGAGCGCCACCATCCTGTACGAGATCCTGC | |
| | TGGGCAAGGCCACCCTGTACGCCGTGCTGGTGTCTGCCCTGGTGCTGATGGCCATGGTCAAGCGG | |
| | AAGGACAGCAGAGGC | |
| P2A | GGTTCCGGAGCCACGAACTTCTCTCTGTTAAAGCAAGCAGGAGACGTGGAAGAAAACCCCGGTCC | SEQ ID NO: 19 |
| (DNA) | C | |
| CO Valpha | ATGGCTATGCTGCTGGGCGCCTCTGTGCTGATCCTGTGGCTGCAGCCCGACTGGGTCAACAGCCA | SEQ ID NO: 20 |
| (DNA) | GCAGAAGAATGACGACCAGCAAGTGAAGCAGAACTCCCCCAGCCTGAGCGTGCAGGAAGGCAGA | |
| | ATCAGCATCCTGAACTGCGACTACACCAACTCTATGTTCGACTACTTCCTGTGGTACAAGAAGTAC | |
| | CCCGCCGAGGGCCCCACCTTCCTGATCAGCATCAGCAGCATCAAGGACAAGAACGAGGACGGCC | |
| | GGTTCACCGTGTTTCTGAACAAGAGCGCCAAGCACCTGAGCCTGCACATCGTGCCTAGCCAGCCT | |
| | GGCGATTCCGCCGTGTATTTCTGTGCCGCCAGCGGCAACACCGACAAGCTGATCTTTGGCACCGG | |
| | CACCAGACTGCAGGTGTTCCCCAAC | |
| CO Valpha Constant | ATCCAGAACCCCGACCCTGCCGTGTACCAGCTGAGAGACAGCAAGAGCAGCGACAAGAGCGTGT | SEQ ID NO: 21 |
| (DNA) | GCCTGTTCACCGACTTCGACAGCCAGACCAACGTGTCCCAGAGCAAGGACAGCGACGTGTACATC | |
| | ACCGATAAGACCGTGCTGGACATGCGGAGCATGGACTTCAAGAGCAACAGCGCCGTGGCCTGGTC | |
| | CAACAAGTCCGATTTCGCCTGCGCCAACGCCTTCAACAACAGCATTATCCCCGAGGACACATTCTT | |
| | CCCAAGCCCCGAGAGCAGCTGCGACGTGAAGCTGGTGGAAAAGAGCTTCGAGACAGACACCAAC | |
| | CTGAACTTCCAGAACCTGTCCGTGATCGGCTTCAGAATCCTGCTGCTGAAAGTGGCCGGCTTCAAT | |
| | CTGCTGATGACCCTGCGGCTGTGGTCCAGCTGA | |
| CO-Vbeta-Vbeta | ATGGATACTTGGCTCGTGTGCTGGGCCATCTTCAGCCTGCTGAAGGCCGGACTGACCGAGCCCGA | SEQ ID NO: 22 |
| constant-P2A- | AGTGACCCAGACACCTAGCCACCAAGTGACACAGATGGGCCAGGAAGTGATCCTGCGCTGCGTGC | |
| Valpha-Valpha | CCATCAGCAACCACCTGTACTTCTACTGGTACAGACAGATCCTGGGGCAGAAAGTGGAATTCCTG | |
| constant | GTGTCCTTCTACAACAACGAGATCAGCGAGAAGTCCGAGATCTTCGACGACCAGTTCAGCGTGGA | |
| (DNA) | ACGGCCCGACGGCAGCAACTTCACCCTGAAGATCAGAAGCACCAAGCTGGAAGATAGCGCCATG | |
| | TACTTTTGCGCCAGCAGACCCGGCTGGTCCTACGAGCAGTATTTCGGCCCTGGCACCCGGCTGACC | |
| | GTGACCGAGGATCTGAAGAACGTGTTCCCCCCAGAGGTGGCCGTGTTCGAGCCTTCTGAGGCCGA | |
| | GATCTCCCACACCCAGAAAGCCACCCTCGTGTGTCTGGCCACCGGCTTCTACCCCGACCACGTGG | |
| | AACTGTCTTGGTGGGTCAACGGCAAAGAGGTGCACAGCGGCGTGTCCACCGATCCCCAGCCTCTG | |
| | AAAGAACAGCCCGCCCTGAACGACAGCCGGTACTGCCTGAGCAGCAGACTGAGAGTGTCCGCCA | |
| | CCTTCTGGCAGAACCCCCGGAACCACTTCAGATGCCAGGTGCAGTTCTACGGCCTGAGCGAGAAC | |
| | GACGAGTGGACCCAGGATAGAGCCAAGCCCGTGACTCAGATCGTGTCCGCCGAAGCTTGGGGCA | |
| | GAGCCGATTGCGGCTTTACCAGCGAGAGCTACCAGCAGGGCGTGCTGAGCGCCACCATCCTGTACGAG | |
| | ATCCTGCTGGGCAAGGCCACCCTGTACGCCGTGCTGGTGTCTGCCCTGGTGCTGATGGCCATGGTCAA | |
| | GCGGAAGGACAGCAGAGGCggttccggagccacgaacttctctctgttaaagcaagcaggagacgtgg | |
| | aagaaaaccccggtcccATGGCTATGCTGCTGGGCGCCTCTGTGCTGATCCTGTGGCTGCAGCCCGAC | |
| | TGGGTCAACAGCCAGCAGAAGAATGACGACCAGCAAGTGAAGCAGAACTCCCCCAGCCTGAGCGTGCA | |
| | GGAAGGCAGAATCAGCATCCTGAACTGCGACTACACCAACTCTATGTTCGACTACTTCCTGTGGTACA | |
| | AGAAGTACCCCGCCGAGGGCCCCACCTTCCTGATCAGCATCAGCAGCATCAAGGACAAGAACGAGGAC | |
| | GGCCGGTTCACCGTGTTTCTGAACAAGAGCGCCAAGCACCTGAGCCTGCACATCGTGCCTAGCCA | |
| | GCCTGGCGATTCCGCCGTGTATTTCTGTGCCGCCAGCGGCAACACCGACAAGCTGATCTTTGGCAC | |
| | CGGCACCAGACTGCAGGTGTTCCCCAACATCCAGAACCCCGACCCTGCCGTGTACCAGCTGAGAG | |
| | ACAGCAAGAGCAGCGACAAGAGCGTGTGCCTGTTCACCGACTTCGACAGCCAGACCAACGTGTCC | |
| | CAGAGCAAGGACAGCGACGTGTACATCACCGATAAGACCGTGCTGGACATGCGGAGCATGGACT | |
| | TCAAGAGCAACAGCGCCGTGGCCTGGTCCAACAAGTCCGATTTCGCCTGCGCCAACGCCTTCAAC | |
| | AACAGCATTATCCCCGAGGACACATTCTTCCCAAGCCCCGAGAGCAGCTGCGACGTGAAGCTGGT | |
| | GGAAAAGAGCTTCGAGACAGACACCAACCTGAACTTCCAGAACCTGTCCGTGATCGGCTTCAGAA | |
| | TCCTGCTGCTGAAAGTGGCCGGCTTCAATCTGCTGATGACCCTGCGGCTGTGGTCCAGCTGA | |
| Valpha | MAMLLGASVLILWLQPDWVNSQQKNDDQQVKQNSPSLSVQEGRISILNCDYTNSMFDYFLWYKKYP | SEQ ID NO: 23 |
| (Peptide) | AEGPTFLISISSIKDKNEDGRFTVFLNKSAKHLSLHIVPSQPGDSAVYFCAASGNTDKLIFGTGTRLQV | |
| | FPN | |
| Valpha constant | IQNPDPAVYQLRDSKSSDKSVCLFTDFDSQTNVSQSKDSDVYITDKTVLDMRSMDFKSNSAVAWSNK | SEQ ID NO: 24 |
| (Peptide) | SDFACANAFNNSIIPEDTFFPSPESSCDVKLVEKSFETDTNLNFQNLSVIGFRILLLKVAGFNLLMTLRL | |
| | WSS | |
| Valpha- | MAMLLGASVLILWLQPDWVNSQQKNDDQQVKQNSPSLSVQEGRISILNCDYTNSMFDYFLWYK | SEQ ID NO: 25 |
| Valpha constant | KYPAEGPTFLISISSIKDKNEDGRFTVFLNKSAKHLSLHIVPSQPGDSAVYFCAASGNTDKLIFGTG | |
| (Peptide) | TRLQVFPNIQNPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILLLKVAGFN | |
| | LLMTLRLWSS | |
| Vbeta | MDTWLVCWAIFSLLKAGLTEPEVTQTPSHQVTQMGQEVILRCVPISNHLYFYWYRQILGQKVEFLVSF | SEQ ID NO: 26 |
| (Peptide) | YNNEISEKSEIFDDQFSVERPDGSNFTLKIRSTKLEDSAMYFCASRPGWSYEQYFGPGTRLTVTE | |
| Vbeta constant | DLKNVFPPEVAVFEPSEAEISHTQKATLVCLATGFYPDHVELSWWVNGKEVHSGVSTDPQPLKEQPAL | SEQ ID NO: 27 |
| (Peptide) | NDSRYCLSSRLRVSATFWQNPRNHFRCQVQFYGLSENDEWTQDRAKPVTQIVSAEAWGRADCGFTSE | |
| | SYQQGVLSATILYEILLGKATLYAVLVSALVLMAMVKRKDSRG | |
| Vbeta- | MDTWLVCWAIFSLLKAGLTEPEVTQTPSHQVTQMGQEVILRCVPISNHLYFYWYRQILGQKVE | SEQ ID NO: 28 |
| Vbeta constant | FLVSFYNNEISEKSEIFDDQFSVERPDGSNFTLKIRSTKLEDSAMYFCASRPGWSYEQYFGPGTRLT | |
| (Peptide) | VTEDLKNVFPPEVAVFEPSEAEISHTQKATLVCLATGFYPDHVELSWWVNGKEVHSGVSTDPQPLKE | |
| | QPALNDSRYCLSSRLRVSATFWQNPRNHFRCQVQFYGLSENDEWTQDRAKPVTQIVSAEAWGRADC | |
| | GFTSESYQQGVLSATILYEILLGKATLYAVLVSALVLMAMVKRKDSRG | |
| Vbeta-Vbeta | MDTWLVCWAIFSLLKAGLTEPEVTQTPSHQVTQMGQEVILRCVPISNHLYFYWYRQILGQKVE | SEQ ID NO: 29 |
| constant-P2A- | FLVSFYNNEISEKSEIFDDQFSVERPDGSNFTLKIRSTKLEDSAMYFCASRPGWSYEQYFGPGTRLT | |
| Valpha-Valpha | VTEDLKNVFPPEVAVFEPSEAEISHTQKATLVCLATGFYPDHVELSWWVNGKEVHSGVSTDPQPLKE | |
| constant | QPALNDSRYCLSSRLRVSATFWQNPRNHFRCQVQFYGLSENDEWTQDRAKPVTQIVSAEAWGRADC | |
| (Peptide) | GFTSESYQQGVLSATILYEILLGKATLYAVLVSALVLMAMVKRKDSRGGSGATNFSLLKQAGDVEENP | |
| <Full Length TCR | GPMAMLLGASVLILWLQPDWVNSQQKNDDQQVKQNSPSLSVQEGRISILNCDYTNSMFDYFLW | |
| sequence> | YKKYPAEGPTFLISISSIKDKNEDGRFTVFLNKSAKHLSLHIVPSQPGDSAVYFCAASGNTDKLIFG | |
| | TGTRLQVFPNIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGFRILLLKVA | |
| | GFNLLMTLRLWSS | |

TABLE 2
(MSLN₂₀₋₂₈, clone 7)

| | | |
|---|---|---|
| CDR3 TCRα peptide | CAFYMDSNYQLIW | SEQ ID NO: 4 |
| CDR3 TCRα DNA | tgc gcc ttc tac atg gac agc aac tac cag ctg atc tgg | SEQ ID NO: 30 |
| CDR3 TCRβ peptide | CASSEWTAEQYF | SEQ ID NO: 7 |
| CDR3 TCRβ DNA | tgt gcc agc agc gag tgg acc gcc gag cag tat ttt | SEQ ID NO: 31 |
| CO-Vbeta-Vbeta | TGGCGCGCCACCATGTCTATCGGCCTGCTGTGCTGCGTGGCCTTCAGTCTGCTGTGGGCCAGCCCTG | SEQ ID NO: 32 |
| constant-P2A- | TGAATGCCGGCGTGACCCAGACCCCCAAGTTCCAGGTGCTGAAAACCGGCCAGAGCATGACCCTGC | |
| Valpha-Valpha | AGTGCGCCCAGGACATGAACCACAACAGCATGTACTGGTACAGACAGGACCCCGGCATGGGCCTGC | |
| constant | GGCTGATCTACTACTCTGCCAGCGAGGGCACCACCGACAAGGGCGAAGTGCCCAACGGCTACAACG | |
| (DNA) | TGTCCCGGCTGAACAAGAGAGAGTTCAGCCTGAGACTGGAAAGCGCCGCTCCCAGCCAGACCAGCG | |
| | TGTACTTTTGTGCCAGCAGCGAGTGGACCGCCGAGCAGTATTTTGGCCCTGGCACCAGACTGACCGT | |
| | GACCGAGGACCTGAAGAACGTGTTCCCCCCAGAGGTGGCCGTGTTCGAGCCTTCTGAGGCCGAGAT | |
| | CAGCCACACCCAGAAAGCCACCCTCGTGTGTCTGGCCACCGGCTTCTACCCCGACCACGTGGAACT | |
| | GTCTTGGTGGGTCAACGGCAAAGAGGTGCACAGCGGCGTGTCCACCGATCCCCAGCCTCTGAAAGA | |
| | ACAGCCCGCCCTGAACGACAGCCGGTACTGCCTGTCCAGCAGGCTGAGAGTGTCCGCCACCTTCTG | |
| | GCAGAACCCCCGGAACCACTTCAGATGCCAGGTGCAGTTCTACGGCCTGAGCGAGAACGATGAGTG | |
| | GACCCAGGACAGAGCCAAGCCCGTGACACAGATCGTGTCTGCCGAAGCCTGGGGCAGAGCCGATT | |
| | GCGGCTTTACCAGCGAGAGCTACCAGCAGGGCGTGCTGAGCGCCACAATCCTGTACGAGATCCTGC | |
| | TGGGCAAGGCCACCCTGTACGCCGTGCTGGTGTCTGCCCTGGTGCTGATGGCCATGGTCAAGCGGA | |
| | AGGACAGCAGAGGCGGTTCCGGAGCCACGAACTTCTCTCTGTTAAAGCAAGCAGGAGACGTGGAAGAAA | |
| | ACCCCGGTCCCATGGAAAAGAACCCCCTGGCCGCTCCCCTGCTGATCCTGTGGTTTCACCTGGACTG | |
| | CGTGTCCTCCATCCTGAACGTGGAACAGAGCCCCCAGAGCCTGCATGTGCAGGAAGGCGACAGCAC | |
| | CAACTTCACCTGTAGCTTCCCCAGCAGCAACTTCTACGCCCTGCACTGGTATAGATGGGAGACAGCC | |
| | AAGAGCCCCGAGGCCCTGTTCGTGATGACCCTGAACGGCGACGAGAAGAAGAAGGGCCGGATCAG | |
| | CGCCACCCTGAACACCAAAGAGGGCTACAGCTACCTGTACATCAAGGGCAGCCAGCCCGAGGACA | |
| | GCGCCACATACCTGTGCGCCTTCTACATGGACAGCAACTACCAGCTGATCTGGGGAGCCGGCACCA | |
| | AGCTGATCATCAAGCCCGACATCCAGAACCCCGACCCCGCCGTGTATCAGCTGAGGGACAGCAAGA | |
| | GCAGCGACAAGAGCGTGTGCCTGTTCACCGACTTCGACTCCCAGACCAATGTGTCCCAGAGCAAGG | |
| | ACAGCGACGTGTACATTACCGACAAGACCGTGCTGGACATGCGGAGCATGGACTTCAAGAGCAACA | |
| | GCGCCGTGGCCTGGTCCAACAAGAGCGATTTCGCCTGCGCCAACGCCTTCAACAACTCCATTATCCC | |
| | TGAGGACACATTCTTCCCAAGCCCCGAGAGCAGCTGCGACGTGAAGCTGGTGGAAAAGAGCTTCGA | |
| | GACAGACACCAACCTGAACTTCCAGAACCTGAGCGTGATCGGCTTCAGAATCCTGCTGCTGAAGGT | |
| | GGCCGGCTTCAACCTGCTGATGACACTGCGGCTGTGGTCCAGCTGAGTCGAC | |
| Valpha | MEKNPLAAPLLILWFHLDCVSSILNVEQSPQSLHVQEGDSTNFTCSFPSSNFYALHWYRWETAKSPEALF | SEQ ID NO: 33 |
| (Peptide) | VMTLNGDEKKKGRISATLNTKEGYSYLYIKGSQPEDSATYLCAFYMDSNYQLIWGAGTKLIIKPD | |
| Valpha constant | IQNPDPAVYQLRDSKSSDKSVCLFTDFDSQTNVSQSKDSDVYITDKTVLDMRSMDFKSNSAVAWSNKSDF | SEQ ID NO: 34 |
| (Peptide) | ACANAFNNSIIPEDTFFPSPESSCDVKLVEKSFETDTNLNFQNLSVIGFRILLLKVAGFNLLMTLRLWSS | |
| Valpha- | MEKNPLAAPLLILWFHLDCVSSILNVEQSPQSLHVQEGDSTNFTCSFPSSNFYALHWYRWETAKSP | SEQ ID NO: 35 |
| Valpha constant | EALFVMTLNGDEKKKGRISATLNTKEGYSYLYIKGSQPEDSATYLCAFYMDSNYQLIWGAGTKLII | |
| (Peptide) | KPDIQNPDPAVYQLRDSKSSDKSVCLFTDFDSQTNVSQSKDSDVYITDKTVLDMRSMDFKSNSAVAWS | |
| | NKSDFACANAFNNSIIPEDTFFPSPESSCDVKLVEKSFETDTNLNFQNLSVIGFRILLLKVAGFNLLMTL | |
| | RLWSS | |
| Vbeta | MSIGLLCCVAFSLLWASPVNAGVTQTPKFQVLKTGQSMTLQCAQDMNHNSMYWYRQDPGMGLRLIYY | SEQ ID NO: 36 |
| (Peptide) | SASEGTTDKGEVPNGYNVSRLNKREFSLRLESAAPSQTSVYFCASSEWTAEQYFGPGTRLTVTE | |
| Vbeta constant | DLKNVFPPEVAVFEPSEAEISHTQKATLVCLATGFYPDHVELSWWVNGKEVHSGVSTDPQPLKEQPALN | SEQ ID NO: 37 |
| (Peptide) | DSRYCLSSRLRVSATFWQNPRNHFRCQVQFYGLSENDEWTQDRAKPVTQIVSAEAWGRADCGFTSESY | |
| | QQGVLSATILYEILLGKATLYAVLVSALVLMAMVKRKDSRG | |
| Vbeta- | MSIGLLCCVAFSLLWASPVNAGVTQTPKFQVLKTGQSMTLQCAQDMNHNSMYWYRQDPGMGLR | SEQ ID NO: 38 |
| Vbeta constant | LIYYSASEGTTDKGEVPNGYNVSRLNKREFSLRLESAAPSQTSVYFCASSEWTAEQYFGPGTRLTVT | |
| (Peptide) | EDLKNVFPPEVAVFEPSEAEISHTQKATLVCLATGFYPDHVELSWWVNGKEVHSGVSTDPQPLKEQPAL | |
| | NDSRYCLSSRLRVSATFWQNPRNHFRCQVQFYGLSENDEWTQDRAKPVTQIVSAEAWGRADCGFTSES | |
| | YQQGVLSATILYEILLGKATLYAVLVSALVLMAMVKRKDSRG | |
| Vbeta-Vbeta | MSIGLLCCVAFSLLWASPVNAGVTQTPKFQVLKTGQSMTLQCAQDMNHNSMYWYRQDPGMGLR | SEQ ID NO: 39 |
| constant-P2A- | LIYYSASEGTTDKGEVPNGYNVSRLNKREFSLRLESAAPSQTSVYFCASSEWTAEQYFGPGTRLTVT | |
| Valpha-Valpha | EDLKNVFPPEVAVFEPSEAEISHTQKATLVCLATGFYPDHVELSWWVNGKEVHSGVSTDPQPLKEQPAL | |
| constant | NDSRYCLSSRLRVSATFWQNPRNHFRCQVQFYGLSENDEWTQDRAKPVTQIVSAEAWGRADCGFTSES | |
| (Peptide) | YQQGVLSATILYEILLGKATLYAVLVSALVLMAMVKRKDSRGGSGATNFSLLKQAGDVEENPGPMEKN | |
| <Full Length TCR | PLAAPLLILWFHLDCVSSILNVEQSPQSLHVQEGDSTNFTCSFPSSNFYALHWYRWETAKSPEALFV | |
| sequence> | MTLNGDEKKKGRISATLNTKEGYSYLYIKGSQPEDSATYLCAFYMDSNYQLIWGAGTKLIIKPDIQN | |
| | PDPAVYQLRDSKSSDKSVCLFTDFDSQTNVSQSKDSDVYITDKTVLDMRSMDFKSNSAVAWSNKSDFA | |
| | CANAFNNSIIPEDTFFPSPESSCDVKLVEKSFETDTNLNFQNLSVIGFRILLLKVAGFNLLMTLRLWSS | |

TABLE 3
(MSLN₂₀₋₂₈, clone 8)

| | | |
|---|---|---|
| CDR3 TCRα | CAVIPNNNARLMF | SEQ ID NO: 5 |
| peptide | | |
| CDR3 TCRα DNA | tgc gcc gtg atc ccc aac aac aac gcc cgg ctg atg ttt | SEQ ID NO: 40 |
| CDR3 TCRβ | CASGQGTEAFF | SEQ ID NO: 8 |
| peptide | | |
| CDR3 TCRβ DNA | tgt gcc tct ggc cag gga acc gag gca ttc ttt | SEQ ID NO: 41 |
| CO-Vbeta-Vbeta | TGGCGCGCCACCATGGGATGCAGACTGCTGTGTTGCGCCGTGCTGTGTCTGCTGGGAGCCGTGCCTA | SEQ ID NO: 42 |
| constant-P2A- | TGGAAACCGGCGTGACCCAGACCCCCAGACACCTCGTGATGGGCATGACCAACAAGAAAAGCCTG | |
| Valpha-Valpha | AAGTGCGAGCAGCACCTGGGCCACAACGCCATGTACTGGTACAAGCAGAGCGCCAAGAAACCCCT | |
| constant | GGAACTGATGTTCGTGTACAACTTCAAAGAGCAGACCGAGAACAACAGCGTGCCCAGCAGATTCAG | |
| (DNA) | CCCCGAGTGCCCCAATAGCAGCCACCTGTTTCTGCATCTGCACACCCTGCAGCCCGAGGACAGCGC | |
| | CCTGTATCTGTGTGCCTCTGGCCAGGGAACCGAGGCATTCTTTGGGCAGGGCACCAGACTGACCGT | |
| | GGTGGAAGATCTGAACAAGGTGTTCCCCCCAGAGGTGGCCGTGTTCGAGCCTTCTGAGGCCGAGAT | |
| | CAGCCACACCCAGAAAGCCACCCTCGTGTGCCTGGCCACCGGCTTTTTCCCCGACCACGTGGAACTG | |
| | TCTTGGTGGGTCAACGGCAAAGAGGTGCACAGCGGCGTGTCCACCGATCCCCAGCCTCTGAAAGAA | |
| | CAGCCCGCCCTGAACGACAGCCGGTACTGCCTGAGCAGCAGACTGAGAGTGTCCGCCACCTTCTGG | |
| | CAGAACCCCCGGAACCACTTCAGATGCCAGGTGCAGTTCTACGGCCTGAGCGAGAACGACGAGTGG | |
| | ACCCAGGACAGAGCCAAGCCCGTGACACAGATCGTGTCTGCCGAAGCCTGGGGCAGAGCCGATTGC | |
| | GGCTTTACCAGCGTGTCCTATCAGCAGGGCGTGCTGAGCGCCACAATCCTGTACGAGATCCTGCTGG | |
| | GAAAGGCCACCCTGTATGCAGTGCTGGTGTCCGCCCTGGTGCTGATGGCCATGGTCAAGCGGAAGG | |
| | ACTTCGGTTCCGGAGCCACGAACTTCTCTCTGTTAAAGCAAGCAGGAGACGTGGAAGAAAACCCCGGTCC | |
| | CGTCCCATGCTCCTGCTGCTGGTGCCTGCCTTCCAAGTGATCTTCACCCTGGGCGGCACCAGAGCCC | |
| | AGTCTGTGACCCAGCTGGATAGCCAGGTGCCCGTGTTTGAAGAGGCCCCTGTGGAACTGCGGTGCA | |
| | ACTACAGCAGCTCCGTGTCCGTGTACCTGTTTTGGTACGTGCAGTACCCCAACCAGGGCCTGCAGCT | |
| | GCTGCTGAAGTACCTGAGCGGCAGCACCCTCGTGAAGGGAATCAACGGCTTCGAGGCCGAATTCAA | |
| | CAAGAGCCAGACCAGCTTCCACCTGAGAAAGCCCAGCGTGCACATCAGCGATACCGCCGAGTACTT | |
| | CTGCGCCGTGATCCCCAACAACAACGCCCGGCTGATGTTTGGCGACGGCACACAGCTGGTCGTGAA | |
| | GCCCAACATCCAGAACCCCGACCCCGCCGTGTACCAGCTGAGAGACAGCAAGAGCAGCGACAAGA | |
| | GCGTGTGTCTGTTCACCGACTTCGACTCCCAGACCAACGTGTCCCAGAGCAAGGACAGCGACGTGT | |
| | ACATCACCGACAAGACCGTGCTGGACATGCGGAGCATGGACTTCAAGAGCAACAGCGCCGTGGCCT | |
| | GGTCCAACAAGTCCGATTTCGCCTGCGCCAACGCCTTCAACAACAGCATTATCCCTGAGGACACATT | |
| | CTTCCCAAGCCCCGAGAGCAGCTGCGACGTGAAGCTGGTGGAAAAGAGCTTCGAGACAGACACCA | |
| | ACCTGAACTTCCAGAACCTGAGCGTGATCGGCTTCCGGATCCTGCTGCTGAAAGTGGCCGGCTTCAA | |
| | CCTGCTGATGACCCTGAGACTGTGGTCCAGCTGAGTCGAC | |
| Valpha | MLLLLVPAFQVIFTLGGTRAQSVTQLDSQVPVFEEAPVELRCNYSSSVSVYLFWYVQYPNQGLQLLLKY | SEQ ID NO: 43 |
| (Peptide) | LSGSTLVKGINGFEAEFNKSQTSFHLRKPSVHISDTAEYFCAVIPNNNARLMFGDGTQLVVKPN | |
| Valpha constant | IQNPDPAVYQLRDSKSSDKSVCLFTDFDSQTNVSQSKDSDVYITDKTVLDMRSMDFKSNSAVAWSNKSDF | SEQ ID NO: 44 |
| (Peptide) | ACANAFNNSIIPEDTFFPSPESSCDVKLVEKSFETDTNLNFQNLSVIGFRILLLKVAGFNLLMTLRLWSS | |
| Valpha- | MLLLLVPAFQVIFTLGGTRAQSVTQLDSQVPVFEEAPVELRCNYSSSVSVYLFWYVQYPNQGLQLL | SEQ ID NO: 45 |
| Valpha constant | LKYLSGSTLVKGINGFEAEFNKSQTSFHLRKPSVHISDTAEYFCAVIPNNNARLMFGDGTQLVVKPN | |
| (Peptide) | IQNPDPAVYQLRDSKSSDKSVCLFTDFDSQTNVSQSKDSDVYITDKTVLDMRSMDFKSNSAVAWSNKSDF | |
| | ACANAFNNSIIPEDTFFPSPESSCDVKLVEKSFETDTNLNFQNLSVIGFRILLLKVAGFNLLMTLRLWSS | |
| Vbeta | MGCRLLCCAVLCLLGAVPMETGVTQTPRHLVMGMTNKKSLKCEQHLGHNAMYWYKQSAKKPLELMF | SEQ ID NO: 46 |
| (Peptide) | VYNFKEQTENNSVPSRFSPECPNSSHLFLHLHTLQPEDSALYLCASGQGTEAFFGQGTRLTVVE | |
| Vbeta constant | DLNKVFPPEVAVFEPSEAEISHTQKATLVCLATGFFPDHVELSWWVNGKEVHSGVSTDPQPLKEQPALN | SEQ ID NO: 47 |
| (Peptide) | DSRYCLSSRLRVSATFWQNPRNHFRCQVQFYGLSENDEWTQDRAKPVTQIVSAEAWGRADCGFTSVSY | |
| | QQGVLSATILYEILLGKATLYAVLVSALVLMAMVKRKDF | |
| Vbeta- | MGCRLLCCAVLCLLGAVPMETGVTQTPRHLVMGMTNKKSLKCEQHLGHNAMYWYKQSAKKPL | SEQ ID NO: 48 |
| Vbeta constant | ELMFVYNFKEQTENNSVPSRFSPECPNSSHLFLHLHTLQPEDSALYLCASGQGTEAFFGQGTRLTVV | |
| (Peptide) | EDLNKVFPPEVAVFEPSEAEISHTQKATLVCLATGFFPDHVELSWWVNGKEVHSGVSTDPQPLKEQPAL | |
| | NDSRYCLSSRLRVSATFWQNPRNHFRCQVQFYGLSENDEWTQDRAKPVTQIVSAEAWGRADCGFTSVS | |
| | YQQGVLSATILYEILLGKATLYAVLVSALVLMAMVKRKDF | |
| Vbeta-Vbeta | MGCRLLCCAVLCLLGAVPMETGVTQTPRHLVMGMTNKKSLKCEQHLGHNAMYWYKQSAKKPL | SEQ ID NO: 49 |
| constant-P2A- | ELMFVYNFKEQTENNSVPSRFSPECPNSSHLFLHLHTLQPEDSALYLCASGQGTEAFFGQGTRLTVV | |
| Valpha-Valpha | EDLNKVFPPEVAVFEPSEAEISHTQKATLVCLATGFFPDHVELSWWVNGKEVHSGVSTDPQPLKEQPAL | |
| constant | NDSRYCLSSRLRVSATFWQNPRNHFRCQVQFYGLSENDEWTQDRAKPVTQIVSAEAWGRADCGFTSVS | |
| (Peptide) | YQQGVLSATILYEILLGKATLYAVLVSALVLMAMVKRKDFGSGATNFSLLKQAGDVEENPGPMLLLLVP | |
| <Full Length TCR | AFQVIFTLGGTRAQSVTQLDSQVPVFEEAPVELRCNYSSSVSVYLFWYVQYPNQGLQLLLKYLSGS | |
| sequence> | TLVKGINGFEAEFNKSQTSFHLRKPSVHISDTAEYFCAVIPNNNARLMFGDGTQLVVKPNIQNPDPA | |
| | VYQLRDSKSSDKSVCLFTDFDSQTNVSQSKDSDVYITDKTVLDMRSMDFKSNSAVAWSNKSDFACANA | |
| | FNNSIIPEDTFFPSPESSCDVKLVEKSFETDTNLNFQNLSVIGFRILLLKVAGFNLLMTLRLWSS | |

TABLE 4
(MSLN₅₃₀₋₅₃₈ clone 4)

| | | |
|---|---|---|
| CDR3 TCRα | CAYLGTGTYKYIF | SEQ ID NO: 9 |
| peptide | | |
| CDR3 TCRα | tgt gcc tac ctg ggc acc ggc acc tac aag tac atc ttc | SEQ ID NO: 50 |
| DNA | | |
| CDR3 TCRβ | CASSSGGLGYTF | SEQ ID NO: 12 |
| peptide | | |
| CDR3 TCRβ | tgc gcc tct agc tct ggc ggc ctg gga tac aca ttt | SEQ ID NO: 51 |
| DNA | | |
| Codon optimized | ATGGGACCTCAGCTGCTGGGATACGTGGTGCTGTGTCTGCTGGGAGCCGGACCTCTGGAAGCCCAAG | SEQ ID NO: 52 |
| (CO) Vbeta | TGACCCAGAACCCCAGATACCTGATCACCGTGACCGGCAAGAAACTGACCGTGACCTGCAGCCAGA | |
| (DNA) | ACATGAACCACGAGTACATGAGCTGGTACAGACAGGACCCCGGCCTGGGCCTGCGGCAGATCTACT | |
| | ACAGCATGAACGTGGAAGTGACCGACAAGGGCGACGTGCCCGAGGGCTACAAGGTGTCCCGGAAAG | |
| | AGAAGCGGAACTTCCCACTGATCCTGGAAAGCCCCAGCCCCAACCAGACCAGCCTGTACTTCTGCGC | |
| | CTCTAGCTCTGGCGGCCTGGGATACACATTTGGCAGCGGCACCAGGCTGACCGTGGTGGAA | |
| CO Vbeta | GATCTGAACAAGGTGTTCCCCCCAGAGGTGGCCGTGTTCGAGCCTTCTGAGGCCGAGATCAGCCACA | SEQ ID NO: 53 |
| constant | CCCAGAAAGCCACCCTCGTGTGCCTGGCCACCGGCTTTTTCCCCGACCACGTGGAACTGTCTTGGTGG | |
| (DNA) | GTCAACGGCAAAGAGGTGCACAGCGGCGTGTCCACCGATCCCCAGCCTCTGAAAGAACAGCCCGCC | |
| | CTGAACGACAGCCGGTACTGCCTGAGCAGCAGACTGAGAGTGTCCGCCACCTTCTGGCAGAACCCCC | |
| | GGAACCACTTCAGATGCCAGGTGCAGTTCTACGGCCTGAGCGAGAACGACGAGTGGACCCAGGACA | |
| | GAGCCAAGCCCGTGACACAGATCGTGTCTGCCGAAGCCTGGGGCAGAGCCGATTGCGGCTTTACCAG | |
| | CGTGTCCTATCAGCAGGGCGTGCTGAGCGCCACAATCCTGTACGAGATCCTGCTGGGCAAGGCCACC | |
| | CTGTACGCCGTGCTGGTGTCCGCTCTGGTGCTGATGGCCATGGTCAAGCGGAAGGACTTC | |
| P2A | GGTTCCGGAGCCACGAACTTCTCTCTGTTAAAGCAAGCAGGAGACGTGGAAGAAAACCCCGGTCCC | SEQ ID NO: 54 |
| (DNA) | | |
| CO Valpha | ATGGCCTGCCCCGGATTTCTGTGGGCCCTCGTGATCAGCACCTGTCTGGAATTCAGCATGGCCCAGA | SEQ ID NO: 55 |
| (DNA) | CCGTGACTCAGTCCCAGCCCGAGATGAGCGTGCAGGAAGCCGAGACAGTGACCCTGAGCTGCACCT | |
| | ACGACACCAGCGAGAGCGACTACTACCTGTTCTGGTACAAGCAGCCCCCCAGCCGGCAGATGATCCT | |
| | CGTGATTAGACAGGAAGCCTATAAGCAGCAGAACGCCACCGAGAACAGATTCAGCGTGAACTTCCA | |
| | GAAGGCCGCCAAGTCCTTCAGCCTGAAGATCAGCGACAGCCAGCTGGGCGACGCCGCCATGTACTTT | |
| | TGTGCCTACCTGGGCACCGGCACCTACAAGTACATCTTCGGCACAGGCACCCGGCTGAAGGTGCTGG | |
| | CCAAC | |
| CO Valpha | ATCCAGAACCCTGACCCCGCCGTGTATCAGCTGCGGGACAGCAAGAGCAGCGACAAGAGCGTGTGT | SEQ ID NO: 56 |
| constant | CTGTTCACCGACTTCGACTCCCAGACCAACGTGTCCCAGAGCAAGGACAGCGACGTGTACATCACCG | |
| (DNA) | ACAAGACCGTGCTGGACATGCGGAGCATGGACTTCAAGAGCAACAGCGCCGTGGCCTGGTCCAACA | |
| | AGAGCGATTTCGCCTGCGCCAACGCCTTCAACAACAGCATTATCCCCGAGGACACATTCTTCCCAAG | |
| | CCCCGAGAGCAGCTGCGACGTGAAGCTGGTGGAAAAGAGCTTCGAGACAGACACCAACCTGAATTT | |
| | CCAGAACCTGAGCGTGATCGGCTTCAGAATCCTGCTGCTGAAAGTGGCCGGCTTCAACCTGCTGATG | |
| | ACCCTGCGGCTGTGGTCCAGCTG | |
| CO-Vbeta-Vbeta | ATGGGACCTCAGCTGCTGGGATACGTGGTGCTGTGTCTGCTGGGAGCCGGACCTCTGGAAGCCCAAG | SEQ ID NO: 57 |
| constant-P2A- | TGACCCAGAACCCCAGATACCTGATCACCGTGACCGGCAAGAAACTGACCGTGACCTGCAGCCAGA | |
| Valpha-Valpha | ACATGAACCACGAGTACATGAGCTGGTACAGACAGGACCCCGGCCTGGGCCTGCGGCAGATCTACT | |
| constant | ACAGCATGAACGTGGAAGTGACCGACAAGGGCGACGTGCCCGAGGGCTACAAGGTGTCCCGGAAAG | |
| (DNA) | AGAAGCGGAACTTCCCACTGATCCTGGAAAGCCCCAGCCCCAACCAGACCAGCCTGTACTTCTGCGC | |
| | CTCTAGCTCTGGCGGCCTGGGATACACATTTGGCAGCGGCACCAGGCTGACCGTGGTGGAAGATCTG | |
| | AACAAGGTGTTCCCCCCAGAGGTGGCCGTGTTCGAGCCTTCTGAGGCCGAGATCAGCCACACCCAGA | |
| | AAGCCACCCTCGTGTGCCTGGCCACCGGCTTTTTCCCCGACCACGTGGAACTGTCTTGGTGGGTCAAC | |
| | GGCAAAGAGGTGCACAGCGGCGTGTCCACCGATCCCCAGCCTCTGAAAGAACAGCCCGCCCTGAAC | |
| | GACAGCCGGTACTGCCTGAGCAGCAGACTGAGAGTGTCCGCCACCTTCTGGCAGAACCCCCGGAACC | |
| | ACTTCAGATGCCAGGTGCAGTTCTACGGCCTGAGCGAGAACGACGAGTGGACCCAGGACAGAGCCA | |
| | AGCCCGTGACACAGATCGTGTCTGCCGAAGCCTGGGGCAGAGCCGATTGCGGCTTTACCAGCGTGTC | |
| | CTATCAGCAGGGCGTGCTGAGCGCCACAATCCTGTACGAGATCCTGCTGGGCAAGGCCACCCTGTAC | |
| | GCCGTGCTGGTGTCCGCTCTGGTGCTGATGGCCATGGTCAAGCGGAAGGACTTCGGTTCCGGAGCCAC | |
| | GAACTTCTCTCTGTTAAAGCAAGCAGGAGACGTGGAAGAAAACCCCGGTCCCATGGCCTGCCCCGGATTT | |
| | CTGTGGGCCCTCGTGATCAGCACCTGTCTGGAATTCAGCATGGCCCAGACCGTGACTCAGTCCCAGC | |
| | CCGAGATGAGCGTGCAGGAAGCCGAGACAGTGACCCTGAGCTGCACCTACGACACCAGCGAGAGCG | |
| | ACTACTACCTGTTCTGGTACAAGCAGCCCCCCAGCCGGCAGATGATCCTCGTGATTAGACAGGAAGC | |
| | CTATAAGCAGCAGAACGCCACCGAGAACAGATTCAGCGTGAACTTCCAGAAGGCCGCCAAGTCCTT | |
| | CAGCCTGAAGATCAGCGACAGCCAGCTGGGCGACGCCGCCATGTACTTTTGTGCCTACCTGGGCACC | |
| | GGCACCTACAAGTACATCTTCGGCACAGGCACCCGGCTGAAGGTGCTGGCCAACATCCAGAACCCCG | |
| | ACCCTGCCGTGTACCAGCTGAGAGACAGCAAGAGCAGCGACAAGAGCGTGTGCCTGTTCACCGACTT | |
| | CGACAGCCAGACCAACGTGTCCCAGAGCAAGGACAGCGACGTGTACATCACCGATAAGACCGTGCT | |
| | GGACATGCGGAGCATGGACTTCAAGAGCAACAGCGCCGTGGCCTGGTCCAACAAGTCCGATTTCGCC | |
| | TGCGCCAACGCCTTCAACAACAGCATTATCCCCGAGGACACATTCTTCCCAAGCCCCGAGAGCAGCT | |
| | GCGACGTGAAGCTGGTGGAAAAGAGCTTCGAGACAGACACCAACCTGAACTTCCAGAACCTGTCCG | |
| | TGATCGGCTTCAGAATCCTGCTGCTGAAAGTGGCCGGCTTCAATCTGCTGATGACCCTGCGGCTGTG | |
| | GTCCAGCTG | |
| Valpha | MACPGFLWALVISTCLEFSMAQTVTQSQPEMSVQEAETVTLSCTYDTSESDYYLFWYKQPPSRQMILVIR | SEQ ID NO: 58 |
| (Peptide) | QEAYKQQNATENRFSVNFQKAAKSFSLKISDSQLGDAAMYFCAYLGTGTYKYIFGTGTRLKVLAN | |
| Valpha constant | IQNPDPAVYQLRDSKSSDKSVCLFTDFDSQTNVSQSKDSDVYITDKTVLDMRSMDFKSNSAVAWSNKSD | SEQ ID NO: 59 |
| (Peptide) | FACANAFNNSIIPEDTFFPSPESSCDVKLVEKSFETDTNLNFQNLSVIGFRILLLKVAGFNLLMTLRLWSS | |
| Valpha- | MACPGFLWALVISTCLEFSMAQTVTQSQPEMSVQEAETVTLSCTYDTSESDYYLFWYKQPPSRQMI | SEQ ID NO: 60 |
| Valpha constant | LVIRQEAYKQQNATENRFSVNFQKAAKSFSLKISDSQLGDAAMYFCAYLGTGTYKYIFGTGTRLKVL | |
| (Peptide) | ANIQNPDPAVYQLRDSKSSDKSVCLFTDFDSQTNVSQSKDSDVYITDKTVLDMRSMDFKSNSAVAWSNKSD | |
| | FACANAFNNSIIPEDTFFPSPESSCDVKLVEKSFETDTNLNFQNLSVIGFRILLLKVAGFNLLMTLRLWSS | |
| Vbeta | MGPQLLGYVVLCLLGAGPLEAQVTQNPRYLITVTGKKLTVTCSQNMNHEYMSWYRQDPGLGLRQIYYS | SEQ ID NO: 61 |
| (Peptide) | MNVEVTDKGDVPEGYKVSRKEKRNFPLILESPSPNQTSLYFCASSSGGLGYTFGSGTRLTVVE | |
| Vbeta constant | DLNKVFPPEVAVFEPSEAEISHTQKATLVCLATGFFPDHVELSWWVNGKEVHSGVSTDPQPLKEQPALND | SEQ ID NO: 62 |
| (Peptide) | SRYCLSSRLRVSATFWQNPRNHFRCQVQFYGLSENDEWTQDRAKPVTQIVSAEAWGRADCGFTSVSYQ | |
| | QGVLSATILYEILLGKATLYAVLVSALVLMAMVKRKDF | |
| Vbeta- | MGPQLLGYVVLCLLGAGPLEAQVTQNPRYLITVTGKKLTVTCSQNMNHEYMSWYRQDPGLGLRQ | SEQ ID NO: 63 |
| Vbeta constant | IYYSMNVEVTDKGDVPEGYKVSRKEKRNFPLILESPSPNQTSLYFCASSSGGLGYTFGSGTRLTVVED | |
| (Peptide) | LNKVFPPEVAVFEPSEAEISHTQKATLVCLATGFFPDHVELSWWVNGKEVHSGVSTDPQPLKEQPALNDS | |
| | RYCLSSRLRVSATFWQNPRNHFRCQVQFYGLSENDEWTQDRAKPVTQIVSAEAWGRADCGFTSVSYQQ | |
| | GVLSATILYEILLGKATLYAVLVSALVLMAMVKRKDF | |
| Vbeta-Vbeta | MGPQLLGYVVLCLLGAGPLEAQVTQNPRYLITVTGKKLTVTCSQNMNHEYMSWYRQDPGLGLRQ | SEQ ID NO: 64 |
| constant-P2A- | IYYSMNVEVTDKGDVPEGYKVSRKEKRNFPLILESPSPNQTSLYFCASSSGGLGYTFGSGTRLTVVED | |
| Valpha-Valpha | LNKVFPPEVAVFEPSEAEISHTQKATLVCLATGFFPDHVELSWWVNGKEVHSGVSTDPQPLKEQPALNDS | |
| constant | RYCLSSRLRVSATFWQNPRNHFRCQVQFYGLSENDEWTQDRAKPVTQIVSAEAWGRADCGFTSVSYQQ | |
| (Peptide) | GVLSATILYEILLGKATLYAVLVSALVLMAMVKRKDFGSGATNFSLLKQAGDVEENPGPMACPGFLWAL | |
| <Full Length | VISTCLEFSMAQTVTQSQPEMSVQEAETVTLSCTYDTSESDYYLFWYKQPPSRQMILVIRQEAYKQ | |
| TCR sequence> | QNATENRFSVNFQKAAKSFSLKISDSQLGDAAMYFCAYLGTGTYKYIFGTGTRLKVLANIQNPDPAV | |
| | YQLRDSKSSDKSVCLFTDFDSQTNVSQSKDSDVYITDKTVLDMRSMDFKSNSAVAWSNKSDFACANAFN | |
| | NSIIPEDTFFPSPESSCDVKLVEKSFETDTNLNFQNLSVIGFRILLLKVAGFNLLMTLRLWSS | |

TABLE 5
(MSLN₅₃₀₋₅₃₈, clone 5)

| | | |
|---|---|---|
| CDR3 TCRα | CAGGMESGGGADGLTF | SEQ ID NO: 10 |
| peptide | | |
| CDR3 TCRα | tgc gct ggc gga atg gaa tct ggc ggc gga gcc gat ggc ctg acc ttt | SEQ ID NO: 65 |
| DNA | | |
| CDR3 TCRβ | CASTSTGGLKNTEAFF | SEQ ID NO: 13 |
| peptide | | |
| CDR3 TCRβ | tgt gcc agc aca agc aca ggc ggc ctg aag aac acc gag gca ttc ttt | SEQ ID NO: 66 |
| DNA | | |
| CO-Vbeta-Vbeta | ATGGCGCGCCACCATGGGAACAAGCCTGCTGTGTTGGATGGCCCTGTGTCTGCTGGGAGCCGACCAT | SEQ ID NO: 67 |
| constant-P2A- | GCCGATACAGGCGTGTCCCAGAACCCCCGGCACAAGATCACCAAGCGGGGCCAGAACGTGACCTTC | |
| Valpha-Valpha | AGATGCGACCCCATCAGCGAGCACAACCGGCTGTACTGGTACAGACAGACCCTGGGCCAGGGCCCC | |
| constant | GAGTTCCTGACCTACTTCCAGAACGAGGCCCAGCTGGAAAAGAGCCGGCTGCTGAGCGACAGATTC | |
| (DNA) | AGCGCCGAAAGACCCAAGGGCAGCTTCAGCACCCTGGAAATCCAGCGGACCGAGCAGGGCGACAGC | |
| | GCCATGTATCTGTGTGCCAGCACAAGCACAGGCGGCCTGAAGAACACCGAGGCATTCTTTGGGCAGG | |
| | GCACCCGGCTGACCGTGGTGGAAGATCTGAACAAGGTGTTCCCCCCAGAGGTGGCCGTGTTCGAGCC | |
| | TTCTGAGGCCGAGATCAGCCACACCCAGAAAGCCACCCTCGTGTGCCTGGCCACCGGCTTTTTCCCC | |
| | GACCACGTGGAACTGTCTTGGTGGGTCAACGGCAAAGAGGTGCACAGCGGCGTGTCCACCGATCCCC | |
| | AGCCTCTGAAAGAACAGCCCGCCCTGAACGACAGCCGGTACTGCCTGAGCAGCAGACTGAGAGTGT | |
| | CCGCCACCTTCTGGCAGAACCCCAGAAACCACTTCAGGTGCCAGGTGCAGTTCTACGGCCTGAGCGA | |
| | GAACGACGAGTGGACCCAGGACAGAGCCAAGCCCGTGACCCAGATCGTGTCTGCCGAAGCCTGGGG | |
| | CAGAGCCGATTGCGGCTTTACCAGCGTGTCCTATCAGCAGGGCGTGCTGAGCGCCACAATCCTGTAC | |
| | GAGATCCTGCTGGGCAAGGCCACCCTGTACGCCGTGCTGGTGTCTGCCCTGGTGCTGATGGCCATGG | |
| | TCAAGCGGAAGGACTTCGGTTCCGGAGCCACGAACTTCTCTCTGTTAAAGCAAGCAGGAGACGTGGAAGA | |
| | AAACCCCGGTCCCGTCCCATGGTGCTGAAGTTCTCCGTGTCCATCCTGTGGATCCAGCTGGCCTGGGT | |
| | GTCCACCCAGCTGCTGGAACAGTCCCCTCAGTTCCTGAGCATCCAGGAAGGCGAGAACCTGACCGTG | |
| | TACTGCAACAGCAGCAGCGTGTTCAGCAGCCTGCAGTGGTACAGGCAGGAACCAGGCGAGGGACCA | |
| | GTGCTGCTCGTGACTGTCGTGACAGGCGGCGAAGTGAAGAAGCTGAAGCGGCTGACCTTCCAGTTCG | |
| | GCGACGCCAGAAAGGACAGCTCCCTGCACATTACAGCCGCCCAGACAGGCGACACCGGCCTGTACC | |
| | TGTGCGCTGGCGGAATGGAATCTGGCGGCGGAGCCGATGGCCTGACCTTTGGCAAGGGCACACACCT | |
| | GATCATCCAGCCCTACATCCAGAATCCCGACCCCGCCGTGTACCAGCTGAGAGACAGCAAGAGCAG | |
| | CGACAAGAGCGTGTGTCTGTTCACCGACTTCGACAGCCAGACCAATGTGTCCCAGTCCAAGGACAGC | |
| | GACGTGTACATCACCGACAAGACCGTGCTGGACATGCGGAGCATGGACTTCAAGAGCAACAGCGCC | |
| | GTGGCCTGGTCCAACAAGAGCGATTTCGCCTGCGCCAACGCCTTCAACAACAGCATTATCCCCGAGG | |
| | ACACATTCTTCCCAAGCCCCGAGAGCAGCTGCGACGTGAAGCTGGTGGAAAAGTCCTTCGAGACAG | |
| | ACACCAACCTGAATTTCCAGAATCTGAGCGTGATCGGCTTCCGCATCCTGCTGCTGAAGGTGGCCGG | |
| | CTTCAACCTGCTGATGACCCTGAGACTGTGGTCCTCCTGAGTCGAC | |
| Valpha | MVLKFSVSILWIQLAWVSTQLLEQSPQFLSIQEGENLTVYCNSSSVFSSLQWYRQEPGEGPVLLVTVVTGG | SEQ ID NO: 68 |
| (Peptide) | EVKKLKRLTFQFGDARKDSSLHITAAQTGDTGLYLCAGGMESGGGADGLTFGKGTHLIIQPY | |
| Valpha constant | IQNPDPAVYQLRDSKSSDKSVCLFTDFDSQTNVSQSKDSDVYITDKTVLDMRSMDFKSNSAVAWSNKSD | SEQ ID NO: 69 |
| (Peptide) | FACANAFNNSIIPEDTFFPSPESSCDVKLVEKSFETDTNLNFQNLSVIGFRILLLKVAGFNLLMTLRLWSS | |
| Valpha- | MVLKFSVSILWIQLAWVSTQLLEQSPQFLSIQEGENLTVYCNSSSVFSSLQWYRQEPGEGPVLLVTV | SEQ ID NO: 70 |
| Valpha constant | VTGGEVKKLKRLTFQFGDARKDSSLHITAAQTGDTGLYLCAGGMESGGGADGLTFGKGTHLIIQPYI | |
| (Peptide) | QNPDPAVYQLRDSKSSDKSVCLFTDFDSQTNVSQSKDSDVYITDKTVLDMRSMDFKSNSAVAWSNKSDF | |
| | ACANAFNNSIIPEDTFFPSPESSCDVKLVEKSFETDTNLNFQNLSVIGFRILLLKVAGFNLLMTLRLWSS | |
| Vbeta | MGTSLLCWMALCLLGADHADTGVSQNPRHKITKRGQNVTFRCDPISEHNRLYWYRQTLGQGPEFLTYF | SEQ ID NO: 71 |
| (Peptide) | QNEAQLEKSRLLSDRFSAERPKGSFSTLEIQRTEQGDSAMYLCASTSTGGLKNTEAFFGQGTRLTVVE | |
| Vbeta constant | DLNKVFPPEVAVFEPSEAEISHTQKATLVCLATGFFPDHVELSWWVNGKEVHSGVSTDPQPLKEQPALND | SEQ ID NO: 72 |
| (Peptide) | SRYCLSSRLRVSATFWQNPRNHFRCQVQFYGLSENDEWTQDRAKPVTQIVSAEAWGRADCGFTSVSYQ | |
| | QGVLSATILYEILLGKATLYAVLVSALVLMAMVKRKDF | |
| Vbeta- | MGTSLLCWMALCLLGADHADTGVSQNPRHKITKRGQNVTFRCDPISEHNRLYWYRQTLGQGPEF | SEQ ID NO: 73 |
| Vbeta constant | LTYFQNEAQLEKSRLLSDRFSAERPKGSFSTLEIQRTEQGDSAMYLCASTSTGGLKNTEAFFGQGTRL | |
| (Peptide) | TVVEDLNKVFPPEVAVFEPSEAEISHTQKATLVCLATGFFPDHVELSWWVNGKEVHSGVSTDPQPLKEQP | |
| | ALNDSRYCLSSRLRVSATFWQNPRNHFRCQVQFYGLSENDEWTQDRAKPVTQIVSAEAWGRADCGFTS | |
| | VSYQQGVLSATILYEILLGKATLYAVLVSALVLMAMVKRKDF | |
| Vbeta-Vbeta | MGTSLLCWMALCLLGADHADTGVSQNPRHKITKRGQNVTFRCDPISEHNRLYWYRQTLGQGPEF | SEQ ID NO: 74 |
| constant-P2A- | LTYFQNEAQLEKSRLLSDRFSAERPKGSFSTLEIQRTEQGDSAMYLCASTSTGGLKNTEAFFGQGTRL | |
| Valpha-Valpha | TVVEDLNKVFPPEVAVFEPSEAEISHTQKATLVCLATGFFPDHVELSWWVNGKEVHSGVSTDPQPLKEQP | |
| constant | ALNDSRYCLSSRLRVSATFWQNPRNHFRCQVQFYGLSENDEWTQDRAKPVTQIVSAEAWGRADCGFTS | |
| (Peptide) | VSYQQGVLSATILYEILLGKATLYAVLVSALVLMAMVKRKDFGSGATNFSLLKQAGDVEENPGPMVLKF | |
| <Full Length | SVSILWIQLAWVSTQLLEQSPQFLSIQEGENLTVYCNSSSVFSSLQWYRQEPGEGPVLLVTVVTGGE | |
| TCR sequence> | VKKLKRLTFQFGDARKDSSLHITAAQTGDTGLYLCAGGMESGGGADGLTFGKGTHLIIQPYIQNPDP | |
| | AVYQLRDSKSSDKSVCLFTDFDSQTNVSQSKDSDVYITDKTVLDMRSMDFKSNSAVAWSNKSDFACAN | |
| | AFNNSIIPEDTFFPSPESSCDVKLVEKSFETDTNLNFQNLSVIGFRILLLKVAGFNLLMTLRLWSS | |

TABLE 6
(MSLN₅₃₀₋₅₃₈, clone 6)

| | | |
|---|---|---|
| CDR3 TCRα | CALDTGFQKLVF | SEQ ID NO: 11 |
| peptide | | |
| CDR3 TCRα | tgc gcc ctg gat acc ggc ttt cag aaa ctg gtg ttc | SEQ ID NO: 75 |
| DNA | | |
| CDR3 TCRβ | CASSSLGDRNTEAFF | SEQ ID NO: 14 |
| peptide | | |
| CDR3 TCRβ | tgt gcc agc agc agc ctg ggc gac cgg aac acc gag gca ttc ttt | SEQ ID NO: 76 |
| DNA | | |
| CO-Vbeta-Vbeta | ATGGCGCGCCACCATGGGACCTCAGCTGCTGGGATACGTGGTGCTGTGTCTGCTGGGAGCCGGACCT | SEQ ID NO: 77 |
| constant-P2A- | CTGGAAGCCCAAGTGACCCAGAACCCCAGATACCTGATCACCGTGACCGGCAAGAAACTGACCGTG | |
| Valpha-Valpha | ACCTGCAGCCAGAACATGAACCACGAGTACATGAGCTGGTACAGACAGGACCCCGGCCTGGGCCTG | |
| constant | CGGCAGATCTACTACAGCATGAACGTGGAAGTGACCGACAAGGGCGACGTGCCCGAGGGCTACAAG | |
| (DNA) | GTGTCCCGGAAAGAGAAGCGGAACTTCCCACTGATCCTGGAAAGCCCCAGCCCCAACCAGACCAGC | |
| | CTGTACTTCTGTGCCAGCAGCAGCCTGGGCGACCGGAACACCGAGGCATTCTTTGGGCAGGGCACCC | |
| | GGCTGACCGTGGTGGAAGATCTGAACAAGGTGTTCCCCCCAGAGGTGGCCGTGTTCGAGCCTTCTGA | |
| | GGCCGAGATCAGCCACACCCAGAAAGCCACCCTCGTGTGCCTGGCCACCGGCTTTTTCCCCGACCAC | |
| | GTGGAACTGTCTTGGTGGGTCAACGGCAAAGAGGTGCACAGCGGCGTGTCCACCGATCCCCAGCCTC | |
| | TGAAAGAACAGCCCGCCCTGAACGACAGCCGGTACTGCCTGAGCAGCAGACTGAGAGTGTCCGCCA | |
| | CCTTCTGGCAGAACCCCCGGAACCACTTCAGATGCCAGGTGCAGTTCTACGGCCTGAGCGAGAACGA | |
| | CGAGTGGACCCAGGACAGAGCCAAGCCCGTGACACAGATCGTGTCTGCCGAAGCCTGGGGCAGAGC | |
| | CGATTGCGGCTTTACCAGCGTGTCCTATCAGCAGGGCGTGCTGAGCGCCACAATCCTGTACGAGATC | |
| | CTGCTGGGCAAGGCCACCCTGTACGCCGTGCTGGTGTCAGCCCTGGTGCTGATGGCCATGGTCAAGC | |
| | GGAAGGACTTCGGTTCCGGAGCCACGAACTTCTCTCTGTTAAAGCAAGCAGGAGACGTGGAAGAAAACCC | |
| | CGGTCCCGTCCCATGAACTACAGCCCTGGCCTGGTGTCCCTGATTCTCCTGCTGCTGGGGCGGACCAG | |
| | AGGCAACTCCGTGACTCAGATGGAAGGCCCCGTGACCCTGAGCGAAGAGGCCTTCCTGACCATCAAT | |
| | TGCACCTACACCGCCACAGGCTACCCCAGCCTGTTTTGGTACGTGCAGTACCCCGGCGAGGGACTGC | |
| | AGCTGCTGCTGAAGGCCACCAAGGCCGACGATAAGGGCAGCAACAAGGGCTTCGAGGCCACCTACA | |
| | GAAAAGAGACAACCAGCTTCCACCTGGAAAAGGGCAGCGTGCAGGTGTCCGACAGCGCCGTGTATT | |
| | TCTGCGCCCTGGATACCGGCTTTCAGAAACTGGTGTTCGGCACCGGCACCAGACTGCTGGTGTCCCC | |
| | CAACATCCAGAACCCCGACCCTGCCGTGTATCAGCTGCGGGACAGCAAGAGCAGCGACAAGAGCGT | |
| | GTGTCTGTTCACCGACTTCGACAGCCAGACCAACGTGTCCCAGAGCAAGGACTCCGACGTGTACATC | |
| | ACCGACAAGACCGTGCTGGACATGCGGAGCATGGACTTCAAGAGCAACTCCGCCGTGGCCTGGTCC | |
| | AACAAGAGCGATTTCGCCTGCGCCAACGCCTTCAACAACAGCATTATCCCCGAGGACACATTCTTCC | |
| | CAAGCCCCGAGAGCAGCTGCGACGTGAAGCTGGTGGAAAAGAGCTTCGAGACAGACACCAACCTGA | |
| | ACTTCCAGAACCTGAGCGTGATCGGCTTCCGGATCCTGCTGCTGAAAGTGGCCGGCTTCAACCTGCT | |
| | GATGACCCTGCGGCTGTGGTCCAGCTGAGTCGAC | |
| Valpha | MNYSPGLVSLILLLLGRTRGNSVTQMEGPVTLSEEAFLTINCTYTATGYPSLFWYVQYPGEGLQLLLKAT | SEQ ID NO: 78 |
| (Peptide) | KADDKGSNKGFEATYRKETTSFHLEKGSVQVSDSAVYFCALDTGFQKLVFGTGTRLLVSPN | |
| Valpha constant | IQNPDPAVYQLRDSKSSDKSVCLFTDFDSQTNVSQSKDSDVYITDKTVLDMRSMDFKSNSAVAWSNKSD | SEQ ID NO: 79 |
| (Peptide) | FACANAFNNSIIPEDTFFPSPESSCDVKLVEKSFETDTNLNFQNLSVIGFRILLLKVAGFNLLMTLRLWSS | |
| Valpha- | MNYSPGLVSLILLLLGRTRGNSVTQMEGPVTLSEEAFLTINCTYTATGYPSLFWYVQYPGEGLQLL | SEQ ID NO: 80 |
| Valpha constant | LKATKADDKGSNKGFEATYRKETTSFHLEKGSVQVSDSAVYFCALDTGFQKLVFGTGTRLLVSPNIQ | |
| (Peptide) | NPDPAVYQLRDSKSSDKSVCLFTDFDSQTNVSQSKDSDVYITDKTVLDMRSMDFKSNSAVAWSNKSDFA | |
| | CANAFNNSIIPEDTFFPSPESSCDVKLVEKSFETDTNLNFQNLSVIGFRILLLKVAGFNLLMTLRLWSS | |
| Vbeta | MGPQLLGYVVLCLLGAGPLEAQVTQNPRYLITVTGKKLTVTCSQNMNHEYMSWYRQDPGLGLRQIYYS | SEQ ID NO: 81 |
| (Peptide) | MNVEVTDKGDVPEGYKVSRKEKRNFPLILESPSPNQTSLYFCASSSLGDRNTEAFFGQGTRLTVVE | |
| Vbeta constant | DLNKVFPPEVAVFEPSEAEISHTQKATLVCLATGFFPDHVELSWWVNGKEVHSGVSTDPQPLKEQPALND | SEQ ID NO: 82 |
| (Peptide) | SRYCLSSRLRVSATFWQNPRNHFRCQVQFYGLSENDEWTQDRAKPVTQIVSAEAWGRADCGFTSVSYQ | |
| | QGVLSATILYEILLGKATLYAVLVSALVLMAMVKRKDF | |
| Vbeta- | MGPQLLGYVVLCLLGAGPLEAQVTQNPRYLITVTGKKLTVTCSQNMNHEYMSWYRQDPGLGLRQ | SEQ ID NO: 83 |
| Vbeta constant | IYYSMNVEVTDKGDVPEGYKVSRKEKRNFPLILESPSPNQTSLYFCASSSLGDRNTEAFFGQGTRLTV | |
| (Peptide) | VEDLNKVFPPEVAVFEPSEAEISHTQKATLVCLATGFFPDHVELSWWVNGKEVHSGVSTDPQPLKEQPAL | |
| | NDSRYCLSSRLRVSATFWQNPRNHFRCQVQFYGLSENDEWTQDRAKPVTQIVSAEAWGRADCGFTSVS | |
| | YQQGVLSATILYEILLGKATLYAVLVSALVLMAMVKRKDF | |
| Vbeta-Vbeta | MGPQLLGYVVLCLLGAGPLEAQVTQNPRYLITVTGKKLTVTCSQNMNHEYMSWYRQDPGLGLRQ | SEQ ID NO: 84 |
| constant-P2A- | IYYSMNVEVTDKGDVPEGYKVSRKEKRNFPLILESPSPNQTSLYFCASSSLGDRNTEAFFGQGTRLTV | |
| Valpha-Valpha | VEDLNKVFPPEVAVFEPSEAEISHTQKATLVCLATGFFPDHVELSWWVNGKEVHSGVSTDPQPLKEQPAL | |
| constant | NDSRYCLSSRLRVSATFWQNPRNHFRCQVQFYGLSENDEWTQDRAKPVTQIVSAEAWGRADCGFTSVS | |
| (Peptide) | YQQGVLSATILYEILLGKATLYAVLVSALVLMAMVKRKDFGSGATNFSLLKQAGDVEENPGPMNYSPGL | |
| <Full Length | VSLILLLLGRTRGNSVTQMEGPVTLSEEAFLTINCTYTATGYPSLFWYVQYPGEGLQLLLKATKAD | |
| TCR sequence> | DKGSNKGFEATYRKETTSFHLEKGSVQVSDSAVYFCALDTGFQKLVFGTGTRLLVSPNIQNPDPAVY | |
| | QLRDSKSSDKSVCLFTDFDSQTNVSQSKDSDVYITDKTVLDMRSMDFKSNSAVAWSNKSDFACANAFNN | |
| | SIIPEDTFFPSPESSCDVKLVEKSFETDTNLNFQNLSVIGFRILLLKVAGFNLLMTLRLWSS | |

What is claimed is:

1. A mesothelin-specific binding protein comprising:
a T cell receptor (TCR) α-chain variable (V_{α}) domain comprising
a TCRα CDR3 having a peptide sequence of CAASGNTDKLIF (SEQ ID NO:3); a TCRα CDR3 having a peptide sequence of CAFYMDSNYQLIW (SEQ ID NO:4); or a TCRα CDR3 having a peptide sequence of CAVIPNNNARLMF (SEQ ID NO:5); and
a TCR β-chain variable (V_{β}) domain.

2. A mesothelin-specific binding protein comprising:
a T cell receptor (TCR) β-chain variable (V_{β}) domain comprising
a TCRβ CDR3 having a peptide sequence of CASRPGWSYEQYF (SEQ ID NO:6); a TCRβ CDR3 having a peptide sequence of CASSEWTAEQYF (SEQ ID NO:7); or a TCRβ CDR3 having a peptide sequence of CASGQGTEAFF (SEQ ID NO:8); and
a TCR α-chain variable (V_{α}) domain.

3. The mesothelin-specific binding protein of claim 1 or 2, comprising:
a TCRα CDR3 having a peptide sequence of CAASGNTDKLIF (SEQ ID NO:3) and a TCRβ CDR3 having a peptide sequence of CASRPGWSYEQYF (SEQ ID NO:6);
a TCRα CDR3 having a peptide sequence of CAFYMDSNYQLIW (SEQ ID NO:4) and a TCRβ CDR3 having a peptide sequence of CASSEWTAEQYF (SEQ ID NO:7); or
a TCRα CDR3 having a peptide sequence of CAVIPNNNARLMF (SEQ ID NO:5) and a TCRβ CDR3 having a peptide sequence of CASGQGTEAFF (SEQ ID NO:8).

4. The mesothelin-specific binding protein of any one of claims 1 to 3, wherein the mesothelin-specific binding protein binds to a tetramer comprising amino acids 20-28 of human mesothelin with a K_{d} of less than 10⁻⁸ M, less than 10⁻⁹ M, less than 10⁻¹⁰ M, less than 10⁻¹¹ M, less than 10⁻¹² M, or less than 10⁻¹³ M.

5. The mesothelin-specific binding protein of claim 3, wherein the mesothelin-specific binding protein comprises a Vα domain comprising the peptide sequence of SEQ ID NO: 23 and a V_{β} domain comprising the peptide sequence of SEQ ID NO: 26.

6. The mesothelin-specific binding protein of claim 3, wherein the mesothelin-specific binding protein comprises a V_{α} domain comprising the peptide sequence of SEQ ID NO: 25 and a V_{β} domain comprising the peptide sequence of SEQ ID NO: 28.

7. The mesothelin-specific binding protein of claim 3, wherein the mesothelin-specific binding protein comprises the peptide sequence of SEQ ID NO: 29.

8. The mesothelin-specific binding protein of claim 3, wherein the mesothelin-specific binding protein comprises a V_{α} domain comprising the peptide sequence of SEQ ID NO: 33 and a V_{β} domain comprising the peptide sequence of SEQ ID NO: 36.

9. The mesothelin-specific binding protein of claim 3, wherein the mesothelin-specific binding protein comprises a V_{α} domain comprising the peptide sequence of SEQ ID NO: 35 and a V_{β} domain comprising the peptide sequence of SEQ ID NO: 38.

10. The mesothelin-specific binding protein of claim 3, wherein the mesothelin-specific binding protein comprises peptide sequence of SEQ ID NO: 39.

11. The mesothelin-specific binding protein of claim 3, wherein the mesothelin-specific binding protein comprises a V_{α} domain comprising the peptide sequence of SEQ ID NO: 43 and a V_{β} domain comprising the peptide sequence of SEQ ID NO: 46.

12. The mesothelin-specific binding protein of claim 3, wherein the mesothelin-specific binding protein comprises a V_{α} domain comprising the peptide sequence of SEQ ID NO: 45 and a V_{β} domain comprising the peptide sequence of SEQ ID NO: 48.

13. The mesothelin-specific binding protein of claim 3, wherein the mesothelin-specific binding protein comprises the peptide sequence of SEQ ID NO: 49.

14. A mesothelin-specific binding protein comprising:
a T cell receptor (TCR) α-chain variable (V_{α}) domain comprising
a TCRα CDR3 having a peptide sequence of CAYLGTGTYKYIF (SEQ ID NO:9); a TCRα CDR3 having a peptide sequence of CAGGMESGGGADGLTF (SEQ ID NO:10); or a TCRα CDR3 having a peptide sequence of CALDTGFQKLVF (SEQ ID NO:11); and
a TCR β-chain variable (V_{β}) domain.

15. A mesothelin-specific binding protein comprising:
a T cell receptor (TCR) β-chain variable (V_{β}) domain comprising
a TCRβ CDR3 having a peptide sequence of CASSSGGLGYTF (SEQ ID NO:12); a TCRβ CDR3 having a peptide sequence of CASTSTGGLKNTEAFF (SEQ ID NO:13); or a TCRβ CDR3 having a peptide sequence of CASSSLGDRNTEAFF (SEQ ID NO:14); and
a TCR α-chain variable (V_{α}) domain.

16. The mesothelin-specific binding protein of claim 14 or 15, comprising:
a TCRα CDR3 having a peptide sequence of CAYLGTGTYKYIF (SEQ ID NO:9) and a TCRβ CDR3 having a peptide sequence of CASSSGGLGYTF (SEQ ID NO:12);
a TCRα CDR3 having a peptide sequence of CAGGMESGGGADGLTF (SEQ ID NO:10) and a TCRβ CDR3 having a peptide sequence of CASTSTGGLKNTEAFF (SEQ ID NO:13); or
a TCRα CDR3 having a peptide sequence of CALDTGFQKLVF (SEQ ID NO:11) and a TCRβ CDR3 having a peptide sequence of CASSSLGDRNTEAFF (SEQ ID NO:14).

17. The mesothelin-specific binding protein of any one of claims 14 to 16, wherein the mesothelin-specific binding protein binds to a tetramer comprising amino acids 530-538 of human mesothelin with a K_{d} of less than 10⁻⁸ M, less than 10⁻⁹ M, less than 10⁻¹⁰ M, less than 10⁻¹¹ M, less than 10⁻¹² M, or less than 10⁻¹³ M.

18. The mesothelin-specific binding protein of claim 17, wherein the mesothelin-specific binding protein comprises a V_{α} domain comprising the peptide sequence of SEQ ID NO: 58 and a V_{β} domain comprising the peptide sequence of SEQ ID NO: 61.

19. The mesothelin-specific binding protein of claim 17, wherein the mesothelin-specific binding protein comprises a V_{α} domain comprising the peptide sequence of SEQ ID NO: 60 and a V_{β} domain comprising the peptide sequence of SEQ ID NO: 63.

20. The mesothelin-specific binding protein of claim 17, wherein the mesothelin-specific binding protein comprises the peptide sequence of SEQ ID NO: 64.

21. The mesothelin-specific binding protein of claim 17, wherein the mesothelin-specific binding protein comprises a V_{α} domain comprising the peptide sequence of SEQ ID NO: 68 and a V_{β} domain comprising the peptide sequence of SEQ ID NO: 71.

22. The mesothelin-specific binding protein of claim 17, wherein the mesothelin-specific binding protein comprises a V_{α} domain comprising the peptide sequence of SEQ ID NO: 70 and a V_{β} domain comprising the peptide sequence of SEQ ID NO: 73.

23. The mesothelin-specific binding protein of claim 17, wherein the mesothelin-specific binding protein comprises the peptide sequence of SEQ ID NO: 74.

24. The mesothelin-specific binding protein of claim 17, wherein the mesothelin-specific binding protein comprises a V_{α} domain comprising the peptide sequence of SEQ ID NO: 78 and a V_{β} domain comprising the peptide sequence of SEQ ID NO: 81.

25. The mesothelin-specific binding protein of claim 157 wherein the mesothelin-specific binding protein comprises a V_{α} domain comprising the peptide sequence of SEQ ID NO: 80 and a V_{β} domain comprising the peptide sequence of SEQ ID NO: 83.

26. The mesothelin-specific binding protein of claim 17, wherein the mesothelin-specific binding protein comprises the peptide sequence of SEQ ID NO: 84

27. A cell that overexpresses a mesothelin-specific binding protein of any one of claims 1 to 26.

28. The cell of claim 27, wherein the cell overexpresses:
a molecule that interferes with inhibitory receptor expression;
a molecule that interferes with suppressive cytokine signaling;
a molecule that renders T cells resistant to a program of T cell exhaustion and/or promotes resident memory;
a chimeric costimulatory receptor; and/or
an anti-tumor factor.

29. The cell of claim 27 or claim 28, wherein the cell is modified to reduce expression of a transforming growth factor beta (TGFβ) receptor.

30. The cell of claim 29, wherein the TGFβ receptor is TGFβR2 or TGFβR1 .

31. A method comprising delivering the mesothelin-specific binding protein of any one of claims 1 to 26 to a target cell to produce a cell that overexpresses the mesothelin-specific binding protein.

32. A method comprising delivering a construct that encodes a mesothelin-specific binding protein of any one of claims 1 to 26 to a target cell to produce a cell that overexpresses the mesothelin-specific binding protein.

33. The method of claim 31 or claim 32, wherein the delivery of the mesothelin-specific binding protein or construct comprises in vivo delivery to the target cell.

34. The method of claim 31 or claim 32, wherein the delivery of the mesothelin-specific binding protein or construct comprises ex vivo delivery to the target cell.

35. The method of any one of claims 31 to 34, wherein the target cell is a T cell, an NK cell, an NKT cell, a pluripotent cell, or a lymphocyte derived from a pluripotent cell.

36. The method of any one of claims 31 to 34, wherein the target cell is in a pool of target cells comprising a combination T cells, NK cells, NKT cells, pluripotent cells, or lymphocytes derived from pluripotent cells.

37. The method of any one of claims 31 to 34, wherein the target cell is a T cell and wherein the mesothelin-specific binding protein comprises a TCR.

38. The method of any one of claims 31 to 37, wherein the method further comprises delivering the cell that overexpresses the mesothelin-specific binding protein to a subject that has or is suspected of having a mesothelin-positive malignancy.

39. A method of treating a mesothelin-positive malignancy in a subject in need thereof, the method comprising administering to the subject a composition comprising cells of any one of claims 27 to 30.

40. The method of claim 39, wherein the method further comprises administering a mesothelin peptide or a construct encoding a mesothelin peptide to the subject.

41. The method of claim 40, wherein the mesothelin peptide comprises the amino acid sequence of SEQ ID NO: 1 or SEQ ID NO: 2.

42. The method of any one of claims 39 to 41, wherein the method further comprises administering a CD40 agonist to the subject.

43. The method of any one of claims 39 to 42, wherein the method further comprises administering an adjuvant to the subject.

44. The method of claim 43, wherein the adjuvant is polyinosinic:polycytidylic acid (Poly:IC).

45. The method of any one of claims 39 to 442, wherein the method further comprises administering a cytokine to the subject.

46. The method of claim 45, wherein the cytokine is IL-2.

47. The method of any one of claims 39 to 46, wherein the method comprises (a) administering the composition comprising the cells of any one of claims 27 to 30 and (b) administering at a later timepoint (i) a mesothelin peptide or a construct encoding a mesothelin peptide and (ii) an adjuvant to the subject.
